(19) United States
(12) Patent Application Publication
(10) Pub. No.: US 2020/0247730 A1
(43) Pub. Date: Aug. 6, 2020

(54) Oxidative Conversion of Methane to C2 Hydrocarbons on Oxide Catalyst with Feed Comprising Organic Chloride

(71) Applicant: Sabic Global Technologies, B.V., Bergen op Zoom (NL)

(72) Inventors: Aghaddin MAMEDOV, Sugar Land, TX (US); Pankaj GAUTAM, Sugar Land, TX (US); Hoang NGUYEN, Sugar Land, TX (US)

(21) Appl. No.: 16/773,109

(22) Filed: Jan. 27, 2020

Related U.S. Application Data
(60) Provisional application No. 62/801,871, filed on Feb. 6, 2019.
(60) Provisional application No. 62/801,877, filed on Feb. 6, 2019.
(60) Provisional application No. 62/812,378, filed on Mar. 1, 2019.

(51) Int. Cl. C07C 2/84; C07C 11/04

(52) U.S. Cl. CPC C07C 2/84; C07C 11/04; C07C 2523/30; C07C 2521/08; C07C 2521/10; C07C 2523/10

(57) ABSTRACT

A process for producing C₂₊ hydrocarbons comprising introducing a reactant mixture to an oxidative coupling of methane (OCM) reactor comprising an OCM catalyst composition; wherein the reactant mixture comprises CH₄, O₂, and a chlorine intermediate precursor; wherein the chlorine intermediate precursor is present in the reactant mixture from about 1 ppm to about 100 ppm, based on the total volume of the reactant mixture; allowing the reactant mixture to contact the OCM catalyst composition and react via an OCM reaction to form a product mixture comprising unreacted methane and C₂₊ hydrocarbons; wherein the process for producing C₂₊ hydrocarbons is characterized by improved performance in the presence of the chlorine intermediate precursor; recovering at least a portion of the product mixture from the OCM reactor; and recovering the C₂₊ hydrocarbons from the product mixture. The OCM reactor is operated under autothermal or isothermal conditions.

### CROSS REFERENCE TO RELATED APPLICATIONS

This application claims priority to U.S. Provisional Application Nos. 62/801,871 filed on Feb. 6, 2019 and entitled “Oxidative Conversion of Methane to C2 Hydrocarbons on Mixed Oxide Catalyst with Feed Comprising Organic Chloride,” 62/801,877 filed on Feb. 6, 2019 and entitled “Oxidative Conversion of Methane on Lanthanum-Cerium Catalyst with Feed Comprising Organic Chloride,” and 62/812,378 filed on Mar. 1, 2019 and entitled “Oxidative Conversion of Methane to C2 Hydrocarbons on Mixed Oxide Catalysts in the Presence of ppm Amount of Chloride in the Feed,” the disclosure of which are hereby incorporated by reference in their entirety.

### TECHNICAL FIELD

The present disclosure relates to methods of producing hydrocarbons, more specifically methods of producing C₂₊ hydrocarbons, such as C₂₊ olefins (e.g., ethylene), via oxidative coupling of methane (OCM) with catalyst compositions based on oxides in the presence of chlorides in the OCM feed (e.g., using a feed comprising methane and at least one organic chloride).

### BACKGROUND

Hydrocarbons, and specifically olefins such as ethylene, are typically building blocks used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Oxidative coupling of the methane (OCM) has been the target of intense scientific and commercial interest for more than thirty years due to the tremendous potential of such technology to reduce costs, energy, and environmental emissions in the production of ethylene (C₂H₄). As an overall reaction, in the OCM, methane (CH₄) and oxygen (O₂) react exothermically over a catalyst to form C₂H₄, water (H₂O) and heat.

Ethylene can be produced by OCM as represented by Equations (I) and (II):

| | |
|---|---|
| 2CH₄ + O₂ → C₂H₄ + 2H₂O | ΔH = −67 kcal/mol (I) |
| 2CH₄ + 1/2O₂ → C₂H₆ + H₂O | ΔH = −42 kcal/mol (II) |

Oxidative conversion of methane to ethylene is exothermic. Excess heat produced from these reactions (Equations (I) and (II)) can push conversion of methane to deep oxidation products, carbon monoxide (CO) and carbon dioxide (CO₂), via Equations (III) and (IV), respectively, rather than the desired C₂ hydrocarbon product (e.g., ethylene):

| | |
|---|---|
| CH₄ + 1.5O₂ → CO + 2H₂O | ΔH = −124 kcal/mol (III) |
| CH₄ + 2O₂ → CO₂ + 2H₂O | ΔH = −192 kcal/mol (IV) |

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C—H bond breakage. The endothermic nature of the bond breakage is due to the chemical stability of methane, which is a chemically stable molecule due to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Further, the produced ethylene is highly reactive and can form unwanted and thermodynamically favored deep oxidation products.

Generally, in the OCM, CH₄ is first oxidatively converted into ethane (C₂H₆), and then into C₂H₄. CH₄ is activated heterogeneously on a catalyst surface, forming methyl radicals (e.g., CH₃.), which then couple in a gas phase to form C₂H₆. C₂H₆ subsequently undergoes dehydrogenation to form C₂H₄. An overall yield of desired C₂ hydrocarbons is reduced by non-selective reactions of methyl radicals with oxygen on the catalyst surface and/or in the gas phase, which produce the (undesirable) deep oxidation products carbon monoxide and carbon dioxide. Some of the best reported OCM outcomes encompass an approximately 20% conversion of methane and approximately 80% selectivity to desired C₂ hydrocarbons.

There are many catalyst systems developed for OCM processes, but such catalyst systems have many shortcomings. For example, conventional catalysts systems for OCM display catalyst performance problems, stemming from a need for high reaction temperatures to achieve desired conversions and selectivities, while displaying unstable performance across wide temperature ranges. Thus, there is an ongoing need for the development of improved OCM processes, for example processes that can allow for an increased performance in OCM reactions (e.g., increased selectivity to C₂ hydrocarbons, increased catalyst activity, increased methane conversion, increased catalyst stability, increased catalyst operational life time, etc.).

### BRIEF DESCRIPTION OF THE DRAWINGS

For a detailed description of the preferred aspects of the disclosed methods, reference will now be made to the accompanying drawing in which:

FIG. 1 displays a graph of oxygen (O₂) conversion as a function of time on stream in an oxidative coupling of the methane (OCM) reaction in the presence of chloride in the OCM feed;

FIG. 2 displays a graph of O₂ conversion as a function of time on stream in an OCM reaction in the absence of chloride in the OCM feed;

FIG. 3 displays another graph of O₂ conversion as a function of time on stream in an OCM reaction in the absence of chloride in the OCM feed;

FIG. 4 displays another graph of O₂ conversion as a function of time on stream in an OCM reaction in the presence of chloride in the OCM feed;

FIG. 5 displays a graph of O₂ conversion peak area as a function of time on stream in an OCM reaction in the presence of chloride in the OCM feed;

FIG. 6 displays a graph of CH₄ conversion as a function of temperature in an OCM reaction in the absence and in the presence of chloride in the OCM feed;

FIG. 7 displays a graph of C₂₊ selectivity as a function of temperature in an OCM reaction in the absence and in the presence of chloride in the OCM feed;

FIG. 8 displays a graph of O₂ conversion, CH₄ conversion, and C₂₊ selectivity as a function of time on stream in an OCM reaction in the presence of chloride in the OCM feed;

FIG. 9 displays a graph of a molar ratio of ethylene (C₂H₄) to ethane (C₂H₆) as a function of temperature in an OCM reaction in the absence and in the presence of chloride in the OCM feed;

FIG. 10 displays a graph of a molar ratio of carbon dioxide (CO₂) to carbon monoxide (CO) as a function of temperature in an OCM reaction in the absence and in the presence of chloride in the OCM feed;

FIG. 11 displays another graph of C₂₊ selectivity as a function of temperature in an OCM reaction in the absence and in the presence of chloride in the OCM feed;

FIG. 12 displays another graph of CH₄ conversion as a function of temperature in an OCM reaction in the absence and in the presence of chloride in the OCM feed;

FIG. 13 displays a graph of O₂ conversion as a function of temperature in an OCM reaction in the absence and in the presence of chloride in the OCM feed;

FIG. 14 displays yet another graph of C₂₊ selectivity as a function of temperature in an OCM reaction in the absence and in the presence of chloride in the OCM feed;

FIG. 15 displays another graph of a molar ratio of CO₂ to CO as a function of temperature in an OCM reaction in the absence and in the presence of chloride in the OCM feed;

FIG. 16 displays a graph of O₂ conversion, CH₄ conversion, and C₂₊ selectivity as a function of time on stream in an OCM reaction in the presence and in the absence of chloride in the OCM feed;

FIG. 17 displays a graph of C₂₊ selectivity and O₂ conversion as a function of temperature in an OCM reaction in the presence of chloride in the OCM feed, for OCM catalysts that have been pre-treated with chloride at room temperature and for OCM catalysts that have not been pre-treated;

FIG. 18 displays a graph of C₂₊ selectivity as a function of time on stream in an OCM reaction for various levels of chloride in the OCM feed;

FIG. 19 displays another graph of C₂₊ selectivity as a function of time on stream in an OCM reaction for various levels of chloride in the OCM feed, for OCM catalysts that have been pre-treated with chloride at room temperature and for OCM catalysts that have not been pre-treated;

FIG. 20 displays a graph showing the temperature profile of an autothermal reactor in the absence of chloride in the OCM feed;

FIG. 21 displays a graph showing the temperature profile of an autothermal reactor in the presence of chloride in the OCM feed;

FIG. 22 displays another graph showing the temperature profile of an autothermal reactor in the presence of chloride in the OCM feed;

FIG. 23 displays yet another graph showing the temperature profile of an autothermal reactor in the presence of chloride in the OCM feed; and

FIG. 24 displays another graph of C₂₊ selectivity and O₂ conversion as a function of temperature in an OCM reaction in the presence of chloride in the OCM feed, for OCM catalysts that have been pre-treated with chloride at room temperature and for OCM catalysts that have not been pre-treated.

### DETAILED DESCRIPTION

Disclosed herein is a process for producing C₂₊ hydrocarbons (e.g., C₂₊ olefins, C₂₊ alkanes, etc.) comprising (a) introducing a reactant mixture to an oxidative coupling of methane (OCM) reactor comprising an OCM catalyst composition; wherein the reactant mixture comprises methane (CH₄), oxygen (O₂), and a chlorine intermediate precursor; wherein the chlorine intermediate precursor is present in the reactant mixture in an amount of from about 1 part per million (ppm) to about 100 ppm, based on the total volume of the reactant mixture; (b) allowing at least a portion of the reactant mixture to contact at least a portion of the OCM catalyst composition and react via an OCM reaction to form a product mixture comprising unreacted methane and C₂₊ hydrocarbons; wherein the process for producing C₂₊ hydrocarbons is characterized by improved performance in the presence of the chlorine intermediate precursor in the reactant mixture; (c) recovering at least a portion of the product mixture from the OCM reactor; and (d) recovering at least a portion of the C₂₊ hydrocarbons from the product mixture. The OCM catalyst composition can be any suitable OCM catalyst composition, such as a single metal oxide, mixtures of single metal oxides, a mixed metal oxide, mixtures of mixed metal oxides, and the like, or combinations thereof.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

As used herein, the terms “Cₓ hydrocarbons” and “Cₓs” are interchangeable and refer to any hydrocarbon having x number of carbon atoms (C). For example, the terms “C₄ hydrocarbons” and “C₄s” both refer to any hydrocarbons having exactly 4 carbon atoms, such as n-butane, iso-butane, cyclobutane, 1-butene, 2-butene, isobutylene, butadiene, and the like, or combinations thereof.

As used herein, the term “Cₓ₊ hydrocarbons” refers to any hydrocarbon having equal to or greater than x carbon atoms (C). For example, the term “C₂₊ hydrocarbons” refers to any hydrocarbons having 2 or more carbon atoms, such as ethane, ethylene, C₃s, C₄s, C₅s, etc.

In an aspect, a process for producing C₂₊ hydrocarbons (e.g., C₂₊ olefins, C₂₊ alkanes, etc.) as disclosed herein can comprise (A) introducing a reactant mixture (e.g., oxidative coupling of methane (OCM) reactant mixture) to an OCM reactor comprising the OCM catalyst composition as disclosed herein, wherein the reactant mixture comprises methane (CH₄), oxygen (O₂), and a chlorine intermediate precursor; wherein the chlorine intermediate precursor is present in the reactant mixture in an amount of from about 1 part per million (ppm) to about 100 ppm, based on the total volume of the reactant mixture and (B) allowing at least a portion of the reactant mixture to contact at least a portion of the OCM catalyst composition and react via an OCM reaction to form a product mixture comprising unreacted methane and C₂₊ hydrocarbons.

The OCM catalyst composition as disclosed herein can comprise any catalyst suitable for use in an OCM reaction, such as a metal oxide catalyst. The OCM catalyst composition can comprise a single metal oxide, mixtures of single metal oxides, a mixed metal oxide, mixtures of mixed metal oxides, and the like, or combinations thereof; wherein the OCM catalyst composition can be a supported OCM catalyst composition and/or an unsupported OCM catalyst composition as will be described in more detail later herein.

As will be appreciated by one of skill in the art, and with the help of this disclosure, and without wishing to be limited by theory, an OCM catalyst comprising a single metal might not provide all the necessary properties for an optimum OCM reaction (e.g., best OCM reaction outcome) at the best level, and as such conducting an optimum OCM reaction may require an OCM catalyst with tailored composition in terms of metals present, wherein the different metals can have optimum properties for various OCM reaction steps, and wherein the different metals can provide synergistically for achieving the best performance for the OCM catalyst in an OCM reaction.

In aspects where the OCM catalyst composition comprises more than one metal and/or a metal that can have multiple oxidation states within the OCM catalyst composition, and without wishing to be limited by theory, different metals and/or different oxidation states of the same metal present in the OCM catalyst compositions as disclosed herein can display synergetic effects in terms of conversion and selectivity. Further, and without wishing to be limited by theory, different ion radii and valences of different metals that may be present in the OCM catalyst compositions as disclosed herein can generate formation of surface oxygen vacancies (e.g., uncompensated oxygen vacancies), which can lead to further improvement of catalyst performance, for example in terms of conversion, selectivity, stability, etc., as will be discussed in more detail later herein.

Without wishing to be limited by theory, an OCM reaction can propagate by following a mechanism according to reactions (1)-(5):

[O]ₛ+CH₄→[OH]ₛ+CH₃. (1)

2CH₃.→C₂H₆ (2)

CH₃.+O₂↔.CH₃O₂ (3)

CH₃.+[O]ₛ↔[CH₃O]ₛ (4)

2[OH]ₛ+½O₂→2[O]ₛ+H₂O (5)

wherein “s” denotes a species adsorbed onto the catalyst surface. As will be appreciated by one of skill in the art, and with the help of this disclosure, two or more of reactions (1)-(5) can occur concurrently (as opposed to sequentially). According to reaction (1), the activation of methane occurs with the participation of active adsorbed oxygen sites [O]ₛ, leading to the formation of methyl radicals (CH₃.) and adsorbed hydroxyl groups [OH]ₛ. According to reaction (2), the coupling of methyl radicals to form the coupling product ethane (C₂H₆) occurs in gas phase; wherein reaction (2) has a low activation energy, and therefore, does not limit the overall reaction rate. Ethane can be further converted to ethylene through parallel reactions of thermal dehydrogenation and catalytic oxidative dehydrogenation. For example, ethane can further interact with active adsorbed oxygen sites [O]ₛ to form an ethyl radical, where the ethyl radical can lead to the formation of ethylene with the participation of an additional active adsorbed oxygen site [O]ₛ. Ethylene thermal dehydrogenation, as well as the formation of the ethyl radical and the subsequent formation of ethylene have a lower activation energy than the formation of methyl radicals (according to reaction (1)), and thus do not limit the overall reaction rate. According to reaction (3), methyl radicals can react with gas phase oxygen to form an oxygenate product .CH₃O₂. According to reaction (4), methyl radicals can also re-adsorb onto the catalyst surface and react with surface oxygen (e.g., active adsorbed oxygen sites [O]ₛ) to form an adsorbed oxygenate species [CH₃O]ₛ. The oxygenates formed according to reactions (3) and (4) can further form CO and CO₂, and as such the reaction steps according to reactions (3) and (4) are the main reactions controlling the selectivity of various OCM catalysts. In addition to reaction (1), the activity of the catalyst can also be influenced by the removal of the hydroxyl group [OH]ₛ from the catalyst surface according to reaction (5), which re-oxidizes the reduced sites back, and thereby completes the full cycle of the OCM reaction. The adsorbed hydroxyl group [OH]ₛ can undergo a dehydroxylation reaction step (e.g., removal or elimination of the surface adsorbed hydroxyl group from the catalyst), which creates surface oxygen vacancies (e.g., uncompensated oxygen vacancies). The surface oxygen vacancies can react with molecular oxygen and/or the oxygenate product .CH₃O₂ to produce the active adsorbed oxygen sites [O]ₛ, which in turn activates methane and creates more methyl radicals according to reaction (1), thereby improving methane conversion. Decreasing the amount of oxygenate species can further decrease the formation of deep oxidation products, thereby improving selectivity to desired products (e.g., ethylene). The mechanism of OCM reaction is described in more detail in Lomonosov, V. I. and Sinev, M. Y., Kinetics and Catalysis, 2016, vol. 57, pp. 647-676; which is incorporated by reference herein in its entirety.

Further, in order to improve selectivity to desired products (e.g., olefins, ethylene), the process for producing C₂₊ hydrocarbons (e.g., C₂₊ olefins, C₂₊ alkanes, etc.) as disclosed herein can optionally comprise multiple stages (e.g., as part of a multi-stage process).

In some aspects, a process for producing C₂₊ hydrocarbons (e.g., C₂₊ olefins, C₂₊ alkanes, etc.) as disclosed herein can comprise a single stage or multiple stages (e.g., as part of a multi-stage process), wherein each individual stage can comprise an OCM reactor or reaction zone, and wherein each individual stage can be repeated as necessary to achieve a target methane conversion and/or target selectivity (e.g., target C₂₊ selectivity) for the overall multi-stage process. For purposes of the disclosure herein, a stage of a process can be defined as a single pass conversion through a given catalyst bed. Any suitable physical configuration and arrangement of components of a catalyst bed (e.g., catalyst particles, inert media, spacers, support structures, screens, etc.) within the given catalyst bed may be employed. A multi-stage process generally comprises a plurality of individual stages (e.g., a plurality of reaction zones), wherein each individual stage (e.g., reaction zone) comprises a single pass conversion through a given catalyst bed. While the current disclosure will be discussed in detail in the context of a single stage comprising a single reactor comprising a given catalyst bed, it should be understood that any suitable stage/reactor/catalyst bed configurations can be used. For example, two or more stages of a multi-stage process can be housed in one or more reactors. As will be appreciated by one of skill in the art, and with the help of this disclosure, multiple stages can be housed within a single reaction vessel, for example a vessel comprising two or more catalyst beds in series. Further, as will be appreciated by one of skill in the art, and with the help of this disclosure, multiple vessels can be part of a single stage, for example two or more vessels in parallel, wherein a reactant mixture is distributed between and introduced to the two or more vessels in parallel. While the current disclosure will be discussed in detail in the context of a process comprising 1 or 2 stages, it should be understood that any suitable number of stages can be used, such as for example, 1 stage, 2 stages, 3 stages, 4 stages, 5 stages, 6 stages, 7 stages, 8 stages, 9 stages, 10 stages, or more stages. For example, a multi-stage processes may be implemented via a corresponding plurality of reactors in series.

The OCM reactant mixture (e.g., first reactant mixture, second reactant mixture) can be a gaseous mixture. The OCM reactant mixture can comprise a hydrocarbon or mixtures of hydrocarbons, oxygen, and a chlorine intermediate precursor. In some aspects, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons such as diesel fuel, jet fuel, gasoline, tars, kerosene, etc.), oxygenated hydrocarbons, biodiesel, alcohols, dimethyl ether, and the like, or combinations thereof. In an aspect, the OCM reactant mixture can comprise CH₄, O₂, and methyl chloride (CH₃Cl).

In aspects where the process for producing C₂₊ hydrocarbons (e.g., C₂₊ olefins, C₂₊ alkanes, etc.) as disclosed herein comprises multiple stages (e.g., as part of a multi-stage process), and as will be appreciated by one of skill in the art, and with the help of this disclosure, methane (or a hydrocarbon or mixtures of hydrocarbons) can be introduced into a multi-stage process in the first stage into the OCM reactor (e.g., a first reactor); wherein the OCM reactant mixture for subsequent stages (e.g., a second stage) will utilize the unreacted methane and any other hydrocarbons present that were recovered from the first stage (after passing through any other processes that are part of the first stage). In some aspects, additional or supplemental methane (or a hydrocarbon or mixtures of hydrocarbons) could be optionally added to reactant mixtures in stages other than the first stage (e.g., fresh hydrocarbon feed at one or more stages subsequent to a first stage), to supplement a recovered unreacted methane, if necessary.

The O₂ used in the OCM reactant mixture (e.g., first reactant mixture, second reactant mixture) can be oxygen gas (which may be obtained via a membrane separation process), technical oxygen (which may contain some air), air, oxygen enriched air, and the like, or combinations thereof. O₂ can be introduced in the first reactor, as well as in any subsequent stages in any OCM reactor (e.g., a second reactor).

In an aspect, the OCM reactant mixture (e.g., first reactant mixture, second reactant mixture) can be characterized by a CH₄/O₂ molar ratio of from about 3:1 to about 20:1, alternatively from about 4:1 to about 16:1, alternatively from about 4:1 to about 10:1, alternatively from about 4.5:1 to about 9:1, or alternatively from about 5:1 to about 8:1.

In an aspect, the OCM reactant mixture can comprise a chlorine intermediate precursor. Nonlimiting examples of chlorine intermediate precursors suitable for use in the present disclosure include hydrogen chloride, methyl chloride, methylene chloride, chloroform, carbon tetrachloride, chloroethane (ethyl chloride), 1,1-dichloroethane, 1,2-dichloroethane, vinyl chloride, dichloroethene, 1,1-dichloroethylene (vinylidene chloride), cis-1,2-dichloroethylene, 1,2-trans-dichloroethylene, trichloroethylene (TCE), 1,1,1-trichloroethane, 1,1,2-trichloroethane (vinyl trichloride), 1,1,1-trichloroethene, 1,1,1,2-tetrachloroethane, 1,1,2,2-tetrachloroethane (acetylene tetrachloride), tetrachloroethylene (perchloroethylene; PCE), pentachloroethane, hexachloroethane, chloropropane, 1,2-dichloropropane (propylene dichloride), 1-chloro-2-propene, 1,3-cis-dichloro-1-propene, 1,3-trans-dichloropropene, trichloropropane, 1,2,3-trichloropropane, chloroprene, 2-butylene dichloride, hexachlorobutadiene, hexachlorocyclopentadiene, monochlorocyclohexane, monochlorobenzene, and the like, or combinations thereof.

In an aspect, any suitable volatile organic chloride can be used as the chlorine intermediate precursors in the present disclosure. In some aspects, the chlorine intermediate precursor can comprise any suitable organic chlorine compound having from 1 to 3 carbon atoms.

In an aspect, the chlorine intermediate precursor can be selected from the group consisting of hydrogen chloride (HCl), methyl chloride (CH₃Cl), methylene chloride (CH₂Cl₂), chloroform (CHCl₃), carbon tetrachloride (CCl₄), ethyl chloride (C₂H₅Cl), 1,2-dichloroethane (C₂H₄Cl₂), trichloroethylene (C₂HCl₃), and combinations thereof.

In some aspects, the chlorine intermediate precursor can be selected from the group consisting of methyl chloride (CH₃Cl), methylene chloride (CH₂Cl₂), chloroform (CHCl₃), carbon tetrachloride (CCl₄), ethyl chloride (C₂H₅Cl), 1,2-dichloroethane (C₂H₄Cl₂), trichloroethylene (C₂HCl₃), and combinations thereof.

In some aspects, the chlorine intermediate precursor can be introduced continuously to the OCM reactor. For example, the chlorine intermediate precursor can be mixed into the hydrocarbon feed prior to introducing the hydrocarbon feed to the OCM reactor. As another example, the hydrocarbons and the chlorine intermediate precursor can be introduced separately to the OCM reactor. In other aspects, the chlorine intermediate precursor can be introduced discontinuously to the OCM reactor.

In some aspects, the chlorine intermediate precursor can be introduced to the reactor as part of the reactant mixture, e.g., the chlorine intermediate precursor can be added to the reactant mixture (e.g., methane and oxygen) and can be introduced to the reactor with the methane and oxygen via a common stream. In other aspects, the chlorine intermediate precursor can be introduced to the reactor via a stream other than the feed stream for methane and oxygen (e.g., a separate stream).

In an aspect, the chlorine intermediate precursor can be present in the reactant mixture in an amount of from about 1 ppm to about 100 ppm, alternatively from about 1 ppm to about 90 ppm, alternatively from about 1 ppm to about 80 ppm, alternatively from about 1 ppm to about 70 ppm, alternatively from about 1 ppm to about 60 ppm, alternatively from about 1 ppm to about 50 ppm, alternatively from about 2 ppm to about 40 ppm, alternatively from about 3 ppm to about 30 ppm, alternatively from about 4 ppm to about 25 ppm, alternatively from about 5 ppm to about 22.5 ppm, or alternatively from about 10 ppm to about 20 ppm, based on the total volume of the reactant mixture. In some aspects, the chlorine intermediate precursor can be introduced continuously to the reactor. In other aspects, the chlorine intermediate precursor can be introduced discontinuously to the reactor.

In an aspect, the chlorine intermediate precursor can be a chlorine radical precursor, wherein the chlorine intermediate is a chlorine radical. In some aspects, the process of producing C₂₊ hydrocarbons (e.g., C₂₊ olefins, C₂₊ alkanes, etc.) as disclosed herein can comprise (i) allowing at least a portion of the chlorine intermediate precursor to generate a chlorine intermediate (e.g., chlorine radical), and (ii) allowing a portion of the OCM reactants (e.g., CH₄, O₂) to react via the chlorine intermediate. Without wishing to be limited by theory, in the presence of elements with redox properties (e.g., a metal with redox properties, such as Mn, M, a redox rare earth element, etc.), the chlorine intermediate precursor can lead to the formation of a chlorine intermediate, such as a chlorine radical.

In some aspects, the step (B) of allowing at least a portion of the reactant mixture to contact at least a portion of the OCM catalyst composition and react via an OCM reaction to form a product mixture comprising unreacted methane and C₂₊ hydrocarbons can further comprise (i) allowing a first portion of the reactant mixture to react via an OCM reaction, (ii) allowing at least a portion of the chlorine intermediate precursor to generate a chlorine intermediate, and (iii) allowing a second portion of the reactant mixture to react via the chlorine intermediate.

When a chlorine intermediate precursor as disclosed herein is present in a reactant mixture that contacts an OCM catalyst composition as disclosed herein (e.g., while conducting an OCM reaction), the chlorine intermediate precursor can adsorb onto the catalyst surface and can block oxygenate centers, thereby reducing the formation of deep oxidation products (COₓ), such as carbon monoxide (CO) and/or carbon dioxide (CO₂). As will be appreciated by one of skill in the art, and with the help of this disclosure, minimizing the formation of deep oxidation products (COₓ) can lead to an increased yield of C₂₊ hydrocarbons. Furthermore, chlorine intermediates (e.g., sodium chloride, manganese chloride, rare earth element chlorides, etc.) can also block oxygenate centers, thereby reducing the formation of deep oxidation products (COₓ).

Consequently, and without wishing to be limited by theory, the proposed mechanism by which the chlorine intermediates influence the outcome of the OCM reaction renders the effect of the chlorine intermediates on an OCM reaction as independent of the catalyst type used for catalyzing the OCM reaction. In other words, the use of a chlorine intermediate precursor in the feed (e.g., reactant mixture) in a process employing an OCM reaction as disclosed herein has an effect on the process (i.e., improved process performance) that does not depend on the catalyst type used for catalyzing the OCM reaction, i.e., the chlorine intermediate precursor can be used in the reactant mixture in an OCM reaction to improve process performance as disclosed herein with a variety of different OCM catalysts (as opposed to a single particular type of OCM catalyst), as demonstrated throughout the present application.

The stoichiometric reactions of methane conversion in the presence of a chlorine intermediate precursor can be described similar to the case of OCM reaction, as represented by equations (6)-(10) for various chlorine intermediate precursors:

CH₄+½O₂+(HCl)→C₂H₄+H₂O (6)

CH₄+½O₂+(CH₃Cl)→C₂H₄+H₂O (7)

CH₄+½O₂+(CH₂Cl₂)→C₂H₄+H₂O (8)

CH₄+½O₂+(CCl₄)→C₂H₄+H₂O (9)

CH₄+½O₂+(C₂H₅Cl)→C₂H₄+H₂O (10)

Without wishing to be limited by theory, the chlorine intermediate precursors do not get consumed in reactions (6)-(10), due to a radical mechanism of chlorine participation in the reactions that can regenerate the chlorine intermediate precursors. As will be appreciated by one of skill in the art, and with the help of this disclosure, at OCM reaction temperatures (e.g., fairly elevated temperatures, for example equal to or greater than about 700° C.), the chlorine intermediate precursors lead to HCl formation, which in turn reacts according to equation (6).

In an aspect, the chlorine intermediate precursor can generate a chlorine intermediate via contacting at least a portion of the chlorine intermediate precursor with the OCM catalyst (e.g., OCM catalyst composition) to form a chlorinated OCM catalyst (e.g., chlorinated OCM catalyst composition), wherein at least a portion of the chlorinated OCM catalyst can generate the chlorine intermediate. The chlorine intermediate precursor can be contacted with the OCM catalyst either continuously or discontinuously, as disclosed herein. Without wishing to be limited by theory, the metals of the OCM catalyst can react with the chlorine intermediate precursor that was introduced in a gas phase (e.g., with the reactant mixture) to an OCM reactor and can form chlorides on a catalyst surface. For purposes of the disclosure herein, the term “chlorine intermediate precursor” refers to both a chlorine compound introduced in gas phase to the OCM reactor, as well as chloride, oxychloride or any other chlorine-containing compound adsorbed and/or formed on a surface of the OCM catalyst (e.g., chlorinated OCM catalyst), wherein such chloride or any other chlorine-containing compound adsorbed and/or formed on a surface of the OCM catalyst can further generate a chlorine intermediate, such as a chlorine radical. For purposes of the disclosure herein, the terms “chlorinated OCM catalyst” and/or “chlorinated OCM catalyst composition” refers to an OCM catalyst having a chloride, oxychloride or any other chlorine-containing compound adsorbed and/or formed on a surface of the OCM catalyst, wherein such chloride, oxychloride or any other chlorine-containing compound adsorbed and/or formed on a surface of the OCM catalyst (e.g., chlorinated OCM catalyst) can further generate a chlorine intermediate, such as a chlorine radical. The chlorinated OCM catalyst can comprise an oxychloride of a metal such as a rare earth element oxychloride, lanthanum oxychloride, cerium oxychloride, manganese oxychloride, tin oxychloride, antimony oxychloride, lead oxychloride, bismuth oxychloride, iron oxychloride, molybdenum oxychloride, tantalum oxychloride, niobium oxychloride, rhenium oxychloride, and the like, or combinations thereof. In an aspect, the chlorinated OCM catalysts can comprise redox metal oxychlorides that promote the formation of chlorine radicals.

Without wishing to be limited by theory, the chloride, oxychloride or any other chlorine-containing compound adsorbed and/or formed on the catalyst surface (e.g., chlorinated OCM catalyst) can further generate the chlorine intermediate (e.g., chlorine radical), for example via a redox agent, such as a redox metal, Mn, a rare earth element, etc. For purposes of the disclosure herein, a chemical species that has redox properties, can also be referred to as a “redox agent.” A redox agent generally refers to a chemical species that possesses the ability to undergo both an oxidation reaction and a reduction reaction, and such ability usually resides in the chemical species having more than one stable oxidation state other than the oxidation state of zero (0). Redox agents (e.g., a redox element; a redox metal or a metal with redox properties; Mn; Ce; etc.) can generally convert between oxide forms and chloride forms, which can lead to the formation of chlorine intermediates, thus promoting methane conversion reactions via chlorine intermediates. Further, and without wishing to be limited by theory, the chloride, oxychloride or any other chlorine-containing compound adsorbed and/or formed on the catalyst surface can react with oxygen centers on the chlorinated OCM catalyst (e.g., on the catalyst surface) to generate the chlorine intermediate (e.g., chlorine radical), for example by reducing such oxygen centers on the catalyst while oxidizing a chloride and/or oxychloride to a chlorine radical. Further, and without wishing to be limited by theory, chlorides, oxychlorides or any other chlorine-containing compound adsorbed and/or formed on the chlorinated OCM catalyst surface can decrease the amount of oxygen available for deep oxidation reactions (e.g., by reducing oxygen centers), thereby minimizing deep oxidation reactions, for example deep oxidation reactions of methane to carbon dioxide.

For example, when the catalyst comprises Mn, such as in the form of manganese oxides (e.g., MnO₂), and when the chlorine intermediate precursor comprises HCl, the generation of chlorine radicals (Cl.) can be represented by equations (11) and (12):

MnO₂+4HCl ⇄MnCl₂+Cl₂+2H₂O (11)

Cl₂⇄2Cl. (12)

Without wishing to be limited by theory, the chlorine intermediate (e.g., chlorine radical, Cl.) can diffuse away from the catalyst surface (e.g., into the gas phase) and can initiate the formation of methyl radicals, ethyl radicals, and ultimately the formation of ethylene molecules. The chlorine intermediate (e.g., chlorine radical, Cl.) can re-generate the HCl while forming various alkyl radicals (e.g., methyl radicals (CH₃.), ethyl radicals (C₂H₅.), etc.), and such HCl can re-initiate the steps of forming the chlorine radical by interacting with the catalyst (e.g., for example according to reactions (11) and (12)); and/or in gas phase, for example as represented by equations (13)-(16):

CH₄+Cl. [symbol] CH₃.+HCl (13)

2CH₃. [symbol] C₂H₆ (14)

C₂H₆+Cl. [symbol] C₂H₅.+HCl (15)

C₂Hₛ. [symbol] C₂H₄+H. (16)

As disclosed herein, in the presence of chlorine intermediate precursors in the reactant mixture, (A) the oxidative conversion reactions (e.g., OCM reactions, for example as represented by equations (I)-(II), (1)-(5)) and (B) reactions via a chlorine intermediate (e.g., chlorine radical reactions, for example as represented by equations (11)-(16)) can occur simultaneously.

The OCM reactant mixture (e.g., first reactant mixture, second reactant mixture) can further comprise a diluent. A diluent can be introduced in the first reactor, as well as in any subsequent stages in any OCM reactor (e.g., a second reactor). The diluent is inert with respect to the OCM reaction, e.g., the diluent does not participate in the OCM reaction. In an aspect, the diluent can comprise water (e.g., steam), nitrogen, inert gases, and the like, or combinations thereof. In an aspect, the diluent can be present in the OCM reactant mixture in an amount of from about 0.5% to about 80%, alternatively from about 5% to about 50%, or alternatively from about 10% to about 30%, based on the total volume of the OCM reactant mixture.

In some aspects, the diluent comprises steam. Steam can be present in the reactant mixture in an amount of from about 5% to about 70%, alternatively from about 10% to about 60%, or alternatively from about 15% to about 50%, based on the total volume of the reactant mixture.

In an aspect, the reactor (e.g., OCM reactor) can comprise an adiabatic reactor, an autothermal reactor, an isothermal reactor, a tubular reactor, a cooled tubular reactor, a continuous flow reactor, a fixed bed reactor, a fluidized bed reactor, a moving bed reactor, and the like, or combinations thereof. In an aspect, the OCM reactor can comprise a catalyst bed comprising the OCM catalyst composition as disclosed herein.

The OCM reactor can be operated under adiabatic conditions, non-adiabatic conditions, autothermal conditions, isothermal conditions, etc. For purposes of the disclosure herein, the term “non-adiabatic conditions” refers to process conditions wherein a reactor is subjected to external heat exchange or transfer (e.g., the reactor is heated; or the reactor is cooled), which can be direct heat exchange and/or indirect heat exchange. As will be appreciated by one of skill in the art, and with the help of this disclosure, the terms “direct heat exchange” and “indirect heat exchange” are known to one of skill in the art. By contrast, the term “adiabatic conditions” refers to process conditions wherein a reactor is not subjected to external heat exchange (e.g., the reactor is not heated; or the reactor is not cooled). Generally, external heat exchange implies an external heat exchange system (e.g., a cooling system; a heating system) that requires energy input and/or output. As will be appreciated by one of skill in the art, and with the help of this disclosure, external heat transfer can also result from heat loss from the catalyst bed (or reactor) owing to radiation heat transfer, conduction heat transfer, convection heat transfer, and the like, or combinations thereof. For example, the catalyst bed can participate in heat exchange with the external environment, and/or with reactor zones upstream and/or downstream of the catalyst bed.

In some aspects, the OCM reactor (e.g., the first reactor, the second reactor) can be an adiabatic reactor. In some aspects, the OCM reactor (e.g., the first reactor, the second reactor) can be an autothermal reactor. The OCM reactors can be fixed bed reactors, such as axial flow reactors, or radial flow reactors. As will be appreciated by one of skill in the art, and with the help of this disclosure, certain fixed bed reactors, such as radial flow reactors, can decrease a reactor pressure drop, which may in turn increase a desired selectivity. In some aspects, the reactor can comprise a catalyst bed comprising the OCM catalyst composition disclosed herein.

In some aspects, the OCM reactor (e.g., the first reactor, the second reactor) can be characterized by any suitable OCM reactor operational parameters, such as temperature (e.g., feed preheat temperature or reactor inlet temperature, reactor effluent temperature, etc.), pressure, flow rate (e.g., space velocity), and the like, or combinations thereof.

In an aspect, the OCM reactant mixture (e.g., first reactant mixture, second reactant mixture) can be characterized by an inlet temperature (e.g., can be introduced to the OCM reactor at an inlet temperature) of from about 150° C. to about 900° C., alternatively from about 300° C. to about 900° C., alternatively from about 225° C. to about 850° C., alternatively from about 250° C. to about 800° C., alternatively from about 550° C. to about 800° C., alternatively from about 575° C. to about 750° C., or alternatively from about 600° C. to about 700° C. As will be appreciated by one of skill in the art, and with the help of this disclosure, while the OCM reaction is exothermic, heat input is necessary for promoting the formation of methyl radicals from CH₄, as the C—H bonds of CH₄ are very stable, and the formation of methyl radicals from CH₄ is endothermic. In an aspect, the OCM reactant mixture can be introduced to the OCM reactor at a temperature effective to promote an OCM reaction.

Without wishing to be limited by theory, when the temperature (e.g., reactor temperature) reaches values of above about 900° C., thermal reactions (e.g., methane steam reforming) will start to make significant contribution to the overall reaction, which in turn will result in lower C₂₊ selectivity. As will be appreciated by one of skill in the art, and with the help of this disclosure, since the upper limit of the operating temperature is dictated by a significant contribution of thermal reactions, a broad temperature rise in an OCM reactor performing an OCM reaction can be achieved by lowering the inlet temperature; which in turn requires for the OCM catalyst to display high C₂₊ selectivity at low reactor temperatures (e.g., less than about 700° C.). However, and without wishing to be limited by theory, at relatively low reactor temperatures, the selectivity that can be achieved is limited by the gas-phase methyl radical deep oxidation reactions; which are more significant at lower reaction temperatures.

In some aspects, the OCM reactor can be characterized by a reactor effluent temperature of from about 700° C. to about 1,000° C., alternatively from about 700° C. to about 975° C., alternatively from about 700° C. to about 950° C., alternatively from about 700° C. to about 925° C., alternatively from about 750° C. to about 925° C., alternatively from about 800° C. to about 925° C., alternatively from about 800° C. to about 900° C., alternatively from about 800° C. to about 875° C., or alternatively from about 820° C. to about 850° C.

In some aspects, a process for producing C₂₊ hydrocarbons (e.g., C₂₊ olefins, C₂₊ alkanes, etc.) as disclosed herein can comprise a single stage or multiple stages (e.g., as part of a multi-stage process), wherein each individual stage can comprise an oxidative coupling of methane (OCM) reactor or reaction zone, and wherein each individual stage can be repeated as necessary to achieve a target methane conversion for the overall multi-stage process.

In an aspect, a process for producing C₂₊ hydrocarbons (e.g., C₂₊ olefins, C₂₊ alkanes, etc.) can comprise a first stage and a second stage, wherein the first stage comprises a first reactor (e.g., first autothermal reactor, first adiabatic reactor, first OCM reactor), and wherein the second stage comprises a second reactor (e.g., second autothermal reactor, a second adiabatic reactor, a second OCM reactor), and wherein the first reactor and the second reactor are in series, with the second reactor downstream of the first reactor.

In some aspects, a process for producing C₂₊ hydrocarbons (e.g., C₂₊ olefins, C₂₊ alkanes, etc.) as disclosed herein can be a multi-stage process, wherein the multi-stage process further comprises one or more additional stages downstream of a first stage and/or a second stage (with each successive downstream stage having a corresponding OCM reactor in series with and downstream of an immediately preceding stage/reactor), as necessary to achieve a target methane conversion and/or a target C₂₊ selectivity for the overall multi-stage process. Each additional stage can comprise (i) introducing a reactant mixture to a reactor comprising an OCM catalyst composition as disclosed herein, wherein the reactant mixture comprises CH₄, O₂, and the chlorine intermediate precursor, and wherein the reactant mixture is characterized by an inlet temperature; (ii) allowing at least a portion of the reactant mixture to contact at least a portion of the OCM catalyst composition and react via an OCM reaction to form a product mixture comprising unreacted methane and C₂₊ hydrocarbons (e.g., C₂₊ olefins, C₂₊ alkanes, etc.); (iii) recovering at least a portion of the product mixture from the reactor, wherein the product mixture is characterized by an outlet temperature, which may be equal to or greater than about the inlet temperature; and (iv) optionally cooling the product mixture (e.g., via a heat exchanger). In some aspects, the reactant mixture can comprise at least a portion of an upstream product mixture recovered from an upstream reactor. In some aspects, the reactant mixture can further comprise at least a portion of a downstream product mixture recovered from a downstream reactor. For purposes of the disclosure herein, all descriptions related to the first stage (such as descriptions of first OCM reactor, first OCM catalyst composition, first reactant mixture (e.g., OCM reactant mixture), first product mixture (e.g., OCM product mixture), first inlet temperature, first outlet temperature, etc.) can be applied to the corresponding components of any subsequent stage (such as descriptions of reactor (e.g., OCM reactor), OCM catalyst composition, reactant mixture (e.g., OCM reactant mixture), product mixture (e.g., OCM product mixture), inlet temperature, outlet temperature, etc., respectively), unless otherwise specified herein.

The OCM reactor (e.g., the first reactor, the second reactor) can be characterized by a pressure of from about ambient pressure (e.g., atmospheric pressure) to about 500 psig, alternatively from about ambient pressure to about 200 psig, or alternatively from about ambient pressure to about 150 psig. In an aspect, the process for producing C₂₊ hydrocarbons (e.g., C₂₊ olefins, C₂₊ alkanes, etc.) as disclosed herein can be carried out at ambient pressure.

The OCM reactor (e.g., the first reactor, the second reactor) can be characterized by a gas hourly space velocity (GHSV) of from about 500 h⁻¹ to about 10,000,000 h⁻¹, alternatively from about 500 h⁻¹ to about 1,000,000 h⁻¹, alternatively from about 500 h⁻¹ to about 500,000 h⁻¹, alternatively from about 500 h⁻¹ to about 100,000 h⁻¹, alternatively from about 500 h⁻¹ to about 50,000 h⁻¹, alternatively from about 1,000 h⁻¹ to about 500,000 h⁻¹, alternatively from about 1,000 h⁻¹ to about 40,000 h⁻¹, or alternatively from about 1,500 h⁻¹ to about 25,000 h⁻¹, alternatively from about 1,500 h⁻¹ to about 500,000 h⁻¹, alternatively from about 2,000 h⁻¹ to about 500,000 h⁻¹, alternatively from about 5,000 h⁻¹ to about 500,000 h⁻¹, alternatively from about 10,000 h⁻¹ to about 500,000 h⁻¹, or alternatively from about 50,000 h⁻¹ to about 500,000 h⁻¹. Generally, the GHSV relates a reactant (e.g., reactant mixture) gas flow rate to a reactor volume. GHSV is usually measured at standard temperature and pressure.

The OCM reactor (e.g., the first reactor, the second reactor) can be characterized by a residence time of from about 10 milliseconds (ms) to about 2 seconds (s), alternatively from about 100 ms to about 1.5 s, alternatively from about 500 ms to about 1.5 s, alternatively from about 500 ms to about 1 s, alternatively less than about 500 ms, alternatively less than about 100 ms, alternatively less than about 50 ms, alternatively less than about 25 ms, alternatively less than about 10 ms, alternatively less than about 5 ms, alternatively less than about 4 ms, alternatively less than about 3 ms, alternatively less than about 2 ms, or alternatively less than about 1 ms. Generally, the residence time of reactor refers to the average amount of time that a compound (e.g., a molecule of that compound) spends in that particular reactor, reaction zone and/or catalyst bed thereof, e.g., the average amount of time that it takes for a compound (e.g., a molecule of that compound) to travel through the reactor, reaction zone and/or catalyst bed thereof. The residence time may also refer to the contact time between the gaseous reactant mixture and the catalyst bed.

In an aspect, the OCM reactor can be characterized by any suitable reactor temperature and/or catalyst bed temperature. The OCM reactor (e.g., the first reactor, the second reactor) can be characterized by a temperature of from about 400° C. to about 1,000° C., alternatively from about 500° C. to about 950° C., or alternatively from about 600° C. to about 900° C. For example, the OCM reactor can be characterized by a reactor temperature and/or catalyst bed temperature of equal to or greater than about 300° C., alternatively equal to or greater than about 600° C., alternatively equal to or greater than about 700° C., alternatively equal to or greater than about 750° C., alternatively equal to or greater than about 800° C., alternatively equal to or greater than about 850° C., alternatively from about 300° C. to about 1,600° C., alternatively from about 600° C. to about 1,400° C., alternatively from about 600° C. to about 1,300° C., alternatively from about 700° C. to about 1,200° C., alternatively from about 750° C. to about 1,150° C., alternatively from about 800° C. to about 1,125° C., or alternatively from about 850° C. to about 1,100° C.

In an aspect, the OCM reaction can be characterized by an OCM catalyst ignition temperature, defined as the temperature at which the oxygen conversion is 50%. In such aspect, the process can further comprise (prior to the step (A) of introducing to the reactor a reactant mixture comprising CH₄, O₂, and the chlorine intermediate precursor) (i) introducing to the reactor an activating mixture comprising CH₄ and O₂ and excluding the chlorine intermediate precursor until the reactor attains an activation temperature, wherein the activation temperature is defined as the temperature at which the oxygen conversion is substantially 100%, and (ii) maintaining the reactor at the activation temperature for an activation time period, wherein the activation temperature is above the OCM catalyst ignition temperature. The term “activation temperature” refers to the temperature at which the reactor is operated during the activation time period. In an aspect, the activation temperature can be equal to or greater than about 775° C., alternatively equal to or greater than about 800° C., alternatively equal to or greater than about 825° C., alternatively from about 800° C. to about 850° C., or alternatively equal to or greater than about 850° C. In some aspects, as further detailed hereinbelow, a mixture such as the activation mixture absent the chlorine intermediate precursor can be introduced into the reactor during activation of the OCM catalyst composition, and the chlorine intermediate precursor can be introduced into the OCM reactor via the reactant mixture only subsequent the activation time at the activation temperature. That is, subsequent activation, a reactant mixture comprising methane, oxygen, and the chlorine intermediate precursor can be introduced into the OCM reactor for a reaction time at an OCM reaction temperature.

In some aspects, the chlorine intermediate precursor can be introduced continuously to the reactor. In other aspects, the chlorine intermediate precursor can be introduced discontinuously to the reactor. In aspects where the chlorine intermediate precursor is introduced discontinuously to the reactor, the process for producing ethylene as disclosed herein can comprise (1) introducing an activation mixture comprising CH₄ and O₂ and excluding the chlorine intermediate precursor to the reactor for an activation time period; (2) introducing a reactant mixture comprising CH₄ and O₂ and including the chlorine intermediate precursor to the reactor for a reaction time period; and (3) repeating steps (1) and (2) as necessary to achieve a target methane conversion and/or a target ethylene selectivity. As will be appreciated by one of skill in the art, and with the help of this disclosure, at the very beginning of the process for producing ethylene as disclosed herein, an activation period may need to occur, to activate the catalyst. The activation time period is usually followed by a reaction time period. Further, and as will be appreciated by one of skill in the art, and with the help of this disclosure, the activation mixture is a reactant mixture lacking the chlorine intermediate precursor, wherein the activation mixture being introduced to the reactor results in the production of ethylene, although with a conversion and/or selectivity that are generally lower than a desired or target conversion and/or selectivity, respectively.

Generally, a conversion of a reagent or reactant refers to the percentage (usually mol %) of reagent that reacted to both undesired and desired products, based on the total amount (e.g., moles) of reagent present before any reaction took place. For purposes of the disclosure herein, the conversion of a reagent is a percent (%) conversion based on moles converted. As will be appreciated by one of skill in the art, and with the help of this disclosure, the reactant mixture in OCM reactions is generally characterized by a methane to oxygen molar ratio of greater than 1:1, and as such the O₂ conversion is fairly high in OCM processes, most often approaching 90% to 100%. Without wishing to be limited by theory, oxygen is usually a limiting reagent in OCM processes. The oxygen conversion can be calculated by using equation (17):

$${O}_{2} conversion = \frac{{O}_{2}^{m} - {O}_{2}^{o u t}}{{O}_{2}^{in}} \times 100 \% \qquad ( 17 )$$

wherein O₂ⁱⁿ=number of moles of O₂ that entered the OCM reactor as part of the reactant mixture (or activation mixture); and O₂^{out}=number of moles of O₂ that was recovered from the OCM reactor as part of the product mixture.

In an aspect, the activation time period can be, for example, from about 10 minutes to about 6 hours. The reaction time period can be, for example, from about 1 day to about 2 days. The activation temperature is greater than the ignition temperature, and can be equal to or greater than the OCM reaction temperature. For example, in some aspects, a difference between the activation temperature and the OCM reaction temperature can be equal to or greater than about 5° C., 10° C., 15° C., 20° C., or 25° C., i.e., the OCM reaction temperature is at least about 5° C., 10° C., 15° C., 20° C., or 25° C. lower than the activation temperature. In some aspects, the activation temperature can be equal to or greater than about 800° C., and the OCM reaction temperature can be less than about 800° C. In some aspects, the reaction temperature can be the temperature of the catalyst bed in the OCM reactor during the reaction time, or a feed inlet temperature of the reactant mixture introduced into the OCM reactor during the reaction time. Similarly, in some aspects, the activation temperature can be the temperature of the catalyst bed in the OCM reactor during the activation time, or a feed inlet temperature of the activation mixture introduced into the OCM reactor during the activation time.

In some aspects, the activation temperature can be substantially equal to the OCM reaction temperature.

In other aspects, the OCM reaction temperature can be above the OCM catalyst ignition temperature and below the activation temperature, and the process can further comprise reducing the temperature of the OCM reactor to the OCM reaction temperature from the activation temperature prior to (a) introducing the reactant mixture to the reactor comprising the OCM catalyst composition, wherein the reactant mixture comprises methane (CH₄), oxygen (O₂), and the chlorine intermediate precursor.

In an aspect, the O₂ conversion (e.g., in each stage) of the OCM as disclosed herein can be equal to or greater than about 90%, alternatively equal to or greater than about 95%, alternatively equal to or greater than about 99%, alternatively equal to or greater than about 99.9%, or alternatively about 100%. The OCM reaction temperature can thus be a temperature which provides for such O₂ conversion. For example, in some aspects, the OCM reaction temperature can be a temperature at which the oxygen conversion is equal to or greater than about 90%.

In an aspect, the process for producing C₂₊ hydrocarbons (e.g., C₂₊ olefins, C₂₊ alkanes, etc.) as disclosed herein can be characterized by a reaction temperature (e.g., OCM reaction temperature) effective for achieving an O₂ conversion of equal to or greater than about 90% that is decreased by equal to or greater than about 20° C., alternatively by equal to or greater than about 30° C., alternatively by equal to or greater than about 40° C., or alternatively by equal to or greater than about 50° C., when compared to a reaction temperature effective for achieving an O₂ conversion of equal to or greater than about 90% of an otherwise similar process absent the chlorine intermediate precursor in the reactant mixture. In an aspect, such an OCM reaction temperature can be in the range of from about 600° C. to about 900° C., alternatively from about 650° C. to about 850° C., alternatively from about 700° C. to about 900° C., or alternatively equal to or greater than about 600° C., 650° C., 700° C., 750° C., or 800° C.

In an aspect, the process for producing C₂₊ hydrocarbons (e.g., C₂₊ olefins, C₂₊ alkanes, etc.) as disclosed herein can be characterized by a reaction temperature effective for achieving an O₂ conversion of equal to or greater than about 90% of less than about 700° C., alternatively less than about 750° C., alternatively less than about 800° C., alternatively less than about 825° C., or alternatively less than about 850° C. As will be appreciated by one of skill in the art, and with the help of this disclosure, the reaction temperature effective for achieving an O₂ conversion of equal to or greater than about 90% is dependent upon specific reactor conditions, such as for example methane to oxygen molar ratio, type and size of reactor, GHSV, etc.

In an aspect, the process for producing C₂₊ hydrocarbons (e.g., C₂₊ olefins, C₂₊ alkanes, etc.) as disclosed herein can be characterized by improved performance in the presence of the chlorine intermediate precursor in the reactant mixture; e.g., improved performance when the chlorine intermediate precursor is present in the reactant mixture (as opposed to the chlorine intermediate precursor being absent from the reactant mixture). For purposes of the disclosure herein, the improved performance of the process is defined by the process having at least one modification (e.g., process modification) selected from the group consisting of increased C₂₊ selectivity, decreased selectivity to carbon dioxide (CO₂), increased catalyst activity, increased methane conversion, increased catalyst stability, increased catalyst life-time, decreased reaction temperature effective for achieving an O₂ conversion of equal to or greater than about 90%, and combinations thereof; wherein the catalyst activity is defined as the O₂ conversion under a set of given OCM reactor operational parameters; wherein the catalyst stability is defined as a catalyst activity variation within about ±10% of a target catalyst activity over a time period of equal to or greater than about 50 hours (h), alternatively equal to or greater than about 100 h, alternatively equal to or greater than about 250 h, alternatively equal to or greater than about 500 h, alternatively equal to or greater than about 1,000 h, or alternatively equal to or greater than about 5,000 h; and wherein the target catalyst activity is defined as a target O₂ conversion of equal to or greater than about 90% under the same set of given OCM reactor operational parameters. For purposes of the disclosure herein, the improved performance of the process for producing C₂₊ hydrocarbons (e.g., C₂₊ olefins, C₂₊ alkanes, etc.) as disclosed herein is defined as being relative to an otherwise similar process that employs a reactant mixture comprising CH₄ and O₂, without the chlorine intermediate precursor. For example, the improved performance of the process for producing C₂₊ hydrocarbons (e.g., C₂₊ olefins, C₂₊ alkanes, etc.) as disclosed herein can comprise an increased C₂₊ selectivity, wherein the C₂₊ selectivity of the process as disclosed herein is increased when compared to a C₂₊ selectivity of an otherwise similar process that employs a reactant mixture comprising CH₄ and O₂, without the chlorine intermediate precursor. As another example, the improved performance of the process for producing C₂₊ hydrocarbons (e.g., C₂₊ olefins, C₂₊ alkanes, etc.) as disclosed herein can comprise an increased catalyst activity, wherein the catalyst activity in the process as disclosed herein is increased when compared to a catalyst activity in an otherwise similar process that employs a reactant mixture comprising CH₄ and O₂, without the chlorine intermediate precursor. As will be appreciated by one of skill in the art, and with the help of this disclosure, the term “otherwise similar process” refers to a process that employs all the process parameters and variables as the process for producing C₂₊ hydrocarbons (e.g., C₂₊ olefins, C₂₊ alkanes, etc.) as disclosed herein (e.g., same reactor, same catalyst, same reactor inlet temperature, same reactor pressure, same GHSV, same methane to oxygen ratio, etc.), except for the chlorine intermediate precursor which is not introduced to the reactor in the otherwise similar process.

In an aspect, the process for producing C₂₊ hydrocarbons (e.g., C₂₊ olefins, C₂₊ alkanes, etc.) employing a chlorine intermediate precursor in the reactant mixture as disclosed herein can be characterized by a catalyst activity (i.e., O₂ conversion under a set of given OCM reactor operational parameters) that is increased by equal to or greater than about 1%, alternatively equal to or greater than about 2.5%, alternatively equal to or greater than about 5%, alternatively equal to or greater than about 10%, alternatively equal to or greater than about 15%, or alternatively equal to or greater than about 20% when compared to a catalyst activity (i.e., O₂ conversion under the same set of given OCM reactor operational parameters) in an otherwise similar process that employs a reactant mixture comprising CH₄ and O₂, without the chlorine intermediate precursor.

In an aspect, the process for producing C₂₊ hydrocarbons (e.g., C₂₊ olefins, C₂₊ alkanes, etc.) as disclosed herein can be characterized by a catalyst activity variation within about ±10%, alternatively within about ±9%, alternatively within about ±8%, alternatively within about ±7%, alternatively within about ±6%, alternatively within about ±5%, alternatively within about ±4%, alternatively within about ±3%, alternatively within about ±2%, or alternatively within about ±1% of a target catalyst activity over a time period of equal to or greater than about 50 hours (h), alternatively equal to or greater than about 100 h, alternatively equal to or greater than about 250 h, alternatively equal to or greater than about 500 h, alternatively equal to or greater than about 1,000 h, or alternatively equal to or greater than about 5,000 h, wherein the catalyst activity is defined as the O₂ conversion under a set of given OCM reactor operational parameters, and wherein the target catalyst activity is defined as a target O₂ conversion of equal to or greater than about 90% under the same set of given OCM reactor operational parameters. In some aspects, the target O₂ conversion can be equal to or greater than about 90%, alternatively equal to or greater than about 95%, alternatively equal to or greater than about 99%, alternatively equal to or greater than about 99.9%, or alternatively about 100%.

In an aspect, the process for producing C₂₊ hydrocarbons (e.g., C₂₊ olefins, C₂₊ alkanes, etc.) as disclosed herein can be characterized by an increased catalyst stability when compared to a catalyst stability in an otherwise similar process that employs a reactant mixture comprising CH₄ and O₂, without the chlorine intermediate precursor. For example, the process for producing C₂₊ hydrocarbons (e.g., C₂₊ olefins, C₂₊ alkanes, etc.) as disclosed herein can be characterized by a catalyst activity variation over a given time period that is decreased when compared with a catalyst activity variation over the same time period in an otherwise similar process that employs a reactant mixture comprising CH₄ and O₂, without the chlorine intermediate precursor.

In an aspect, the process for producing C₂₊ hydrocarbons (e.g., C₂₊ olefins, C₂₊ alkanes, etc.) as disclosed herein can be characterized by a catalyst activity variation over a given time period that is decreased by equal to or greater than about 1%, alternatively equal to or greater than about 5%, alternatively equal to or greater than about 10%, alternatively equal to or greater than about 15%, alternatively equal to or greater than about 20%, alternatively equal to or greater than about 25%, alternatively equal to or greater than about 30%, alternatively equal to or greater than about 35%, alternatively equal to or greater than about 45%, alternatively equal to or greater than about 50%, alternatively equal to or greater than about 60%, or alternatively equal to or greater than about 75% when compared with a catalyst activity variation over the same time period in an otherwise similar process that employs a reactant mixture comprising CH₄ and O₂, without the chlorine intermediate precursor.

In an aspect, the process for producing C₂₊ hydrocarbons (e.g., C₂₊ olefins, C₂₊ alkanes, etc.) as disclosed herein can be characterized by an increased catalyst life-time when compared to a catalyst life-time in an otherwise similar process that employs a reactant mixture comprising CH₄ and O₂, without the chlorine intermediate precursor. For purposes of the disclosure herein, the term “catalyst life-time” is defined as the time period that the catalyst displays a catalyst activity variation within about +10%, alternatively within about ±9%, alternatively within about ±8%, alternatively within about ±7%, alternatively within about ±6%, alternatively within about ±5%, alternatively within about ±4%, alternatively within about ±3%, alternatively within about ±2%, or alternatively within about ±1% of a target catalyst activity.

In an aspect, the process for producing C₂₊ hydrocarbons (e.g., C₂₊ olefins, C₂₊ alkanes, etc.) as disclosed herein can be characterized by a catalyst activity variation within about ±10% of a target catalyst activity over a first time period (e.g., first catalyst life-time); wherein an otherwise similar process that employs a reactant mixture comprising CH₄ and O₂, without the chlorine intermediate precursor, is characterized by a catalyst activity variation within about ±10% of the same target catalyst activity over a second time period (e.g., second catalyst life-time); and wherein the first time period is increased when compared to the second time period. For example, the first time period (e.g., first catalyst life-time) can be increased by equal to or greater than about 1%, alternatively equal to or greater than about 5%, alternatively equal to or greater than about 10%, alternatively equal to or greater than about 15%, alternatively equal to or greater than about 20%, alternatively equal to or greater than about 25%, alternatively equal to or greater than about 30%, alternatively equal to or greater than about 35%, alternatively equal to or greater than about 45%, alternatively equal to or greater than about 50%, alternatively equal to or greater than about 60%, or alternatively equal to or greater than about 75% when compared to the second time period (e.g., second catalyst life-time). In some aspects, the second time period can be less than about 50 h.

In some aspects, the process for producing C₂₊ hydrocarbons (e.g., C₂₊ olefins, C₂₊ alkanes, etc.) as disclosed herein can be characterized by a methane conversion that is about equal to a methane conversion of an otherwise similar process conducted with a reactant mixture comprising methane and oxygen and excluding the chlorine intermediate precursor. In such aspects, the same level of methane conversion can be achieved with a reduced production of deep oxidation products (e.g., CO, CO₂) and/or an increased selectivity to C₂₊ hydrocarbons (e.g., increased selectivity to C₂ hydrocarbons).

In other aspects, the process for producing C₂₊ hydrocarbons (e.g., C₂₊ olefins, C₂₊ alkanes, etc.) as disclosed herein can be characterized by a methane conversion that increased when compared to a methane conversion of an otherwise similar process conducted with a reactant mixture comprising methane and oxygen and excluding the chlorine intermediate precursor. For example, the process for producing C₂₊ hydrocarbons (e.g., C₂₊ olefins, C₂₊ alkanes, etc.) as disclosed herein can be characterized by a methane conversion that is increased by equal to or greater than about 1%, 2%, 3%, 4%, 5%, 6%, 7%, 8%, 9%, or 10%, alternatively from about 6% to about 22%, or alternatively from about 10% to about 20%, when compared to a methane conversion of an otherwise similar process conducted with a reactant mixture comprising methane and oxygen and excluding the chlorine intermediate precursor. In such aspects, the same level of methane conversion can be achieved with a reduced production of deep oxidation products (e.g., CO, CO₂) and/or an increased selectivity to C₂₊ hydrocarbons (e.g., increased selectivity to C₂ hydrocarbons). In such aspects, the process for producing C₂₊ hydrocarbons (e.g., C₂₊ olefins, C₂₊ alkanes, etc.) as disclosed herein can be characterized by a selectivity to C₂₊ hydrocarbons (e.g., selectivity to C₂ hydrocarbons) that is increased when compared to a selectivity to C₂₊ hydrocarbons (e.g., selectivity to C₂ hydrocarbons) of an otherwise similar process conducted with a reactant mixture comprising methane and oxygen and excluding the chlorine intermediate precursor.

In some aspects, an amount of unreacted methane and/or an amount of ethylene in the product mixture can be periodically monitored to determine methane conversion and/or ethylene selectivity, respectively. As will be appreciated by one of skill in the art, and with the help of this disclosure, the methane conversion and the ethylene selectivity correlate with the activity of the catalyst and with the activity of the catalyst with respect to producing a desired product (e.g., ethylene), respectively.

Generally, a selectivity to a desired product or products (e.g., ethylene selectivity) refers to how much desired product (e.g., ethylene) was formed divided by the total products formed, both desired and undesired (e.g., ethylene, ethane, etc.). For purposes of the disclosure herein, the selectivity to a desired product is a percent (%) selectivity based on moles converted into the desired product. Further, for purposes of the disclosure herein, a Cₓ selectivity (e.g., C₂ selectivity, C₂₊ selectivity, etc.) can be calculated by dividing a number of moles of carbon (C) from CH₄ that were converted into the desired product (e.g., C_{C2H4}, C_{C2H6}, etc.) by the total number of moles of C from CH₄ that were converted (e.g., C_{C2H4}, C_{C2H6}, C_{C2H2}, C_{C3H6}, C_{C3H8}, C_{C4s}, C_{CO2}, C_{CO}, etc.). Thus, C_{C2H4}=number of moles of C from CH₄ that were converted into C₂H₄; C_{C2H6}=number of moles of C from CH₄ that were converted into C₂H₆; C_{C2H2}=number of moles of C from CH₄ that were converted into C₂H₂; C_{C3H6}=number of moles of C from CH₄ that were converted into C₃H₆; C_{C3H8}=number of moles of C from CH₄ that were converted into C₃H₈; C_{C4s}=number of moles of C from CH₄ that were converted into C₄ hydrocarbons (C₄s); C_{CO2}=number of moles of C from CH₄ that were converted into CO₂; C_{CO}=number of moles of C from CH₄ that were converted into CO; etc.

A C₂₊ selectivity (e.g., selectivity to C₂₊ hydrocarbons) refers to how much C₂H₄, C₃H₆, C₂H₂, C₂H₆, C₃H₈, and C₄s were formed divided by the total products formed, including C₂H₄, C₃H₆, C₂H₂, C₂H₆, C₃H₈, C₄s, CO₂ and CO. For example, the C₂₊ selectivity can be calculated by using equation (18):

$${C}_{2 +} selectivity = \frac{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4 s}}\end{matrix}}{\begin{matrix}2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + \\ 3 {C}_{{C}_{3} {H}_{8}} + 4 {C}_{{C}_{4 s}} + {C}_{{CO}_{2}} + {C}_{CO}\end{matrix}} \times 1 \; 00 \% \qquad ( 18 )$$

As will be appreciated by one of skill in the art, and with the help of this disclosure, if a specific product and/or hydrocarbon product is not produced in a certain OCM reaction/process, then the corresponding C_{Cx} is 0, and the term is simply removed from selectivity calculations.

Without wishing to be limited by theory, when the selectivity (e.g., C₂₊ selectivity) of an OCM process increases, less methane is converted to undesirable products, such as deep oxidation products (e.g., CO, CO₂), which in turn means that more oxygen (which is often the limiting reagent in OCM processes) is available for the conversion of methane to desirable products (e.g., C₂ products, C₂H₄, C₂₊ products, etc.), thus enabling an increased yield of desired C₂₊ products. As will be appreciated by one of skill in the art, and with the help of this disclosure, the higher the temperature in the reactor, the more deep oxidation products will be produced, and as such lower temperatures for achieving an O₂ conversion of equal to or greater than about 90% will lead to a lower amount of deep oxidation products produced, thus resulting in the increased methane conversion to the desirable products.

In some aspects, the process for producing C₂₊ hydrocarbons (e.g., C₂₊ olefins, C₂₊ alkanes, etc.) as disclosed herein can be characterized by a C₂₊ selectivity (e.g., a selectivity to hydrocarbons comprising two or more carbons) of equal to or greater than about 70%, alternatively equal to or greater than about 75%, alternatively equal to or greater than about 80%, or alternatively equal to or greater than about 85% at a given reaction temperature in the OCM reactor (e.g., in a single stage), wherein the given reaction temperature can be equal to or greater than about 750° C., 760° C., or 775° C. In such aspects, the process for producing C₂₊ hydrocarbons (e.g., C₂₊ olefins, C₂₊ alkanes, etc.) as disclosed herein can be characterized by a C₂₊ selectivity at a temperature in the range of from about 600° C. to about 900° C., alternatively from about 650° C. to about 850° C., alternatively from about 700° C. to about 900° C., or alternatively equal to or greater than about 600° C., 650° C., 700° C., 750° C., 600° C. to 850° C. that is increased when compared to a C₂₊ selectivity of an otherwise similar process lacking the chlorine intermediate precursor in the reactant mixture.

In some aspects, the process for producing C₂₊ hydrocarbons (e.g., C₂₊ olefins, C₂₊ alkanes, etc.) as disclosed herein can be characterized by a C₂₊ selectivity (selectivity to hydrocarbons comprising two or more carbons) at the OCM reaction temperature that is increased when compared to a C₂₊ selectivity of an otherwise similar process lacking the chlorine intermediate precursor in the reactant mixture. In such aspects, the process for producing C₂₊ hydrocarbons (e.g., C₂₊ olefins, C₂₊ alkanes, etc.) as disclosed herein can be characterized by a C₂₊ selectivity (selectivity to hydrocarbons comprising two or more carbons) at the OCM reaction temperature that is increased by equal to or greater than about 1%, 2%, 3%, 4%, 5%, 6%, 7%, 8%, 9%, 10%, 12.5%, or 15%, alternatively from about 1% to about 10%, alternatively from about 2% to about 6%, or alternatively from about 3% to about 5%, when compared to a C₂₊ selectivity of an otherwise similar process lacking the chlorine intermediate precursor in the reactant mixture.

In some aspects, the process for producing C₂₊ hydrocarbons (e.g., C₂₊ olefins, C₂₊ alkanes, etc.) as disclosed herein can be characterized by a C₂ selectivity (selectivity to hydrocarbons comprising two carbons or selectivity to C₂ hydrocarbons) at the OCM reaction temperature that is increased when compared to a C₂ selectivity of an otherwise similar process lacking the chlorine intermediate precursor in the reactant mixture. In such aspects, the process for producing C₂₊ hydrocarbons (e.g., C₂₊ olefins, C₂₊ alkanes, etc.) as disclosed herein can be characterized by a C₂ selectivity (selectivity to hydrocarbons comprising two carbons or selectivity to C₂ hydrocarbons) at the OCM reaction temperature that is increased by equal to or greater than about 1%, 2%, 3%, 4%, 5%, 6%, 7%, 8%, 9%, 10%, 12.5%, or 15%, alternatively from about 1% to about 10%, alternatively from about 2% to about 6%, or alternatively from about 3% to about 5%, when compared to a C₂ selectivity of an otherwise similar process lacking the chlorine intermediate precursor in the reactant mixture.

In an aspect, the process for producing C₂₊ hydrocarbons (e.g., C₂₊ olefins, C₂₊ alkanes, etc.) as disclosed herein can further comprise minimizing deep oxidation of methane to COₓ products, such as carbon monoxide (CO) and/or carbon dioxide (CO₂). Without wishing to be limited by theory, when the selectivity to desired products (e.g., C₂₊ selectivity, C₂₌ selectivity (selectivity to ethylene (C₂H₄))) of an OCM process increases, less methane is converted to undesirable products, such as deep oxidation products (e.g., CO, CO₂), which in turn means that more oxygen (which is often the limiting reagent in OCM processes) is available for the conversion of methane to desirable products (e.g., C₂ products, C₂H₄, C₂₊ products, etc.), thus enabling an increased yield of desired C₂₊ products. In some aspects, and as will be appreciated by one of skill in the art, and with the help of this disclosure, CO can be recovered as a useful product from the product mixture, wherein CO can be further used, for example as part of syngas.

In some aspects, the product mixture can comprise less than about 15 mol %, alternatively less than about 14 mol %, alternatively less than about 13 mol %, alternatively less than about 12 mol %, alternatively less than about 11 mol %, alternatively less than about 10 mol %, alternatively less than about 9 mol %, alternatively less than about 8 mol %, alternatively less than about 7 mol %, alternatively less than about 6 mol %, or alternatively less than about 5 mol % carbon monoxide (CO) and/or carbon dioxide (CO₂).

In an aspect, the process for producing C₂₊ hydrocarbons (e.g., C₂₊ olefins, C₂₊ alkanes, etc.) as disclosed herein can be characterized by (1) a C_{CO2} selectivity (selectivity to CO₂) at the OCM reaction temperature that is decreased when compared to a C_{CO2} selectivity of an otherwise similar process lacking the chlorine intermediate precursor in the reactant mixture; and/or (2) a CH₄ conversion at the OCM reaction temperature that is increased when compared to a CH₄ conversion of an otherwise similar process lacking the chlorine intermediate precursor in the reactant mixture. In some aspects, the process for producing C₂₊ hydrocarbons (e.g., C₂₊ olefins, C₂₊ alkanes, etc.) as disclosed herein can be characterized by (1) a C_{CO2} selectivity at the OCM reaction temperature that is decreased by equal to or greater than about 2%, 3%, 4%, 5%, 6%, 7%, 8%, 9%, or 10%, alternatively from about 1% to about 6%, or alternatively from about 3% to about 5%, when compared to a C_{CO2} selectivity of an otherwise similar process lacking the chlorine intermediate precursor in the reactant mixture.

In an aspect, the process for producing C₂₊ hydrocarbons (e.g., C₂₊ olefins, C₂₊ alkanes, etc.) as disclosed herein can comprise recovering at least a portion of the product mixture (e.g., first product mixture, second product mixture) from the OCM reactor (e.g., OCM reactor, first OCM reactor, second OCM reactor), wherein the product mixture can comprise C₂₊ hydrocarbons (e.g., C₂₊ olefins, C₂₊ alkanes, etc.), water, CO, CO₂, and unreacted methane. In an aspect, the process for producing C₂₊ hydrocarbons (e.g., C₂₊ olefins, C₂₊ alkanes, etc.) as disclosed herein can comprise recovering at least a portion of the C₂₊ hydrocarbons (e.g., C₂₊ olefins, C₂₊ alkanes, etc.) from the product mixture (e.g., first product mixture, second product mixture). The product mixture can comprise C₂₊ hydrocarbons (including olefins), unreacted methane, and optionally a diluent. The C₂₊ hydrocarbons can comprise C₂ hydrocarbons and C₃ hydrocarbons. In an aspect, the C₂₊ hydrocarbons can further comprise C₄ hydrocarbons (C₄s), such as for example butane, iso-butane, n-butane, butylene, etc. The C₂ hydrocarbons can comprise ethylene (C₂H₄) and ethane (C₂H₆). The C₂ hydrocarbons can further comprise acetylene (C₂H₂). The C₃ hydrocarbons can comprise propylene (C₃H₆) and propane (C₃H₈).

In some aspects, a method for producing C₂₊ hydrocarbons (e.g., C₂₊ olefins, C₂₊ alkanes, etc.) as disclosed herein can comprise recovering at least a portion of the C₂₊ hydrocarbons from the product mixture. For purposes of the disclosure herein, the term “recovering at least a portion of the C₂₊ hydrocarbons from the product mixture” can refer to isolating C₂₊ hydrocarbons from the product mixture and/or purifying C₂₊ hydrocarbons from the product mixture, as well as to employing the recovered C₂₊ hydrocarbons (e.g., recovered C₂₊ olefins, recovered C₂₊ alkanes, etc.) in a downstream process, for example a polyolefin production process (e.g., polyethylene production process), ethylene oxide production process, etc. For example, the term “recovering at least a portion of ethylene from the product mixture” can refer to isolating ethylene from the product mixture and/or purifying ethylene from the product mixture, as well as to employing the recovered ethylene in a downstream process, for example a polyethylene production process, ethylene oxide production process, etc.

The water produced from the OCM reaction and the water used as a diluent (if water diluent is used) can be separated from the product mixture prior to separating any of the other product mixture components. For example, by cooling down the product mixture to a temperature where the water condenses (e.g., below 100° C. at ambient pressure), the water can be removed from the product mixture, by using a flash chamber for example.

In some aspects, at least a portion of the C₂₊ hydrocarbons can be separated (e.g., recovered) from the product mixture to yield recovered C₂₊ hydrocarbons. The C₂₊ hydrocarbons can be separated from the product mixture by using any suitable separation technique. In an aspect, at least a portion of the C₂₊ hydrocarbons can be separated from the product mixture by distillation (e.g., cryogenic distillation).

In an aspect, at least a portion of the recovered C₂₊ hydrocarbons can be used for ethylene production. In an aspect, at least a portion of ethylene can be separated from the product mixture (e.g., from the C₂₊ hydrocarbons, from the recovered C₂₊ hydrocarbons) to yield recovered ethylene and recovered hydrocarbons, by using any suitable separation technique (e.g., distillation, cryogenic distillation). In an aspect, at least a portion of the recovered hydrocarbons (e.g., recovered C₂₊ hydrocarbons after olefin separation, such as separation of C₂H₄ and C₃H₆) can be converted to ethylene, for example by a conventional steam cracking process.

A process for producing C₂₊ hydrocarbons (e.g., C₂₊ olefins, C₂₊ alkanes, etc.) as disclosed herein can comprise recovering at least a portion of the C₂₊ olefins from the product mixture. In an aspect, at least a portion of the C₂₊ olefins can be separated from the product mixture by distillation (e.g., cryogenic distillation). As will be appreciated by one of skill in the art, and with the help of this disclosure, the C₂₊ olefins are generally individually separated from their paraffin counterparts by distillation (e.g., cryogenic distillation). For example, ethylene can be separated from ethane by distillation (e.g., cryogenic distillation). As another example, propylene can be separated from propane by distillation (e.g., cryogenic distillation).

In an aspect, at least a portion of the unreacted methane can be separated from the product mixture to yield recovered methane. Methane can be separated from the product mixture by using any suitable separation technique, such as for example distillation (e.g., cryogenic distillation). At least a portion of the recovered methane (e.g., unreacted methane) can be recycled to the reactant mixture.

In an aspect, the OCM reactor can comprise any suitable OCM catalyst composition as disclosed herein, for example any catalyst suitable for use in an OCM reaction, such as a metal oxide catalyst. The OCM catalyst composition as disclosed herein can comprise a single metal oxide, mixtures of single metal oxides, a mixed metal oxide, mixtures of mixed metal oxides, and the like, or combinations thereof; as will be described in more detail later herein. The OCM catalyst compositions suitable for use in the present disclosure can be supported OCM catalyst compositions and/or unsupported OCM catalyst compositions.

In some aspects, the supported OCM catalyst compositions as disclosed herein can comprise a support, wherein the support can be catalytically active (e.g., the support can catalyze an OCM reaction, such as MgO). In other aspects, the supported OCM catalyst compositions can comprise a support, wherein the support can be catalytically inactive (e.g., the support cannot catalyze an OCM reaction, such as SiO₂). In yet other aspects, the supported OCM catalyst compositions can comprise a catalytically active support and a catalytically inactive support. Nonlimiting examples of a support suitable for use in the present disclosure include MgO, Al₂O₃, SiO₂, ZrO₂, TiO₂, and the like, or combinations thereof. As will be appreciated by one of skill in the art, and with the help of this disclosure, the support can be purchased or can be prepared by using any suitable methodology, such as for example precipitation/co-precipitation, sol-gel techniques, templates/surface derivatized metal oxides synthesis, solid-state synthesis of mixed metal oxides, microemulsion techniques, solvothermal techniques, sonochemical techniques, combustion synthesis, etc.

In some aspects, the OCM catalyst composition can further comprise a support, wherein at least a portion of the OCM catalyst composition contacts, coats, is embedded in, is supported by, and/or is distributed throughout at least a portion of the support. In such aspects, the support can be in the form of powders, particles, pellets, monoliths, foams, honeycombs, and the like, or combinations thereof. Nonlimiting examples of support particle shapes include cylindrical, discoidal, spherical, tabular, ellipsoidal, equant, irregular, cubic, acicular, and the like, or combinations thereof.

In some aspects, the OCM catalyst composition can further comprise a porous support. As will be appreciated by one of skill in the art, and with the help of this disclosure, a porous material (e.g., support) can provide for an enhanced surface area of contact between the OCM catalyst composition and the reactant mixture, which in turn would result in a higher CH₄ conversion to CH₃..

In some aspects, the OCM catalyst composition of this disclosure is an unsupported OCM catalyst composition comprising no support.

In an aspect, the OCM catalyst compositions as disclosed herein can be prepared by using any suitable methodology.

In an aspect, the OCM catalyst composition as disclosed herein can be characterized by the general formula Aₐ₁Z_{b1}E_{c1}D_{d1}Oₓ₁, wherein A is an alkaline earth metal; wherein Z is a first rare earth element; wherein E is a second rare earth element; wherein D is a redox agent or a third rare earth element; wherein the first rare earth element, the second rare earth element, and the third rare earth element, when present, are not the same; wherein a1 is 1.0; wherein b1 is from about 0.1 to about 10.0, alternatively from about 0.3 to about 10.0, alternatively from about 0.5 to about 8, or alternatively from about 1 to about 5; wherein c1 is from about 0 to about 10.0, alternatively from about 0.1 to about 10, alternatively from about 0.1 to about 8, or alternatively from about 0.5 to about 5; wherein d1 is from about 0 to about 10.0, alternatively from about 0.1 to about 8, or alternatively from about 0.5 to about 5; and wherein x1 balances the oxidation states. As will be appreciated by one of the skill in the art, and with the help of this disclosure, each of the A, Z, E and D can have multiple oxidation states within the OCM catalyst composition, and as such x1 can have any suitable value that allows for the oxygen anions to balance all the cations. Without wishing to be limited by theory, the different metals (A, Z, E, and D) present in the OCM catalyst composition as disclosed herein display synergetic effects in terms of conversion and selectivity. Further, and without wishing to be limited by theory, different ion radii and valences of the multiple metals (A, Z, E, and D) present in the OCM catalyst composition as disclosed herein can generate formation of uncompensated oxygen vacancies, which can lead to further improvement of catalyst performance, for example in terms of conversion, selectivity, stability, etc.

The OCM catalyst composition characterized by the general formula Aₐ₁Z_{b1}E_{c1}D_{d1}Oₓ₁ as disclosed herein can comprise an alkaline earth metal (A). The alkaline earth metal (A) can be selected from the group consisting of magnesium (Mg), calcium (Ca), strontium (Sr), barium (Ba), and combinations thereof. In an aspect, the alkaline earth metal (A) is strontium (Sr).

The OCM catalyst composition characterized by the general formula Aₐ₁Z_{b1}E_{c1}D_{d1}Oₓ₁ as disclosed herein can comprise a first rare earth element (Z), wherein the first rare earth element (Z) can be selected from the group consisting of lanthanum (La), scandium (Sc), cerium (Ce), praseodymium (Pr), neodymium (Nd), promethium (Pm), samarium (Sm), europium (Eu), gadolinium (Gd), yttrium (Y), terbium (Tb), dysprosium (Dy), holmium (Ho), erbium (Er), thulium (Tm), ytterbium (Yb), lutetium (Lu), and combinations thereof. In an aspect, the first rare earth element (Z) is La. As will be appreciated by one of skill in the art, and with the help of this disclosure, in some aspects, the first rare earth element (Z) can comprise a single rare earth element, such as La. Further, and as will be appreciated by one of skill in the art, and with the help of this disclosure, in some aspects, the first rare earth element (Z) can comprise two or more rare earth elements, such as La, and Nd, for example; or La, Nd, and Pm, as another example; etc.

The OCM catalyst composition characterized by the general formula Aₐ₁Z_{b1}E_{c1}D_{d1}Oₓ₁ as disclosed herein can comprise a second rare earth element (E) and/or a third rare earth element (D), wherein E and D are different. The second rare earth element (E) and the third rare earth element (D) can each independently be selected from the group consisting of lanthanum (La), scandium (Sc), cerium (Ce), praseodymium (Pr), neodymium (Nd), promethium (Pm), samarium (Sm), europium (Eu), gadolinium (Gd), yttrium (Y), terbium (Tb), dysprosium (Dy), holmium (Ho), erbium (Er), thulium (Tm), ytterbium (Yb), lutetium (Lu), and combinations thereof.

As will be appreciated by one of skill in the art, and with the help of this disclosure, in some aspects, the second rare earth element (E) can comprise a single rare earth element, such as Yb. Further, and as will be appreciated by one of skill in the art, and with the help of this disclosure, in some aspects, the second rare earth element (E) can comprise two or more rare earth elements, such as Yb, and Nd, for example; or Yb, and Tm, as another example; or Yb, Nd, and Tm, as yet another example; etc.

As will be appreciated by one of skill in the art, and with the help of this disclosure, in some aspects, the third rare earth element (D) can comprise a single rare earth element, such as Nd. Further, and as will be appreciated by one of skill in the art, and with the help of this disclosure, in some aspects, the third rare earth element (D) can comprise two or more rare earth elements, such as Yb, and Nd, for example; or Yb, Nd, and Pm, as another example; etc.

The OCM catalyst composition characterized by the general formula Aₐ₁Z_{b1}E_{c1}D_{d1}Oₓ₁ as disclosed herein can comprise component (D). As will be appreciated by one of skill in the art, and with the help of this disclosure, D can be either a redox agent or a third rare earth element.

The redox agent (D) can be selected from the group consisting of manganese (Mn), tungsten (W), bismuth (Bi), antimony (Sb), tin (Sn), cerium (Ce), praseodymium (Pr), and combinations thereof. A redox agent generally refers to a chemical species that possesses the ability to undergo both an oxidation reaction and a reduction reaction, and such ability usually resides in the chemical species having more than one stable oxidation state other than the oxidation state of zero (0). As will be appreciated by one of skill in the art, and with the help of this disclosure, some rare earth elements, such as Ce and Pr, can also be considered redox agents. Further, as will be appreciated by one of skill in the art, and with the help of this disclosure, when D is Ce and/or Pr, D can be considered either a redox agent or a third rare earth element.

As will be appreciated by one of skill in the art, and with the help of this disclosure, in some aspects, the redox agent (D) can comprise a single element, such as Mn. Further, and as will be appreciated by one of skill in the art, and with the help of this disclosure, in some aspects, the redox agent (D) can comprise two or more compounds, such as Mn, and W, for example; or Mn, W, and Pr, as another example; etc. In some aspects, the redox agent (D) is Mn. In other aspects, the redox agent (D) is W. In an aspect, the redox agent (D) excludes a rare earth element.

In some aspects, the redox agent can comprise manganese (Mn), tin (Sn), bismuth (Bi), tungsten (W), or combinations thereof. In some aspects, the redox agent can be present in the OCM catalyst composition in an amount of from about 1 wt. % to about 25 wt. %, based on the total weight of the OCM catalyst composition.

In an aspect, the second rare earth element (E) and/or the third rare earth element (D) can be basic (e.g., can exhibit some degree of basicity; can have affinity for hydrogen; can exhibit some degree of affinity for hydrogen). Nonlimiting examples of rare earth elements that can be considered basic for purposes of the disclosure herein include one or more compounds selected from the group consisting of Sc, Ce, Pr, Nd, Pm, Sm, Eu, Gd, Y, Tb, Dy, Ho, Er, Tm, Yb, Lu, and combinations thereof. As will be appreciated by one of skill in the art, and with the help of this disclosure, and without wishing to be limited by theory, the OCM reaction is a multi-step reaction, wherein each step of the OCM reaction could benefit from specific OCM catalytic properties. For example, and without wishing to be limited by theory, an OCM catalyst (e.g., OCM catalyst composition as disclosed herein) should exhibit some degree of basicity to abstract a hydrogen from CH₄ to form hydroxyl groups [OH] on the catalyst surface, as well as methyl radicals (CH₃.). Further, and without wishing to be limited by theory, an OCM catalyst should exhibit oxidative properties for the OCM catalyst to convert the hydroxyl groups [OH] from the OCM catalyst surface to water, which can allow for the OCM reaction to continue (e.g., propagate). Furthermore, as will be appreciated by one of skill in the art, and with the help of this disclosure, and without wishing to be limited by theory, an OCM catalyst could also benefit from properties like oxygen ion conductivity and proton conductivity, which properties can be critical for the OCM reaction to proceed at a very high rate (e.g., its highest possible rate). Furthermore, as will be appreciated by one of skill in the art, and with the help of this disclosure, and without wishing to be limited by theory, an OCM catalyst comprising a single metal might not provide all the necessary properties for an optimum OCM reaction (e.g., best OCM reaction outcome) at the best level, and as such conducting an optimum OCM reaction may require an OCM catalyst with a tailored composition in terms of metals present, as disclosed herein.

In an aspect, the OCM catalyst composition characterized by the general formula Aₐ₁Z_{b1}E_{c1}D_{d1}Oₓ₁ as disclosed herein can comprise one or more oxides of A; one or more oxides of Z; one or more oxides of E; one or more oxides of D; or combinations thereof. The OCM catalyst composition characterized by the general formula Aₐ₁Z_{b1}E_{c1}D_{d1}Oₓ₁ as disclosed herein can comprise one or more oxides of a metal, wherein the metal comprises A, Z, and optionally E and/or D. In some aspects, the OCM catalyst composition characterized by the general formula Aₐ₁Z_{b1}E_{c1}D_{d1}Oₓ₁ as disclosed herein can comprise, consist of, or consist essentially of the one or more oxides of a metal, wherein the metal comprises A, Z, and optionally E and/or D; for example in the case of a unsupported OCM catalyst composition.

In an aspect, the one or more oxides can be present in the OCM catalyst composition characterized by the general formula Aₐ₁Z_{b1}E_{c1}D_{d1}Oₓ₁ as disclosed herein in an amount of from about 0.01 wt. % to about 100.0 wt. %, alternatively from about 0.1 wt. % to about 99.0 wt. %, alternatively from about 1.0 wt. % to about 95.0 wt. %, alternatively from about 10.0 wt. % to about 90.0 wt. %, or alternatively from about 30.0 wt. % to about 70.0 wt. %, based on the total weight of the OCM catalyst composition. As will be appreciated by one of skill in the art, and with the help of this disclosure, a portion of the one or more oxides, in the presence of water, such as atmospheric moisture, can convert to hydroxides, and it is possible that the OCM catalyst composition will comprise some hydroxides, due to exposing the OCM catalyst composition comprising the one or more oxides to water (e.g., atmospheric moisture). Further, as will be appreciated by one of skill in the art, and with the help of this disclosure, a portion of the one or more oxides, in the presence of carbon dioxide, such as atmospheric carbon dioxide, can convert to carbonates, and it is possible that the OCM catalyst composition will comprise some carbonates, due to exposing the OCM catalyst composition comprising the one or more oxides to carbon dioxide (e.g., atmospheric carbon dioxide).

The one or more oxides can comprise a single metal oxide, mixtures of single metal oxides, a mixed metal oxide, mixtures of mixed metal oxides, mixtures of single metal oxides and mixed metal oxides, or combinations thereof.

The single metal oxide comprises one metal selected from the group consisting of A, Z, E, and D. A single metal oxide can be characterized by the general formula MtₘO_{y}; wherein Mt is the metal selected from the group consisting of A, Z, E, and D; and wherein m and y are integers from 1 to 7, alternatively from 1 to 5, or alternatively from 1 to 3. A single metal oxide contains one and only one metal cation. Nonlimiting examples of single metal oxides suitable for use in the OCM catalyst composition characterized by the general formula Aₐ₁Z_{b1}E_{c1}D_{d1}Oₓ₁ as disclosed herein include CaO, MgO, SrO, BaO, La₂O₃, Sc₂O₃, Y₂O₃, CeO₂, Ce₂O₃, Pr₂O₃, PrO₂, Nd₂O₃, Pm₂O₃, Sm₂O₃, Eu₂O₃, Gd₂O₃, Tb₂O₃, Dy₂O₃, Ho₂O₃, Er₂O₃, Lu₂O₃, Yb₂O₃, Tm₂O₃, WO₃, MnO₂, W₂O₃, SnO₂, and the like, or combinations thereof.

In an aspect, mixtures of single metal oxides can comprise two or more different single metal oxides, wherein the two or more different single metal oxides have been mixed together to form the mixture of single metal oxides. Mixtures of single metal oxides can comprise two or more different single metal oxides, wherein each single metal oxide can be selected from the group consisting of CaO, MgO, SrO, BaO, La₂O₃, Sc₂O₃, Y₂O₃, CeO₂, Ce₂O₃, Pr₂O₃, PrO₂, Nd₂O₃, Pm₂O₃, Sm₂O₃, Eu₂O₃, Gd₂O₃, Tb₂O₃, Dy₂O₃, Ho₂O₃, Er₂O₃, Lu₂O₃, Yb₂O₃, Tm₂O₃, WO₃, MnO₂, W₂O₃, and SnO₂. Nonlimiting examples of mixtures of single metal oxides suitable for use in the OCM catalyst composition characterized by the general formula Aₐ₁Z_{b1}E_{c1}D_{d1}Oₓ₁ as disclosed herein include SrO—La₂O₃, SrO—MgO—La₂O₃, SrO—Yb₂O₃—La₂O₃, SrO—Er₂O₃—La₂O₃, SrO—CeO₂—La₂O₃, SrO—MnO₂—La₂O₃, SrO—WO₃—W₂O₃—La₂O₃, SrO—WO₃—Tm₂O₃—La₂O₃, SrO—WO₃—Tm₂O₃—La₂O₃, SrO—BaO—CeO₂—Er₂O₃—La₂O₃, SrO—CeO₂—Ce₂O₃—Er₂O₃—La₂O₃, SrO—BaO—WO₃—W₂O₃—La₂O₃, SrO—BaO—Sm₂O₃—WO₃—W₂O₃—La₂O₃, SrO—MgO—CeO₂—Ce₂O₃—WO₃—W₂O₃—La₂O₃, SrO—CaO—PrO₂—Pr₂O₃—MnO—Mn₂O₃—La₂O₃, and the like, or combinations thereof.

The mixed metal oxide comprises two or more different metals, wherein each metal can be independently selected from the group consisting of A, Z, E, and D. A mixed metal oxide can be characterized by the general formula Mt¹ₘ₁Mt²ₘ₂O_{y}; wherein Mt¹ and Mt² are metals; wherein each of the Mt¹ and Mt² can be independently selected from the group consisting of A, Z, E, and D; and wherein m1, m2 and y are integers from 1 to 15, alternatively from 1 to 10, or alternatively from 1 to 7. In some aspects, Mt¹ and Mt² can be metal cations of different chemical elements, for example Mt¹ can be a lanthanum cation and Mt² can be a strontium cation. In other aspects, Mt¹ and Mt² can be different cations of the same chemical element, wherein Mt¹ and Mt² can have different oxidation states. For example, the mixed metal oxide can comprise Mn₃O₄, wherein Mt¹ can be a Mn (II) cation and Mt² can be a Mn (III) cation. Nonlimiting examples of mixed metal oxides suitable for use in the OCM catalyst composition characterized by the general formula Aₐ₁Z_{b1}E_{c1}D_{d1}Oₓ₁ as disclosed herein include La/SrO; LaYbO₃; SrYb₂O₄; Sr₂CeO₄; Mn₃O₄; La/MgO; Sm₂Ce₂O₇; Er₂Ce₂O₇; CaTm₂O₄; MgYb₂O₄; SrCe_{(1-y)}Yb_{y}O₃, wherein y can be from about 0.01 to about 0.99; and the like; or combinations thereof.

In an aspect, mixtures of mixed metal oxides can comprise two or more different mixed metal oxides, wherein the two or more different mixed metal oxides have been mixed together to form the mixture of mixed metal oxides. Mixtures of mixed metal oxides can comprise two or more different mixed metal oxides, such as La/SrO; LaYbO₃; SrYb₂O₄; Sr₂CeO₄; Mn₃O₄; La/MgO; Sm₂Ce₂O₇; Er₂Ce₂O₇; CaTm₂O₄; MgYb₂O₄; SrCe_{(1-y)}Yb_{y}O₃, wherein y can be from about 0.01 to about 0.99; and the like; or combinations thereof.

In an aspect, mixtures of single metal oxides and mixed metal oxides can comprise at least one single metal oxide and at least one mixed metal oxide, wherein the at least one single metal oxide and the at least one mixed metal oxide have been mixed together to form the mixture of single metal oxides and mixed metal oxides.

In some aspects, the OCM catalyst composition characterized by the general formula Aₐ₁Z_{b1}E_{c1}D_{d1}Oₓ₁ as disclosed herein can comprise a support, wherein at least a portion of the OCM catalyst composition contacts, coats, is embedded in, is supported by, and/or is distributed throughout at least a portion of the support.

In other aspects, the OCM catalyst composition characterized by the general formula Aₐ₁Z_{b1}E_{c1}D_{d1}Oₓ₁ as disclosed herein is a unsupported OCM catalyst composition.

In an aspect, the OCM catalyst composition can be characterized by the general formula Srₐ₁La_{b1}E_{c1}D_{d1}Oₓ₁; wherein E is a second rare earth element other than lanthanum (La); wherein D is a third rare earth element other than lanthanum (La); wherein the second rare earth element and the third rare earth element are different; wherein a1 is 1.0; wherein b1 is from about 0.1 to about 10.0, alternatively from about 0.3 to about 10.0, alternatively from about 0.6 to about 1.2, or alternatively from about 0.8 to about 1.0; wherein c1 is from about 0.01 to about 10.0, alternatively from about 0.4 to about 1, or alternatively from about 0.6 to about 0.8; wherein d1 is from about 0.01 to about 10.0, alternatively from about 0.05 to about 0.15, or alternatively from about 0.09 to about 0.12; and wherein x1 balances the oxidation states.

In some aspects, the OCM catalyst composition characterized by the general formula Srₐ₁La_{b1}E_{c1}D_{d1}Oₓ₁ as disclosed herein can comprise a support, wherein at least a portion of the OCM catalyst composition contacts, coats, is embedded in, is supported by, and/or is distributed throughout at least a portion of the support.

In other aspects, the OCM catalyst composition characterized by the general formula Srₐ₁La_{b1}E_{c1}D_{d1}Oₓ₁ as disclosed herein is a unsupported OCM catalyst composition.

In an aspect, the OCM catalyst composition can be characterized by the general formula Srₐ₁La_{b1}Yb_{c1}Nd_{d1}Oₓ₁; wherein a1 is 1.0; wherein b1 is from about 0.1 to about 10.0, alternatively from about 0.3 to about 10.0, alternatively from about 0.6 to about 1.2, or alternatively from about 0.8 to about 1.0; wherein c1 is from about 0.01 to about 10.0, alternatively from about 0.4 to about 1, or alternatively from about 0.6 to about 0.8; wherein d1 is from about 0.01 to about 10.0, alternatively from about 0.05 to about 0.15, or alternatively from about 0.09 to about 0.12; and wherein x1 balances the oxidation states.

In an aspect, the OCM catalyst composition can be characterized by the general formula Srₐ₁La_{b1}Yb_{c1}Nd_{d1}Oₓ₁; wherein a1 is 1.0; wherein b1 is from about 0.3 to about 10.0; wherein c1 is from about 0.05 to about 10.0; wherein d1 is from about 0.05 to about 10.0; and wherein x1 balances the oxidation states.

In some aspects, the OCM catalyst composition characterized by the general formula Srₐ₁La_{b1}Yb_{c1}Nd_{d1}Oₓ₁ as disclosed herein can comprise a support, wherein at least a portion of the OCM catalyst composition contacts, coats, is embedded in, is supported by, and/or is distributed throughout at least a portion of the support. In such aspects, the support can comprise alumina, wherein the OCM catalyst composition characterized by the general formula Srₐ₁La_{b1}Yb_{c1}Nd_{d1}Oₓ₁/alumina.

In other aspects, the OCM catalyst composition characterized by the general formula Srₐ₁La_{b1}Yb_{c1}Nd_{d1}Oₓ₁ as disclosed herein is a unsupported OCM catalyst composition.

In an aspect, the OCM catalyst composition can be characterized by the general formula Srₐ₁La_{b1}Oₓ₁; wherein a1 is 1.0; wherein b1 is from about 0.1 to about 10.0, alternatively from about 0.3 to about 10.0, alternatively from about 0.5 to about 8, or alternatively from about 1 to about 5; and wherein x1 balances the oxidation states.

In some aspects, the OCM catalyst composition characterized by the general formula Srₐ₁La_{b1}Oₓ₁ as disclosed herein can comprise a support, wherein at least a portion of the OCM catalyst composition contacts, coats, is embedded in, is supported by, and/or is distributed throughout at least a portion of the support. In such aspects, the support can comprise MgO, wherein the OCM catalyst composition characterized by the general formula Srₐ₁La_{b1}Oₓ₁/MgO (Sr—La—MgO).

In other aspects, the OCM catalyst composition characterized by the general formula Srₐ₁La_{b1}Oₓ₁ as disclosed herein is a unsupported OCM catalyst composition.

In an aspect, the OCM catalyst composition as disclosed herein can be characterized by the general formula Laₐ₂Ce_{b2}Oₓ₂ (Laₐ₂—Ce_{b2}—Oₓ₂); wherein a2 is 1.0; wherein b2 is from about 0.3 to about 10.0, alternatively from about 0.05 to about 1.0, alternatively from about 0.06 to about 0.08, or alternatively from about 0.07 to about 0.1; and wherein x2 balances the oxidation states.

In an aspect, the OCM catalyst composition characterized by the general formula Laₐ₂Ce_{b2}Oₓ₂ as disclosed herein can further comprise an optional redox agent, for example a redox agent (D) as described herein for the OCM catalyst composition characterized by the general formula Aₐ₁Z_{b1}E_{c1}D_{d1}Oₓ₁. For example, the redox agent (D) of the OCM catalyst composition characterized by the general formula Laₐ₂Ce_{b2}Oₓ₂ as disclosed herein can comprise manganese (Mn), tungsten (W), bismuth (Bi), antimony (Sb), tin (Sn), cerium (Ce), praseodymium (Pr), or combinations thereof. For example, the OCM catalyst composition characterized by the general formula Laₐ₂Ce_{b2}Oₓ₂ as disclosed herein can have the general formula Laₐ₂Ce_{b2}D_{d2}Oₓ₂; wherein a2 is 1.0; wherein b2 is from about 0.3 to about 10.0, alternatively from about 0.05 to about 1.0, alternatively from about 0.06 to about 0.08, or alternatively from about 0.07 to about 0.1; wherein ds is from about 0 to about 10.0, alternatively from about 0.1 to about 8, or alternatively from about 0.5 to about 5; and wherein x2 balances the oxidation states. As will be appreciated by one of the skill in the art, and with the help of this disclosure, each of the La and Ce, and an optional redox agent can have multiple oxidation states within the OCM catalyst composition, and as such x2 can have any suitable value that allows for the oxygen anions to balance all the cations. Without wishing to be limited by theory, the different metals (La, Ce, optional redox agent(s)) present in the OCM catalyst composition as disclosed herein display synergetic effects in terms of conversion and selectivity. Further, and without wishing to be limited by theory, different ion radii and valences of the multiple metals (La, Ce, optional redox agent(s)) present in the OCM catalyst composition as disclosed herein can generate formation of uncompensated oxygen vacancies, which can lead to further improvement of catalyst performance, for example in terms of conversion, selectivity, stability, etc.

In an aspect, the OCM catalyst composition characterized by the general formula Laₐ₂Ce_{b2}Oₓ₂ and/or Laₐ₂Ce_{b2}D_{d2}Oₓ₂ as disclosed herein comprises one or more oxides of La, one or more oxides of Ce, optionally one or more oxides of redox agent (D), or a combination thereof. In such aspect, the one or more oxides can be present in the OCM catalyst composition in an amount of from about 0.01 wt. % to about 100.0 wt. %, alternatively from about 0.1 wt. % to about 99.0 wt. %, alternatively from about 1.0 wt. % to about 95.0 wt. %, alternatively from about 10.0 wt. % to about 90.0 wt. %, or alternatively from about 30.0 wt. % to about 70.0 wt. %, based on the total weight of the OCM catalyst composition. As will be appreciated by one of skill in the art, and with the help of this disclosure, and as previously described herein, it is possible that the OCM catalyst composition characterized by the general formula Laₐ₂Ce_{b2}Oₓ₂ and/or Laₐ₂Ce_{b2}D_{d2}Oₓ₂ as disclosed herein will comprise some hydroxides and/or some carbonates.

The one or more oxides in the OCM catalyst composition characterized by the general formula Laₐ₂Ce_{b2}Oₓ₂ and/or Laₐ₂Ce_{b2}D_{d2}Oₓ₂ as disclosed herein can comprise a single metal oxide, mixtures of single metal oxides, a mixed metal oxide, mixtures of mixed metal oxides, mixtures of single metal oxides and mixed metal oxides, or combinations thereof. The one or more oxides in the OCM catalyst composition characterized by the general formula Laₐ₂Ce_{b2}Oₓ₂ and/or Laₐ₂Ce_{b2}D_{d2}Oₓ₂ as disclosed herein can include, without limitation, La₂O₃, CeO₂, Ce₂O₃, Pr₂O₃, PrO₂, WO₃, MnO₂, W₂O₃, and the like, or combinations thereof.

In some aspects, the OCM catalyst composition characterized by the general formula Laₐ₂Ce_{b2}Oₓ₂ and/or Laₐ₂Ce_{b2}D_{d2}Oₓ₂ as disclosed herein can comprise a support, wherein at least a portion of the OCM catalyst composition contacts, coats, is embedded in, is supported by, and/or is distributed throughout at least a portion of the support.

In other aspects, the OCM catalyst composition characterized by the general formula Laₐ₂Ce_{b2}Oₓ₂ and/or Laₐ₂Ce_{b2}D_{d2}Oₓ₂ as disclosed herein is a unsupported OCM catalyst composition.

In an aspect, the OCM catalyst composition as disclosed herein can be a supported OCM catalyst composition comprising a silica (SiO₂) support, wherein the supported OCM catalyst composition is characterized by the general formula Mn—Na₂WO₄/SiO₂; wherein the supported OCM catalyst composition optionally comprises a metal oxide characterized by the general formula MOₓ₃; wherein M is a metal with redox properties; and wherein x3 balances the oxidation states.

In some aspects, the supported OCM catalyst composition as disclosed herein can be characterized by the general formula Mn—Na₂WO₄/SiO₂.

In other aspects, the supported OCM catalyst composition as disclosed herein can be characterized by the general formula (MOₓ₃)—Mn—Na₂WO₄/SiO₂; wherein M is a metal with redox properties (e.g., a redox metal M); and wherein x3 balances the oxidation states.

In yet other aspects, the supported OCM catalyst composition as disclosed herein can comprise a first catalyst component characterized by the general formula Mn—Na₂WO₄/SiO₂, and a second catalyst component characterized by the general formula (MOₓ₃)—Mn—Na₂WO₄/SiO₂. In such aspects, the supported OCM catalyst composition as disclosed herein can be regarded as a composite comprising the first catalyst component characterized by the general formula Mn—Na₂WO₄/SiO₂ and the second catalyst component characterized by the general formula (MOₓ₃)—Mn—Na₂WO₄/SiO₂, wherein the first catalyst component and the second catalyst component can be interspersed. In some aspects, the supported OCM catalyst composition can comprise a continuous first catalyst component characterized by the general formula Mn—Na₂WO₄/SiO₂ having a discontinuous second catalyst component characterized by the general formula (MOₓ₃)—Mn—Na₂WO₄/SiO₂ dispersed therein. In other aspects, the supported OCM catalyst composition can comprise a continuous second catalyst component characterized by the general formula (MOₓ₃)—Mn—Na₂WO₄/SiO₂ having a discontinuous first catalyst component characterized by the general formula Mn—Na₂WO₄/SiO₂ dispersed therein. In yet other aspects, the supported OCM catalyst composition can comprise both a continuous first catalyst component characterized by the general formula Mn—Na₂WO₄/SiO₂ and a continuous second catalyst component characterized by the general formula (MOₓ₃)—Mn—Na₂WO₄/SiO₂, wherein the first catalyst component and the second catalyst component contact each other. In still yet other aspects, the supported OCM catalyst composition can comprise regions of first catalyst component characterized by the general formula Mn—Na₂WO₄/SiO₂ and regions of second catalyst component characterized by the general formula (MOₓ₃)—Mn—Na₂WO₄/SiO₂, wherein at least a portion the regions of the first catalyst component contact at least a portion of the regions of the second catalyst component. As will be appreciated by one of skill in the art, and with the help of this disclosure, the amounts of each first catalyst component characterized by the general formula Mn—Na₂WO₄/SiO₂ and second catalyst component characterized by the general formula (MOₓ₃)—Mn—Na₂WO₄/SiO₂ present in the supported OCM catalyst composition contribute to the distribution of the first catalyst component and the second catalyst component within the supported OCM catalyst composition.

In aspects where the supported OCM catalyst composition characterized by the general formula Mn—Na₂WO₄/SiO₂ further comprises a metal oxide characterized by the general formula MOₓ₃, and as will be appreciated by one of skill in the art, and with the help of this disclosure, M can have multiple oxidation states within the supported OCM catalyst composition, and as such x3 can have any suitable value that allows for the oxygen anions to balance all the M cations in (MOₓ₃). Without wishing to be limited by theory, different metals (Na, Mn, W, and optionally M) present in the supported OCM catalyst compositions as disclosed herein can display synergetic effects in terms of conversion and selectivity. Further, and without wishing to be limited by theory, different ion radii and valences of the multiple metals (Na, Mn, W, and optionally M) present in the supported OCM catalyst compositions as disclosed herein can generate formation of surface oxygen vacancies (e.g., uncompensated oxygen vacancies), which can lead to further improvement of catalyst performance, for example in terms of conversion, selectivity, stability, etc.

When a chlorine intermediate precursor as disclosed herein is present in a reactant mixture that contacts a supported OCM catalyst composition characterized by the general formula Mn—Na₂WO₄/SiO₂ and/or (MOₓ₃)—Mn—Na₂WO₄/SiO₂ as disclosed herein (e.g., while conducting an OCM reaction), the chlorine intermediate precursor can interact with the catalyst (e.g., can form a chlorine intermediate), thereby leading to a stable catalyst performance over time. For example, and as would be appreciated by one of skill in the art, and with the help of this disclosure, conventional Mn—Na₂WO₄/SiO₂ catalysts suffer from poor stability over time owing to Na leaching out of the catalyst. However, in the presence of chlorine intermediate precursors in the reactant mixture as disclosed herein, the supported OCM catalyst composition comprising Mn—Na₂WO₄/SiO₂ (with or without the MOₓ₃ component) as disclosed herein displays an enhanced stability over time. Without wishing to be limited by theory, the Na in the supported OCM catalyst composition comprising Mn—Na₂WO₄/SiO₂ (with or without the MOₓ₃ component) as disclosed herein can react with the chlorine in the chlorine intermediate precursors and form a chlorine intermediate, such as sodium chloride, that can prevent Na leaching out of the supported OCM catalyst composition. Further, and without wishing to be limited by theory, the Mn in the supported OCM catalyst composition comprising Mn—Na₂WO₄/SiO₂ (with or without the MOₓ₃ component) as disclosed herein can react with the chlorine in the chlorine intermediate precursors and form a chlorine intermediate, such as manganese chloride. Furthermore, and without wishing to be limited by theory, while the formation of chlorine intermediates (e.g., sodium chloride, manganese chloride, etc.) can prevent leaching out of the active metals (e.g., Na, Mn, etc.) from the supported OCM catalyst composition comprising Mn—Na₂WO₄/SiO₂ (with or without the MOₓ₃ component), such chlorine intermediates can further prevent the redistribution and/or agglomeration of active catalyst phases (e.g., sodium, sodium tungstenate, manganese oxide, tungsten oxide, etc.) within supported OCM catalyst composition comprising Mn—Na₂WO₄/SiO₂ (with or without the MOₓ₃ component). As will be appreciated by one of skill in the art, and with the help of this disclosure, and without wishing to be limited by theory, conventional catalysts comprising Mn—Na₂WO₄/SiO₂ can have a tendency to display an agglomeration and redistribution of active phases owing to the formation of silicate compounds, such as manganese silicate and/or sodium silicate, which reduces catalyst performance in terms of activity and stability, by deactivating the catalyst. Without wishing to be limited by theory, the formation of chlorine intermediates (e.g., sodium chloride, manganese chloride, etc.) can prevent the redistribution and/or agglomeration of active catalyst phases within supported OCM catalyst composition comprising Mn—Na₂WO₄/SiO₂ (with or without the MOₓ₃ component) by reducing or eliminating the formation of silicate compounds, such as manganese silicate and/or sodium silicate.

As will be appreciated by one of skill in the art, and with the help of this disclosure, and without wishing to be limited by theory, Mn—Na₂WO₄/SiO₂ can display an enhanced activity towards CH₄ activation to form methyl radicals, for example according to reaction (1); however, if Mn—Na₂WO₄/SiO₂ were to be used in the absence of the chlorine intermediate precursor in the reactant mixture, the Mn—Na₂WO₄/SiO₂ could lead to an increased rate of reaction (1), which could further lead to an increased amount of hydroxyl groups [OH], on the surface of the catalyst, thus reducing catalyst activity, and potentially resulting in catalyst deactivation. Further, in aspects where the supported OCM catalyst composition as disclosed herein comprises MOₓ₃, and without wishing to be limited by theory, the MOₓ₃ portion of the supported OCM catalyst composition as disclosed herein can additionally enhance the re-oxidation step of the OCM reaction according to reaction (5), owing to the redox properties of M and/or MOₓ₃; and consequently the supported OCM catalyst composition as disclosed herein ((MOₓ₃)—Mn—Na₂WO₄/SiO₂) can perform the function of re-activating the reduced catalyst sites, thereby enhancing the activity and stability of the catalyst.

In an aspect, the supported OCM catalyst composition characterized by the general formula Mn—Na₂WO₄/SiO₂ and/or (MOₓ₃)—Mn—Na₂WO₄/SiO₂ as disclosed herein can comprise a Na—Mn—W component (i.e., Mn—Na₂WO₄), and optionally a redox metal oxide component (i.e., (MOₓ₃)); wherein the Na—Mn—W component, and optionally the redox metal oxide component are supported on silica (SiO₂). As will be appreciated by one of skill in the art, and with the help of this disclosure, and without wishing to be limited by theory, the redox metal oxide component and the Na—Mn—W component have different physical and chemical properties, owing to different chemical compositions, and as such can provide for optimum catalytic properties in different OCM reaction steps. Further, as will be appreciated by one of skill in the art, and with the help of this disclosure, the Na—Mn—W component can be used in the absence of the redox metal oxide component (MOₓ₃), and the supported OCM catalyst composition as disclosed herein can comprise the Na—Mn—W component supported on silica without the redox metal oxide component (MOₓ₃) (i.e., supported OCM catalyst composition characterized by the general formula Mn—Na₂WO₄/SiO₂). Furthermore, and as will be appreciated by one of skill in the art, and with the help of this disclosure, the Na—Mn—W component can be used in the presence of the redox metal oxide component (MOₓ₃), and the supported OCM catalyst composition as disclosed herein can comprise the Na—Mn—W component and the redox metal oxide component (MOₓ₃) supported on silica (i.e., supported OCM catalyst composition characterized by the general formula (MOₓ₃)—Mn—Na₂WO₄/SiO₂).

In aspects where the supported OCM catalyst composition characterized by the general formula (MOₓ₃)—Mn—Na₂WO₄/SiO₂ as disclosed herein comprises both the Na—Mn—W component and the redox metal oxide component, the supported OCM catalyst composition as disclosed herein can be regarded as a composite comprising the redox metal oxide component and the Na—Mn—W component, wherein the redox metal oxide component and the Na—Mn—W component can be interspersed. In some aspects, the supported OCM catalyst composition can comprise a continuous redox metal oxide component having a discontinuous Na—Mn—W component dispersed therein. In other aspects, the supported OCM catalyst composition can comprise a continuous Na—Mn—W component having a discontinuous redox metal oxide component dispersed therein. In yet other aspects, the supported OCM catalyst composition can comprise both a continuous redox metal oxide component and a continuous Na—Mn—W component, wherein the redox metal oxide component and the Na—Mn—W component contact each other. In still yet other aspects, the supported OCM catalyst composition can comprise regions of redox metal oxide component and regions of Na—Mn—W component, wherein at least a portion the regions of the redox metal oxide component contact at least a portion of the regions of the Na—Mn—W component. As will be appreciated by one of skill in the art, and with the help of this disclosure, the amounts of each redox metal oxide component and Na—Mn—W component present in the supported OCM catalyst composition contribute to the distribution of the redox metal oxide component and the Na—Mn—W component within the supported OCM catalyst composition. In aspects where the supported OCM catalyst composition as disclosed herein comprises both the Na—Mn—W component and the redox metal oxide component (i.e., supported OCM catalyst composition characterized by the general formula (MOₓ₃)—Mn—Na₂WO₄/SiO₂), as will be appreciated by one of skill in the art, and with the help of this disclosure, and without wishing to be limited by theory, the Na—Mn—W component and the redox metal oxide component are not chemically bonded to each other via covalent bonds, although physical interactions can exists between Na—Mn—W component and the redox metal oxide component, such as electrostatic interactions, van der Waals interactions, dipole-dipole interactions, ion-dipole interactions, etc.

In aspects where the supported OCM catalyst composition characterized by the general formula (MOₓ₃)—Mn—Na₂WO₄/SiO₂ as disclosed herein comprises both the Na—Mn—W component and the redox metal oxide component, the supported OCM catalyst composition as disclosed herein can be characterized by a weight ratio of MOₓ₃ to Mn—Na₂WO₄/SiO₂ of from about 0.01:1 to about 0.2:1, alternatively from about 0.02:1 to about 0.175:1, or alternatively from about 0.03:1 to about 0.15:1.

In an aspect, the Na—Mn—W component can comprise Mn—Na₂WO₄, Na/Mn/O, Na₂WO₄, Mn₂O₃—Na₂WO₄, Mn₃O₄—Na₂WO₄, MnWO₄—Na₂WO₄, MnWO₄—Na₂WO₄, Mn—WO₄, and the like, or combinations thereof. In an aspect, the Na—Mn—W component can comprise Mn—Na₂WO₄. In an aspect, the Na—Mn—W component can comprise an element with redox properties, such as manganese (Mn) and/or tungsten (W). As will be appreciated by one of skill in the art, and with the help of this disclosure, the redox metal M is a redox agent. Further, and as will be appreciated by one of skill in the art, and with the help of this disclosure, Mn and/or W can also be redox agents. Furthermore, and as will be appreciated by one of skill in the art, and with the help of this disclosure, although each of the redox metal oxide component and the Na—Mn—W component contain metals that are redox agents; all metals of the redox metal oxide component are redox agents; while some metals of the Na—Mn—W component are redox agents (e.g., Mn and/or W) and other metals of the Na—Mn—W component are not redox agents (e.g., Na).

In an aspect, the supported OCM catalyst composition characterized by the general formula Mn—Na₂WO₄/SiO₂ and/or (MOₓ₃)—Mn—Na₂WO₄/SiO₂ as disclosed herein can comprise manganese (Mn) in an amount of from about 0.1 wt. % to about 10 wt. %, alternatively from about 0.5 wt. % to about 9 wt. %, or alternatively from about 3 wt. % to about 8 wt. %, based on the total weight of the supported OCM catalyst composition.

In an aspect, the supported OCM catalyst composition characterized by the general formula Mn—Na₂WO₄/SiO₂ and/or (MOₓ₃)—Mn—Na₂WO₄/SiO₂ as disclosed herein can comprise Na₂WO₄ in an amount of from about 0.1 wt. % to about 15 wt. %, alternatively from about 0.5 wt. % to about 12.5 wt. %, or alternatively from about 1 wt. % to about 10 wt. %, based on the total weight of the supported OCM catalyst composition.

In an aspect, the redox metal M (e.g., metal M with redox properties, redox agent M) can be selected from the group consisting of tin (Sn), antimony (Sb), bismuth (Bi), iron (Fe), chromium (Cr), molybdenum (Mo), tungsten (W), vanadium (V), tantalum (Ta), niobium (Nb), gallium (Ga), rhenium (Re), lead (Pb), cobalt (Co), nickel (Ni), copper (Cu), and combinations thereof. A redox metal generally refers to a chemical metal species that possesses the ability to undergo both an oxidation reaction and a reduction reaction, and such ability usually resides in the chemical metal species having more than one stable oxidation state other than the oxidation state of zero (0). As will be appreciated by one of skill in the art, and with the help of this disclosure, tungsten (W) when present in the redox metal oxide component is not in the form of Na₂WO₄; while tungsten (W) when present in the Na—Mn—W component is in the form of Na₂WO₄.

In some aspects, the redox metal M comprises Sn. In other aspects, the redox metal M comprises Sb. In yet other aspects, the redox metal M comprises Pb. As will be appreciated by one of skill in the art, and with the help of this disclosure, in some aspects, the redox metal M can comprise a single redox metal, such as Sn or Sb.

In still yet other aspects, the redox metal M comprises Mo. In still yet other aspects, the redox metal M comprises V. As will be appreciated by one of skill in the art, and with the help of this disclosure, in some aspects, the redox metal M can comprise two or more redox metals. For example, the redox metal M can comprise Mo, Bi and Fe. As another example, the redox metal M can comprise V, Bi and Fe.

In an aspect, the redox metal M excludes a rare earth element.

In an aspect, the redox metal M can be basic (e.g., can exhibit some degree of basicity; can have affinity for hydrogen; can exhibit some degree of affinity for hydrogen). Nonlimiting examples of redox metals M that can be considered basic for purposes of the disclosure herein include tantalum (Ta), rhenium (Re), lead (Pb), and combinations thereof. As will be appreciated by one of skill in the art, and with the help of this disclosure, and without wishing to be limited by theory, the OCM reaction is a multi-step reaction, wherein each step of the OCM reaction could benefit from specific OCM catalytic properties.

In an aspect, the supported OCM catalyst composition as disclosed herein can comprise one or more oxides of M (e.g., MOₓ₃). In some aspects, the redox metal oxide component of the supported OCM catalyst composition can comprise, consist of, or consist essentially of the one or more oxides of M (e.g., redox metal M oxides (MOₓ₃)).

In an aspect, the one or more oxides of M (e.g., MOₓ₃) can be present in the redox metal oxide component of the supported OCM catalyst composition in an amount of from about 0.01 wt. % to about 100.0 wt. %, alternatively from about 0.1 wt. % to about 99.0 wt. %, alternatively from about 1.0 wt. % to about 95.0 wt. %, alternatively from about 10.0 wt. % to about 90.0 wt. %, or alternatively from about 30.0 wt. % to about 70.0 wt. %, based on the total weight of redox metal oxide component of the supported OCM catalyst composition. As will be appreciated by one of skill in the art, and with the help of this disclosure, the redox metal oxide component of the supported OCM catalyst composition may comprise some hydroxides and/or some carbonates.

In an aspect, the supported OCM catalyst composition characterized by the general formula (MOₓ₃)—Mn—Na₂WO₄/SiO₂ as disclosed herein comprises the metal M in an amount of from about 0.1 wt. % to about 10 wt. %, alternatively from about 0.5 wt. % to about 7.5 wt. %, or alternatively from about 1 wt. % to about 5 wt. %, based on the total weight of the supported OCM catalyst composition.

In an aspect, the one or more oxides of M (e.g., MOₓ₃) can comprise a single metal oxide, mixtures of single metal oxides, a mixed metal oxide, mixtures of mixed metal oxides, mixtures of single metal oxides and mixed metal oxides, or combinations thereof.

The single metal oxide comprises one redox metal M (e.g., a single redox metal M). Nonlimiting examples of single metal oxides suitable for use in the supported OCM catalyst composition characterized by the general formula (MOₓ₃)—Mn—Na₂WO₄/SiO₂ as disclosed herein include Sb₂O₃, SnO₂, Sb₂O₅, SnO, FeO, Fe₂O₃, Fe₃O₄, Mo₂O₃, Mo₂O₅, MoO₃, W₂O₃, W₂O₅, WO₃, Cr₂O₃, Cr₂O₅, NiO, Ni₂O₃, CoO, Co₂O₃, Co₃O₄, and the like, or combinations thereof.

Mixtures of single metal oxides suitable for use in the supported OCM catalyst composition characterized by the general formula (MOₓ₃)—Mn—Na₂WO₄/SiO₂ as disclosed herein can comprise two or more different single metal oxides, wherein each single metal oxide can be selected from the group consisting of Sb₂O₃, SnO₂, Sb₂O₅, SnO, FeO, Fe₂O₃, Fe₃O₄, Mo₂O₃, Mo₂O₅, MoO₃, W₂O₃, W₂O₅, WO₃, Cr₂O₃, Cr₂O₅, NiO, Ni₂O₃, CoO, Co₂O₃, and Co₃O₄. Nonlimiting examples of mixtures of single metal oxides suitable for use in the suitable for use in the supported OCM catalyst composition characterized by the general formula (MOₓ₃)—Mn—Na₂WO₄/SiO₂ as disclosed herein include Sb₂O₃—SnO₂, Sb₂O₅—SnO, Sb₂O₃—W₂O₃, SnO₂—WO₃, Sb₂O₃—SnO₂—WO₃, and the like, or combinations thereof.

The mixed metal oxide comprises two or more different redox metals M. Nonlimiting examples of mixed metal oxides suitable for use in the supported OCM catalyst composition characterized by the general formula (MOₓ₃)—Mn—Na₂WO₄/SiO₂ as disclosed herein include FeMoO₄; CoMoO₄; NiMoO₄; FeWO₄; CoWO₄; NiWO₄; PbMoO₄; PbWO₄; CuMoO₄; CuWO₄; and the like; or combinations thereof.

Mixtures of mixed metal oxides suitable for use in the supported OCM catalyst composition characterized by the general formula (MOₓ₃)—Mn—Na₂WO₄/SiO₂ as disclosed herein can comprise two or more different mixed metal oxides, wherein each mixed metal oxide can be selected from the group consisting of FeMoO₄; CoMoO₄; NiMoO₄; FeWO₄; CoWO₄; NiWO₄; PbMoO₄; PbWO₄; CuMoO₄; and CuWO₄.

The redox metal oxide component (e.g., MOₓ₃) of the supported OCM catalyst composition characterized by the general formula (MOₓ₃)—Mn—Na₂WO₄/SiO₂ as disclosed herein can have any suitable desired shape and/or size specifications, for example as required by a specific application. In some aspects, the MOₓ₃ can comprise nanostructures, wherein a nanostructure is defined as a three-dimensional object characterized by at least one external dimension of less than about 1,000 nm. As will be appreciated by one of skill in the art, and with the help of this disclosure, and without wishing to be limited by theory, three-dimensional objects are characterized by three external dimensions. For example, any three-dimensional object can be placed in a three-dimensional Cartesian coordinate system (i.e., a Cartesian coordinate system for a three-dimensional space) having axes x, y, and z, wherein the three-dimensional object is characterized by a first external dimension along x, a second external dimension along y, and a third external dimension along z. In some aspects, the redox metal oxide component (e.g., MOₓ₃) of the supported OCM catalyst can comprise nanoparticles, nanofibers, nanoplates, or combinations thereof; wherein nanoparticles, nanofibers, and nanoplates are three-dimensional objects defined in accordance with ISO/TS 80004-2:2015.

In an aspect, the supported OCM catalyst composition characterized by the general formula Mn—Na₂WO₄/SiO₂ and/or (MOₓ₃)—Mn—Na₂WO₄/SiO₂ as disclosed herein comprises a silica (SiO₂) support, wherein at least a portion of the supported OCM catalyst composition (e.g., the Na—Mn—W component, and optionally the redox metal oxide component) contacts, coats, is embedded in, is supported by, and/or is distributed throughout at least a portion of the support. As will be appreciated by one of skill in the art, and with the help of this disclosure, the support (i.e., SiO₂) is catalytically inactive or non-selective (e.g., the support cannot catalyze an OCM reaction or cannot give high selectivity). Further, as will be appreciated by one of skill in the art, and with the help of this disclosure, the silica support can be purchased or can be prepared by using any suitable methodology, such as for example precipitation/co-precipitation, sol-gel techniques, templates/surface derivatized metal oxides synthesis, solid-state synthesis of metal oxides, microemulsion techniques, solvothermal techniques, sonochemical techniques, combustion synthesis, etc. In some aspects, the silica support can be a porous support.

In an aspect, the supported OCM catalyst composition characterized by the general formula Mn—Na₂WO₄/SiO₂ and/or (MOₓ₃)—Mn—Na₂WO₄/SiO₂ as disclosed herein can comprise SiO₂ in an amount of from about 5 wt. % to about 95 wt. %, alternatively from about 25 wt. % to about 75 wt. %, or alternatively from about 35 wt. % to about 65 wt. %, based on the total weight of the supported OCM catalyst composition. As will be appreciated by one of skill in the art, and with the help of this disclosure, the amount of catalytically active material composition (e.g., the Na—Mn—W component, and optionally the redox metal oxide component) on the support, and consequently the amount of support in the catalyst composition, depends on the catalytic activity of the catalytically active material.

In some aspects, the supported OCM catalyst composition characterized by the general formula (MOₓ₃)—Mn—Na₂WO₄/SiO₂ as disclosed herein has the general formula (SbOₓ₃)—Mn—Na₂WO₄/SiO₂.

In an aspect, a process for producing C₂₊ hydrocarbons (e.g., C₂₊ olefins, C₂₊ alkanes, etc.) as disclosed herein can comprise the steps of (a) introducing a reactant mixture to a reactor comprising an OCM catalyst composition, wherein the reactant mixture comprises CH₄, O₂, and a chlorine intermediate precursor, wherein the chlorine intermediate precursor is introduced into the reactor in an amount of from about from about 1 ppm to about 100 ppm, based on the total volume of the reactant mixture, and wherein the OCM catalyst composition is characterized by the general formula Srₐ₁La_{b1}E_{c1}D_{d1}Oₓ₁; wherein E is a rare earth element (e.g., second rare earth element); wherein D is another rare earth element; wherein D and E are not lanthanum and are not the same; wherein a1 is 1.0; wherein b1 is from about 0.3 to about 10.0, alternatively from about 0.6 to about 1.2, or alternatively from about 0.8 to about 1.0; wherein c1 is from about 0.01 to about 10.0, alternatively from about 0.4 to about 1, or alternatively from about 0.6 to about 0.8; wherein d1 is from about 0.01 to about 10.0, alternatively from about 0.05 to about 0.15, or alternatively from about 0.09 to about 0.12; and wherein x1 balances the oxidation states; (b) allowing at least a portion of the reactant mixture to contact at least a portion of the OCM catalyst composition and react via an OCM reaction at an OCM reaction temperature to form a product mixture comprising C₂₊ hydrocarbons (e.g., C₂₊ olefins, C₂₊ alkanes, etc.); and (c) recovering at least a portion of the C₂₊ hydrocarbons (e.g., C₂₊ olefins, C₂₊ alkanes, etc.) from the product mixture. In some aspects, b1 is 9, c1 is 1, and d1 is 7. The rare earth element E and the another rare earth element D can be selected from the group consisting of scandium (Sc), cerium (Ce), praseodymium (Pr), neodymium (Nd), promethium (Pm), samarium (Sm), europium (Eu), gadolinium (Gd), yttrium (Y), terbium (Tb), dysprosium (Dy), holmium (Ho), erbium (Er), thulium (Tm), ytterbium (Yb), lutetium (Lu), and combinations thereof. In some aspects, the OCM catalyst composition characterized by the general formula Srₐ₁La_{b1}E_{c1}D_{d1}Oₓ₁ further comprises a redox agent selected from the group consisting of manganese (Mn), tungsten (W), bismuth (Bi), antimony (Sb), tin (Sn), cerium (Ce), praseodymium (Pr), and combinations thereof. In such aspects, the redox agent can comprise manganese (Mn), tin (Sn), bismuth (Bi), cerium (Ce), or combinations thereof. In an aspect, the redox agent can be present in the OCM catalyst composition characterized by the general formula Srₐ₁La_{b1}E_{c1}D_{d1}Oₓ₁ in an amount of from about 1 wt. % to about 25 wt. %, based on the total weight of the OCM catalyst composition. In some aspects, OCM catalyst composition characterized by the general formula Srₐ₁La_{b1}E_{c1}D_{d1}Oₓ₁ comprises one or more oxides; wherein the one or more oxides can include one or more oxides of strontium; one or more oxides of lanthanum; one or more oxides of D; one or more oxides of E; or combinations thereof. In some aspects, the OCM catalyst composition can have the general formula Srₐ₁La_{b1}Yb_{c1}D_{d1}Oₓ₁, wherein a1 is 1.0; wherein b1 is from about 0.3 to about 10.0, alternatively from about 0.6 to about 1.2, or alternatively from about 0.8 to about 1.0; wherein c1 is from about 0.01 to about 10.0, alternatively from about 0.4 to about 1, or alternatively from about 0.6 to about 0.8; wherein d1 is from about 0.01 to about 10.0, alternatively from about 0.05 to about 0.15, or alternatively from about 0.09 to about 0.12; and wherein x1 balances the oxidation states.

In an aspect, a process for producing C₂₊ hydrocarbons (e.g., C₂₊ olefins, C₂₊ alkanes, etc.) as disclosed herein can comprise the steps of (a) introducing a reactant mixture comprising CH₄, O₂, and a chlorine intermediate precursor to a reactor comprising an OCM catalyst composition; wherein the OCM catalyst composition is a lanthanum-cerium oxide catalyst characterized by the general formula Laₐ₂Ce_{b2}Oₓ₂; wherein a2 is 1.0; wherein b2 is from about 0.05 to about 1.0, alternatively from about 0.06 to about 0.08, or alternatively from about 0.07 to about 0.1; and wherein x2 balances the oxidation states; (b) allowing at least a portion of the reactant mixture to contact at least a portion of the OCM catalyst composition and react via an OCM reaction at an OCM reaction temperature to form a product mixture comprising C₂₊ hydrocarbons (e.g., C₂₊ olefins, C₂₊ alkanes, etc.); and (c) recovering at least a portion of the C₂₊ hydrocarbons (e.g., C₂₊ olefins, C₂₊ alkanes, etc.) from the product mixture. In some aspects, the OCM catalyst composition characterized by the general formula Laₐ₂Ce_{b2}Oₓ₂ can further comprise a redox agent selected from the group consisting of manganese (Mn), tungsten (W), bismuth (Bi), antimony (Sb), tin (Sn), cerium (Ce), praseodymium (Pr), and combinations thereof. In some aspects, the redox agent comprises manganese (Mn), tin (Sn), bismuth (Bi), cerium (Ce), or combinations thereof. In some aspects, the redox agent is present in the OCM catalyst composition characterized by the general formula Laₐ₂Ce_{b2}Oₓ₂ in an amount of from about 1 wt. % to about 25 wt. %, based on the total weight of the OCM catalyst composition. In some aspects, the OCM catalyst composition characterized by the general formula Laₐ₂Ce_{b2}Oₓ₂ comprises one or more oxides; wherein the one or more oxides can include one or more oxides of lanthanum; one or more oxides of cerium; or combinations thereof.

In an aspect, a process for producing C₂₊ hydrocarbons (e.g., C₂₊ olefins, C₂₊ alkanes, etc.) as disclosed herein can comprise the steps of (a) introducing a reactant mixture to an OCM reactor comprising a supported OCM catalyst composition; wherein the reactant mixture comprises CH₄, O₂, and a chlorine intermediate precursor; wherein the chlorine intermediate precursor is present in the reactant mixture in an amount of from about 4 ppm to about 25 ppm, based on the total volume of the reactant mixture; wherein the supported OCM catalyst composition is characterized by the general formula (SbOₓ₃)—Mn—Na₂WO₄/SiO₂; wherein x3 balances the oxidation states; wherein the OCM reactor is characterized by a reactor effluent temperature of from about 800° C. to about 875° C.; wherein the OCM reactor is characterized by a residence time of from about 500 milliseconds (ms) to about 1.5 seconds (s); and wherein the reactant mixture is characterized by a CH₄/O₂ molar ratio of from about 5:1 to about 8:1; (b) allowing at least a portion of the reactant mixture to contact at least a portion of the supported OCM catalyst composition and react via an OCM reaction to form a product mixture comprising unreacted methane and C₂₊ hydrocarbons (e.g., C₂₊ olefins, C₂₊ alkanes, etc.); (c) recovering at least a portion of the product mixture from the OCM reactor; and (d) recovering at least a portion of the C₂₊ hydrocarbons (e.g., C₂₊ olefins, C₂₊ alkanes, etc.) from the product mixture. As will be appreciated by one of the skill in the art, and with the help of this disclosure, Sb can have multiple oxidation states within the supported OCM catalyst composition, and as such x3 can have any suitable value that allows for the oxygen anions to balance all the cations in the redox metal oxide component (e.g., SbOₓ₃) of the supported OCM catalyst composition. In such aspect, SbOₓ₃ can comprise Sb₂O₃.

In an aspect, a process for producing C₂₊ hydrocarbons (e.g., C₂₊ olefins, C₂₊ alkanes, etc.) as disclosed herein can comprise the steps of (a) introducing a reactant mixture to an OCM reactor comprising a supported OCM catalyst composition; wherein the reactant mixture comprises CH₄, O₂, and a chlorine intermediate precursor; wherein the chlorine intermediate precursor is present in the reactant mixture in an amount of from about 4 ppm to about 25 ppm; wherein the supported OCM catalyst composition is characterized by the general formula (SnOₓ₃)—Mn—Na₂WO₄/SiO₂; wherein x3 balances the oxidation states; wherein the OCM reactor is characterized by a reactor effluent temperature of from about 800° C. to about 875° C.; wherein the OCM reactor is characterized by a residence time of from about 500 milliseconds (ms) to about 1.5 seconds (s); and wherein the reactant mixture is characterized by a CH₄/O₂ molar ratio of from about 5:1 to about 8:1; (b) allowing at least a portion of the reactant mixture to contact at least a portion of the supported OCM catalyst composition and react via an OCM reaction to form a product mixture comprising unreacted methane and C₂₊ hydrocarbons (e.g., C₂₊ olefins, C₂₊ alkanes, etc.)s; (c) recovering at least a portion of the product mixture from the OCM reactor; and (d) recovering at least a portion of the C₂₊ hydrocarbons (e.g., C₂₊ olefins, C₂₊ alkanes, etc.) from the product mixture. As will be appreciated by one of the skill in the art, and with the help of this disclosure, Sn can have multiple oxidation states within the supported OCM catalyst composition, and as such x can have any suitable value that allows for the oxygen anions to balance all the cations in the redox metal oxide component (e.g., SnOₓ₃) of the supported OCM catalyst composition. In such aspect, SnOₓ₃ can comprise SnO₂.

In an aspect, a process for producing C₂₊ hydrocarbons (e.g., C₂₊ olefins, C₂₊ alkanes, etc.) as disclosed herein can comprise the steps of (a) introducing a reactant mixture to a reactor comprising an OCM catalyst composition, wherein the reactant mixture comprises CH₄, O₂, and CH₃Cl, wherein the CH₃Cl is introduced continuously into the reactor in an amount of from about from about 1 ppm to about 50 ppm, alternatively from about 5 ppm to about 25 ppm, or alternatively from about 10 ppm to about 20 ppm, based on the total volume of the reactant mixture, and wherein the OCM catalyst composition is characterized by the general formula Aₐ₁Z_{b1}E_{c1}D_{d1}Oₓ₁, wherein A is an alkaline earth metal; wherein Z is a first rare earth element; wherein E is a second rare earth element; wherein D is a redox agent or a third rare earth element; wherein the first rare earth element, the second rare earth element, and the third rare earth element, when present, are not the same; wherein a1 is 1.0; wherein b1 is from about 0.1 to about 10.0, alternatively from about 0.3 to about 10.0, alternatively from about 0.5 to about 8, or alternatively from about 1 to about 5; wherein c1 is from about 0 to about 10.0, alternatively from about 0.1 to about 10, alternatively from about 0.1 to about 8, or alternatively from about 0.5 to about 5; wherein d1 is from about 0 to about 10.0, alternatively from about 0.1 to about 8, or alternatively from about 0.5 to about 5; and wherein x1 balances the oxidation states; (b) allowing at least a portion of the reactant mixture to contact at least a portion of the OCM catalyst composition and react via an OCM reaction at an OCM reaction temperature to form a product mixture comprising C₂₊ hydrocarbons (e.g., C₂₊ olefins, C₂₊ alkanes, etc.); and (c) recovering at least a portion of the C₂₊ hydrocarbons (e.g., C₂₊ olefins, C₂₊ alkanes, etc.) from the product mixture. In such aspect, the process for producing C₂₊ hydrocarbons (e.g., C₂₊ olefins, C₂₊ alkanes, etc.) as disclosed herein can be carried out at a temperature of from about 750° C. to about 850° C., or alternatively from about 820° C. to about 825° C. In such aspect, the process for producing C₂₊ hydrocarbons (e.g., C₂₊ olefins, C₂₊ alkanes, etc.) as disclosed herein can be characterized by a residence time of less than about 10 ms, or alternatively less than about 5 ms. In such aspect, the process displays an increase in C₂₊ selectivity, wherein the increased C₂₊ selectivity can be maintained in a stable manner over a time period of equal to or greater than about 100 h. In such aspect, the OCM catalyst composition characterized by the general formula Aₐ₁Z_{b1}E_{c1}D_{d1}Oₓ₁, can be a mixed oxide catalyst comprising one or more oxides of Sr, one or more oxides of La, one or more oxides of Yb, one or more oxides of Nd, one or more oxides of Ce, one or more oxides of Sm, one or more oxides of Ba, or combinations thereof. In some aspects, the OCM catalyst composition characterized by the general formula Aₐ₁Z_{b1}E_{c1}D_{d1}Oₓ₁, can be a mixed oxide catalyst comprising one or more oxides of Sr, one or more oxides of La, one or more oxides of Yb, and one or more oxides of Nd.

In an aspect, a process for producing C₂₊ hydrocarbons (e.g., C₂₊ olefins, C₂₊ alkanes, etc.) as disclosed herein can advantageously mitigate the heat management in an OCM reactor by operating the reactor in an autothermal mode. As will be appreciated by one of skill in the art, and with the help of this disclosure, the OCM reaction is very exothermic reaction due to the formation of deep oxidation products (CO, CO₂) that are conventionally produced via OCM with a selectivity about 20-25%. The conventional adiabatic temperature rise in an adiabatically operated OCM reactor can typically be in the range of 600° C.-1,000° C., depending upon the CH₄/O₂ molar ratio in the feed. Additionally, in conventional OCM processes, the heat release rate in the OCM reactor at temperatures of about 900° C. can be two to three orders of magnitude higher than rate at which the heat can be removed in a cooled multitubular reactor, for example. In an autothermal operation, the reactor can be placed in a desired state by first “igniting” the catalyst at a suitable temperature and then changing both the feed composition and the feed temperature in a defined way so as to put the system in the desired state of autothermal operation. At steady-state, an autothermal process can use cold feed to absorb the heat of the reaction such that the catalyst bed would be maintained at a desired temperature, typically from about 700° C. to about 1,000° C., alternatively from about 750° C. to about 950° C., or alternatively from about 800° C. to about 900° C.; while the cold feed, typically at temperatures from about 25° C. to about 400° C., alternatively from about 100° C. to about 400° C., or alternatively from about 200° C. to about 400° C., can absorb the heat of the reaction to sustain the OCM reaction.

As will be appreciated by one of skill in the art, and without wishing to be limited by theory, the term “autothermal” can refer to the reaction being carried out by the heat of the reaction without application of external heating resources (e.g., in the absence of external heat input, in the absence of an external heat supply). The autothermal operation of the reactor as disclosed herein may occur when the catalyst can be ignited at a certain temperature, for example at about 450-650° C., which in turn produces heat in an OCM process. Subsequent to starting heat production by the OCM reaction, the external heating resources can be turned off gradually. When external heating is turned off, the temperature of the catalyst (e.g., temperature of the catalyst bed) may decrease; because at the beginning of the process, the reaction is carried out by a total heat which is the heat of the reaction+heat of furnace. When the power to the furnace is gradually decreased, in order to compensate for the other part of the heat (heat of furnace, which is being gradually turned off) in the total heat (reaction heat+external heat), it becomes necessary to produce additional heat via production of products that generate more heat, such as CO₂. In order to achieve this heat compensation via production of products that generate more heat (e.g., CO₂), the decrease in furnace power can be accompanied by gradually increasing the O₂/CH₄ ratio (i.e., decreasing the CH₄/O₂ ratio). Autothermal processes for OCM as disclosed herein have been observed experimentally, and the experimental procedure and results are described in more detail later herein in the Examples, e.g., Example 17.

In an aspect, a process for producing C₂₊ hydrocarbons (e.g., C₂₊ olefins, C₂₊ alkanes, etc.) under autothermal conditions as disclosed herein can advantageously provide for a desired good methane conversion and selectivity to C₂₊ hydrocarbons. The process for producing C₂₊ hydrocarbons (e.g., C₂₊ olefins, C₂₊ alkanes, etc.) under autothermal conditions as disclosed herein does not use any external heat exchanger to transfer heat from the reactor bed to the feed. Furthermore, an adiabatic autothermal operation as disclosed herein does not use any other external source of heat to keep the catalyst bed in an ignited state once the steady state has been reached.

In an aspect, a process for producing C₂₊ hydrocarbons (e.g., C₂₊ olefins, C₂₊ alkanes, etc.) under near-isothermal and/or autothermal conditions as disclosed herein can advantageously employ relatively low levels of a chlorine intermediate precursor (e.g., from about 1 ppm to about 100 ppm, based on the total volume of the reactant mixture) along with methane and oxygen in the feed to the OCM reactor to improve the C₂₊ selectivity under near-isothermal and/or autothermal operation. In such aspect, the reactor can comprise an OCM catalyst composition having the general formula Srₐ₁La_{b1}Oₓ₁/MgO; wherein a1 is 1.0; wherein b 1 is from about 0.1 to about 10.0; and wherein x1 balances the oxidation states. In such aspect, the OCM catalyst composition can be characterized by the general formula Sr_{1.0}La_{0.18}Mg_{0.13}Oₓ₁. In such aspect, the OCM catalyst composition having the general formula Srₐ₁La_{b1}Oₓ₁/MgO can lead to a C₂₊ selectivity of equal to or greater than about 60% in a temperature range of from about 700° C. to about 950° C.

In an aspect, a process for producing C₂₊ hydrocarbons (e.g., C₂₊ olefins, C₂₊ alkanes, etc.) under near-isothermal and/or autothermal conditions as disclosed herein can advantageously employ pre-treatment of OCM catalysts at relatively low room temperature (e.g., from about 25° C. to about 500° C., alternatively from about 25° C. to about 400° C., alternatively from about 25° C. to about 200° C., alternatively from about 25° C. to about 100° C., alternatively from about 25° C. to about 50° C., or alternatively about room temperature) with relatively low levels of a chlorine intermediate precursor (e.g., from about 1 ppm to about 100 ppm, based on the total volume of the pre-treating mixture) to improve the C₂₊ selectivity under near-isothermal and/or autothermal operation. In such aspect, the reactor can comprise an OCM catalyst composition having the general formula Srₐ₁La_{b1}Oₓ₁/MgO; wherein a1 is 1.0; wherein b 1 is from about 0.1 to about 10.0; and wherein x1 balances the oxidation states. In such aspect, the OCM catalyst composition can be characterized by the general formula Sr_{1.0}La_{0.18}Mg_{0.13}Oₓ₁. In such aspect, the OCM catalyst composition having the general formula Srₐ₁La_{b1}Oₓ₁/MgO can lead to a C₂₊ selectivity of equal to or greater than about 60% in a temperature range of from about 700° C. to about 950° C. In some aspects, the OCM catalyst compositions suitable for use in the process for producing C₂₊ hydrocarbons (e.g., C₂₊ olefins, C₂₊ alkanes, etc.) under near-isothermal and/or autothermal conditions as disclosed herein can display an initial C₂₊ selectivity (in the absence of a chlorine intermediate precursor and/or absent a pre-treatment with a chlorine intermediate precursor as disclosed herein) of equal to or greater than about 60%, or alternatively equal to or greater than about 70%, wherein the C₂₊ selectivity of such OCM catalyst composition is increased in the process for producing C₂₊ hydrocarbons (e.g., C₂₊ olefins, C₂₊ alkanes, etc.) under near-isothermal and/or autothermal conditions as disclosed herein.

In an aspect, a process for producing C₂₊ hydrocarbons (e.g., C₂₊ olefins, C₂₊ alkanes, etc.) under near-isothermal and/or autothermal conditions as disclosed herein can advantageously employ relatively low levels of a chlorine intermediate precursor (e.g., from about 1 ppm to about 100 ppm, based on the total volume of the reactant mixture) along with methane and oxygen in the feed to the OCM reactor to improve C₂₊ selectivity and/or decrease CO₂ formation (and consequently decrease production of heat). As such, maintaining a substantially constant catalyst bed temperature with addition of a chlorine intermediate precursor to the reactant mixture under autothermal condition entails selection of conditions, including but not limited to amount of dosing of organic chloride, CH₄/O₂ molar ratio, level of catalyst bed temperature which is necessary to keep bed ignited and prevent extinction of the catalyst bed in an autothermal operation; and the like, or combinations thereof.

In an aspect, the process of producing C₂₊ hydrocarbons (e.g., C₂₊ olefins, C₂₊ alkanes, etc.) via an OCM process as disclosed herein can advantageously display improvements in one or more process characteristics when compared to conventional methods of producing C₂₊ hydrocarbons (e.g., C₂₊ olefins, C₂₊ alkanes, etc.). Conventionally, OCM reactions to C₂₊ hydrocarbons proceed with the formation of deep oxidation products which leads to runway of reaction heat; and thus reduction of deep oxidation reactions at high temperatures is very important in OCM. The process for producing C₂₊ hydrocarbons (e.g., C₂₊ olefins, C₂₊ alkanes, etc.) as disclosed herein advantageously reduces the formation of deep oxidation products by addition of chlorine intermediate precursors, such as CH₃Cl, to the OCM feed.

In an aspect, the process of producing C₂₊ hydrocarbons (e.g., C₂₊ olefins, C₂₊ alkanes, etc.) via OCM as disclosed herein can advantageously display improvements in process performance in the presence of the chlorine intermediate precursors in the reactant mixture, such as increased C₂₊ selectivity, decreased selectivity to carbon dioxide (CO₂), increased catalyst activity, increased methane conversion, increased catalyst stability, increased catalyst life-time, decreased reaction temperature effective for achieving an O₂ conversion of equal to or greater than about 90%, and combinations thereof. The OCM process conducted in the presence of the chlorine intermediate precursors in the reactant mixture with the OCM catalyst compositions as disclosed herein display improved catalyst stability, wherein the chlorine intermediate precursor can advantageously prevent agglomeration of active phase of the catalyst and redistribution of phase composition during the relatively high temperatures of the OCM reaction (e.g., equal to or greater than about 700° C.). The process performance improvements are advantageously stable overextended time periods, for example equal to or greater than about 100 h. Additional advantages of the methods of producing C₂₊ hydrocarbons (e.g., C₂₊ olefins, C₂₊ alkanes, etc.) via an OCM process as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Na—Mn—W Catalysts (Mn—Na₂WO₄/SiO₂ and/or (MOₓ₃)—Mn—Na₂WO₄/SiO₂)

### Example 1

Oxidative coupling of methane (OCM) catalyst compositions were prepared as follows.

A supported catalyst characterized by the general formula Mn—Na₂WO₄/SiO₂ was prepared by using the following procedure. Silica gel (18.6 g, Davisil® Grade 646) was used after drying overnight. Mn(NO₃)₂.4H₂O (1.73 g) was dissolved in deionized water (18.5 mL), and then added dropwise onto the silica gel. The resulting manganese impregnated silica material was dried overnight. Na₂WO₄.4H₂O (1.22 g) was dissolved in deionized water (18.5 mL), and the solution obtained was added onto the dried manganese silica material above. The resulting material obtained was dried overnight at 125° C., and then calcined at 800° C. for 6 hours under airflow to obtain the Mn—Na₂WO₄/SiO₂ catalyst.

A supported catalyst characterized by the general formula (Sb₂O₃)—Mn—Na₂WO₄/SiO₂ was prepared by using the following method. 0.11 g of Sb₂O₃ (with particle size of 80-200 nm) was mixed with deionized water (6.0 mL) to form a slurry. The slurry was then added onto 3.3 g of calcined Mn—Na₂WO₄/SiO₂ catalyst prepared as described above. The resulting mixture was dried overnight at 125° C., and then calcined at 800° C. for 6 hours under airflow to obtain the (Sb₂O₃)—Mn—Na₂WO₄/SiO₂ catalyst ((3 wt. % Sb₂O₃)—Mn—Na₂WO₄/SiO₂ catalyst).

### Example 2

The performance of the supported OCM catalyst compositions prepared as described in Example 1 was investigated. Specifically, the performance of the Mn—Na₂WO₄/SiO₂ catalyst and the (Sb₂O₃)—Mn—Na₂WO₄/SiO₂ catalyst was investigated in an OCM reaction in the presence and in the absence of a chlorine intermediate precursor (CH₃Cl). OCM reactions were conducted by using catalysts prepared as described in Example 1 as follows.

Performance Test.

The catalysts obtained as described in Example 1 were performance tested in a 4.0 mm ID quartz tube reactor. The reactor was loaded with 100 mg catalyst. An OCM feed mixture of was fed to the reactor at a flow rate of 34.8 cc/min CH₄, 5.1 cc/min O₂, and 34.8 cc/min N₂. CH₄ feed contained 20 ppm CH₃Cl. Products obtained were analyzed by using online GC with TCD and FID detectors.

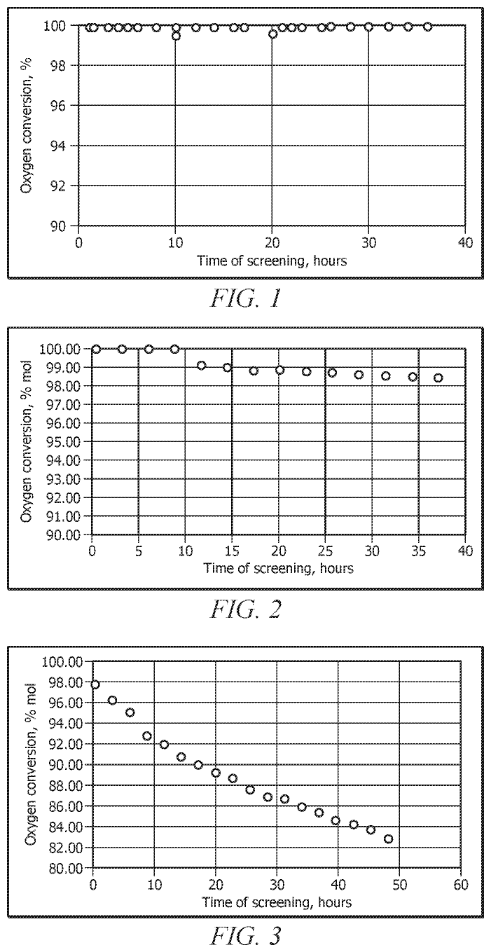

FIG. 1 displays the O₂ conversion for the (Sb₂O₃)—Mn—Na₂WO₄/SiO₂ catalyst in the presence of 20 ppm CH₃Cl in the feed. The (Sb₂O₃)—Mn—Na₂WO₄/SiO₂ catalyst displays about 100% O₂ conversion for the entire time on stream (TOS) with no apparent decline in catalyst activity.

FIG. 2 displays the O₂ conversion for the (Sb₂O₃)—Mn—Na₂WO₄/SiO₂ catalyst in the absence of CH₃Cl in the feed. The (Sb₂O₃)—Mn—Na₂WO₄/SiO₂ catalyst displays an initial O₂ conversion of about 100%, and after 10 hours on stream the O₂ conversion starts to decrease indicating a decline in catalyst activity.

FIG. 3 displays the O₂ conversion for the Mn—Na₂WO₄/SiO₂ catalyst in the absence of CH₃Cl in the feed. The Mn—Na₂WO₄/SiO₂ catalyst displays an initial O₂ conversion of about 98%, which continuously decreases indicating a decline in catalyst activity.

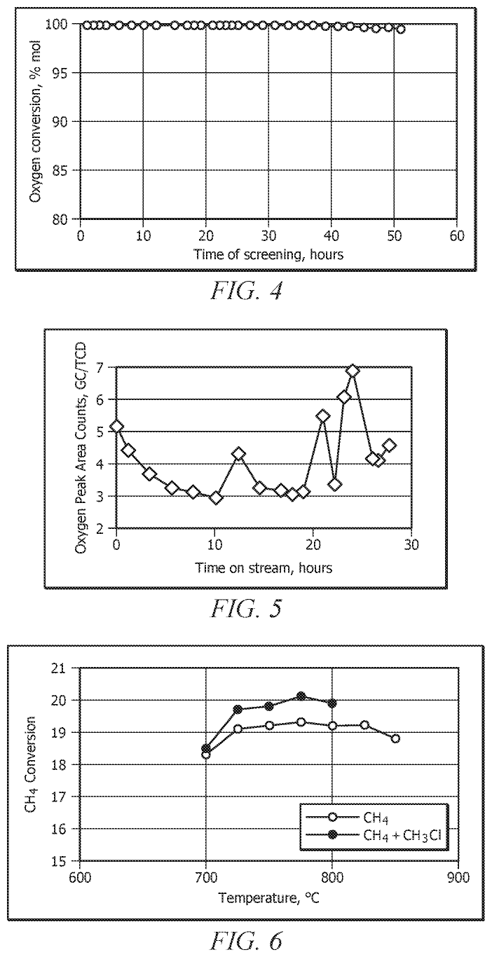

FIG. 4 displays the O₂ conversion for the Mn—Na₂WO₄/SiO₂ catalyst in the presence of 20 ppm CH₃Cl in the feed. The Mn—Na₂WO₄/SiO₂ catalyst displays about 100% O₂ conversion for the entire time on stream (TOS) with no apparent decline in catalyst activity.

FIG. 5 displays the raw data for the oxygen peak area measurements for the Mn—Na₂WO₄/SiO₂ catalyst in the presence of 20 ppm CH₃Cl in the feed.

As will be appreciated by one of skill in the art, and with the help of this disclosure, and without wishing to be limited by theory, generally deactivation takes place when a metal oxide phase agglomerates (if there is no evaporation of metal). Without wishing to be limited by theory, the change of O₂ area count can be due to the oxidation-reduction cycles of the metal oxide (MOₓ₃). When MOₓ₃ agglomerates, HCl (formed from CH₃Cl decomposition) can transform agglomerates to an oxy-chloride phase; and this process can alternate during the accumulation of various phases, which in turn leads to the improved stability of the catalyst.

As can be seen from FIGS. 1-5, the addition of 20 ppm CH₃Cl to the feed leads to an improvement in catalyst activity and stability over time for both the Mn—Na₂WO₄/SiO₂ catalyst and the (Sb₂O₃)—Mn—Na₂WO₄/SiO₂ catalyst.

### La—Ce Catalysts (Laₐ₂Ce_{b2}Oₓ₂)

### Example 3

Example 3 describes screening of a La—Ce Catalyst 1 having a La/Ce weight ratio of 16:1 in quartz reactor with an inner diameter (ID) of 2.3 mm with a catalyst loading 21 mg. The flow rates of the gases were 34.8 cc/min CH₄, 4.7 cc/min O₂, and 0.5 cc/min N₂. The methane feed contained 20 ppm CH₃Cl. The La—Ce Catalyst 1 with the La/Ce weight ratio of 16:1 was prepared by precipitation from nitrate salts of these elements (La, Ce) using NH₄OH. The precipitated gel was dried at 90° C. (for 12 hours) and calcined at 650° C. (for 6 hours). The results of this Example 3 are provided in Table 1.

TABLE 1
Results from Example 3

| Catalyst/Temp. | 600° C. | 650° C. | 700° C. | 750° C. | 800° C. |
|---|---|---|---|---|---|
| CH₄ Conversion: | | | | | |
| La—Ce | 16.8 | 16.8 | 16.8 | 16.3 | 15.6 |
| La—Ce + CH₃Cl | 12.7 | 16.4 | 17.7 | 17.4 | 16.6 |
| La—Ce + CH₃Cl - | 11.4 | 15.9 | 17.4 | 17.27 | 16.6 |
| After Conditioning | | | | | |
| After Second | 10.6 | 15.4 | 18.5 | 18.1 | 17.3 |
| Conditioning | | | | | |
| C₂₊ Selectivity: | | | | | |
| La—Ce | 62.5 | 64.5 | 62.8 | 59.9 | 54.7 |
| La—Ce + CH₃Cl | 46.4 | 61.5 | 66.7 | 66.3 | 62.9 |
| La—Ce + CH₃Cl - | 42.0 | 61.0 | 67.5 | 67.8 | 64.8 |
| After Conditioning | | | | | |
| After Second | 30.8 | 55.0 | 66.0 | 66.7 | 64.0 |
| Conditioning | | | | | |
| O₂ Conversion: | | | | | |
| La—Ce | 96.6 | 100 | 100 | 100 | 100 |
| La—Ce + CH₃Cl | 80.4 | 94.6 | 99.3 | 100 | 100 |
| La—Ce + CH₃Cl - | 76.3 | 93.9 | 99.5 | 100 | 100 |
| After Conditioning | | | | | |
| After Second | 70.6 | 89.1 | 98.8 | 100 | 100 |
| Conditioning | | | | | |
| Volumetric Ratio | | | | | |
| (v/v)CO/CO₂: | | | | | |
| La—Ce | 0.25 | 0.21 | 0.23 | 0.28 | 0.42 |
| La—Ce + CH₃Cl | 1.31 | 0.86 | 0.57 | 0.41 | 0.37 |
| La—Ce + CH₃Cl - | 1.58 | 1.0 | 0.42 | 0.34 | 0.33 |
| After Conditioning | | | | | |
| After Second | 1.9 | 1.5 | 0.70 | 0.47 | 0.37 |
| Conditioning | | | | | |
| Volumetric Ratio | | | | | |
| (v/v)C₂H₄/C₂H₆ | | | | | |
| La—Ce | 0.72 | 0.78 | 0.80 | 0.90 | 1.09 |
| La—Ce + CH₃Cl | 0.35 | 0.68 | 0.90 | 0.86 | 0.98 |
| La—Ce + CH₃Cl - | 0.39 | 0.57 | 0.80 | 0.87 | 0.96 |
| After Conditioning | | | | | |
| After Second | 0.23 | 0.62 | 0.82 | 0.88 | 1.0 |
| Conditioning | | | | | |

### Example 4

Example 4 describes screening of a La—Ce Catalyst 2 having a weight ratio of La/Ce of 16:1 in a quartz reactor with an inner diameter (ID) of 2.3 mm with a La—Ce Catalyst 2 loading of 21 mg. The flow rates of the gases were 34.8 cc/min CH₄, 4.7 cc/min O₂, and 0.5 cc/min N₂. The methane feed contained 5 ppm CH₃Cl. The results of Example 4 are provided in Table 2.

TABLE 2
Results from Example 4

| Catalyst/Temp. | 600° C. | 650° C. | 700° C. | 750° C. | 800° C. |
|---|---|---|---|---|---|
| Selectivity to C₂: | | | | | |
| La—Ce | 59.2 | 62.9 | 63.7 | 61.7 | 56.9 |
| La—Ce + 5 ppm CH₃Cl | 50.7 | 62.5 | 66.4 | 64.3 | 61.8 |
| Selectivity to CO: | | | | | |
| La—Ce | 8.8 | 6.7 | 6.5 | 7.7 | 10.7 |
| La—Ce + 5 ppm CH₃Cl | 24.6 | 14.6 | 9.4 | 8.4 | 8.3 |
| Selectivity to CO₂: | | | | | |
| La—Ce | 31.9 | 30.4 | 29.7 | 30.5 | 32.3 |
| La—Ce + 5 ppm CH₃Cl | | | | | |
| | 24.6 | 22.8 | 24.2 | 27.3 | 29.8 |
| CH₄ Conversion: | | | | | |
| La—Ce | 15.5 | 16.7 | 16.7 | 16.4 | 15.4 |
| La—Ce + 5 ppm CH₃Cl | 14.5 | 17.1 | 17.5 | 17.4 | 16.2 |
| O₂ Conversion: | | | | | |
| La—Ce | 98.2 | 99.1 | 100 | 100 | 100 |
| La—Ce + 5 ppm CH₃Cl | 88.3 | 97.2 | 99.7 | 100 | 100 |
| Volumetric Ratio | | | | | |
| (v/v)C₂H₄/C₂H₆: | | | | | |
| La—Ce | 0.65 | 0.74 | 0.78 | 0.85 | 1.01 |
| La—Ce + 5 ppm CH₃Cl | 0.47 | 0.69 | 0.80 | 0.86 | 0.97 |

As Examples 3 and 4 (and Tables 1 and 2, respectively) show, the addition of less than 20 ppm (e.g., 4-16 ppm) CH₃Cl to the reactant mixture leads to an improvement of selectivity to C₂₊ hydrocarbons of the OCM process. It is important to note that the improvement of selectivity take place in the high temperature area (e.g., at temperatures greater than 600° C., 650° C., 700° C., 750° C., or 800° C.), which is important from a process point of view while at adiabatic reactor conditions at reaction temperatures about 800° C. the presence of CH₃Cl in the reactant mixture as described herein can be utilized to eliminate the drop of selectivity conventionally observed.

### Srₐ₁La_{b1}E_{c1}D_{d1}Oₓ₁ Catalysts

### Example 5

Example 3 describes the screening of the La—Ce—O catalyst (with a weight ratio of La to Ce of 16), Comparative Catalyst 1 or CC1, in a quartz reactor with an internal diameter (ID) of 2.3 mm with a catalyst loading of 21 mg. The flow rates of the gases were 34.8 cc/min CH₄ and 4.7 cc/min of O₂, and 0.5 cc/min of N₂. The methane feed in this Example 5 contained 20 ppm CH₃Cl.

Catalyst CC1 was prepared by precipitation from lanthanum and cerium nitrate salts of these elements (La, Ce) using NH₄OH; in a manner similar to the preparation of La—Ce Catalyst 1 in Example 2. The precipitated gel was dried at 90° C. (for 12 hours) and calcined at 900° C. (for 4 hours). The results of this Example 5 are provided in Table 3.

Table 3 displays the performance of La—Ce catalyst CC1 after replacement of CH₄ feed to CH₄ plus 20 ppm CH₃Cl at gas flow rates of 34.7 cc/min CH₄, 4.7 cc/min of O₂ (for a volumetric ratio of CH₄ to O₂ of 7.4), and 0.5 cc/min of N₂ utilizing 21 mg catalyst, in an OCM reactor comprising a tube having an inner diameter (ID) of 2.3 mm.

TABLE 3
Results from Example 5

| | |
|---|---|
| | After replacement of |
| Standard- | CH₄ with CH₄ + CH₃Cl |

| | No CH₃Cl | 850° C. | 850° C. | 850° C. |
|---|---|---|---|---|
| | 850° C. | 10 min | 20 min | 30 min |
| C₂ Selectivity | 52.3 | 57.4 | 57.6 | 57.9 |
| CH₄ Conversion | 15.1 | 15.7 | 15.8 | 15.8 |
| O₂ Conversion | 100 | 100 | 100 | 100 |
| CO Selectivity | 14.3 | 12.1 | 12.0 | 11.8 |
| CO₂ Selectivity | 33.4 | 30.5 | 30.3 | 30.2 |

### Example 6

Example 6 describes investigating the Sr—La—Yb—Nd—O Catalyst 1, in a quartz reactor with an ID of 2.3 mm with a catalyst loading of 21 mg. The flow rates of the gases were 34.8 cc/min of CH₄, 4.7 cc/min of O₂, and 0.5 cc/min of N₂. The methane feed in this Example 6 contained 20 ppm CH₃Cl. Sr—La—Yb—Nd—O Catalyst 1 was prepared by precipitation from nitrate salts of these elements (Sr, La, Yb, Nd) with using NH₄OH. The precipitated gel was dried at 90° C. (for 12 hours) and calcined at 900° C. (for 4 hours). The results of Example 6 are provided in Table 4.

Table 4 displays the performance of the Sr—La—Yb—Nd—O Catalyst 1 after replacement of CH₄ feed to CH₄ plus 20 ppm CH₃Cl with gas flow rates of 34.7 cc/min CH₄, 4.7 cc/min O₂ (for a volumetric ratio of CH₄ to O₂ of 7.4), and 0.5 cc/min N₂, with 21 mg catalyst in a tube reactor having an inner diameter (ID) of 2.3 mm.

TABLE 4
Results from Example 6

| | Standard- | | After replacement of CH₄ with CH₄ + CH₃Cl | | | | | |
|---|---|---|---|---|---|---|---|---|
| | No CH₃Cl | | 850° C. | 850° C. | 850° C. | 850° C. | 850° C. | Back to |
| | 800° C. | 850° C. | 15 min | 30 min | 45 min | 60 min | 120 min | 800° C. |
| C₂ Selectivity | 71.7 | 68.9 | 71.1 | 71.2 | 71.6 | 72.1 | 72.1 | 75.1 |
| CH₄ Conversion | 18.1 | 17.4 | 17.8 | 17.8 | 17.9 | 18.1 | 18.0 | 19.0 |
| O₂ Conversion | 100 | 100 | 100 | 100 | 100 | 100 | 100 | 100 |
| CO Selectivity | 3.5 | 4.27 | 3.50 | 3.40 | 3.42 | 3.42 | 3.39 | 3.16 |
| CO₂ Selectivity | 24.2 | 26.7 | 25.4 | 25.3 | 25.0 | 24.5 | 24.5 | 21.7 |

### Example 7

Example 7 describes the performance of the Sr—La—Yb—Nd—O Catalyst 1 of Example 6 on the second day in the quartz reactor with the ID 2.3 mm and with the catalyst loading of 0.21 mg. Again, the flow rates of the gases were 34.8 cc/min of CH₄, 4.7 cc/min of O₂, and 0.5 cc/min of N₂. The methane feed contained 20 ppm CH₃Cl. The results of this Example 7 are shown in Table 5.

Table 5 displays the second day performance of the Sr—La—Yb—Nd—O Catalyst 1 after replacement of a CH₄ feed with a feed comprising CH₄ plus 20 ppm CH₃Cl with gas flow rates of 34.7 cc/min CH₄, 4.7 cc/min O₂ (for a volumetric ratio of CH₄ to O₂ of 7.4), and 0.5 cc/min of N₂ in a 2.3 mm tube reactor comprising 21 mg of the Sr—La—Yb—Nd—O Catalyst 1.

TABLE 5
Results from Example 7

| | | | Second Day | | | | | |
|---|---|---|---|---|---|---|---|---|
| | First Day | First Day | Standard Expt. | Second Day After Replacement of | | | | |
| | Standard Expt. | Standard Expt. | 850° C. | CH₄ with CH₄ + CH₃Cl | | | | |
| | 800° C. -No | 850° C. -No | After CH₃Cl | 850° C. | 850° C. | 850° C. | Back to 800° C. | |
| | CH₃Cl | CH₃Cl | Feed- No CH₃Cl | 40 min | 55 min | 85 min | 15 min | 30 min |
| C₂ | 71.7 | 68.9 | 70.1 | 72.6 | 72.7 | 72.6 | 75.5 | 75.5 |
| Selectivity | | | | | | | | |
| CH₄ | 18.1 | 17.4 | 17.8 | 18.4 | 18.3 | 18.4 | 19.2 | 19.3 |
| Conversion | | | | | | | | |
| O₂ | 100 | 100 | 100 | 100 | 100 | 100 | 100 | 100 |
| Conversion | | | | | | | | |
| CO | 3.5 | 4.27 | 3.89 | 3.34 | 3.39 | 3.49 | 3.32 | 3.33 |
| Selectivity | | | | | | | | |
| CO₂ | 24.2 | 26.7 | 25.9 | 24.1 | 23.9 | 23.8 | 21.18 | 21.18 |
| Selectivity | | | | | | | | |

### Example 8

Example 8 describes a repeat of experiments with a new portion of Sr—La—Yb—Nd—O Catalyst 1 in a quartz reactor having a 2.3 mm ID and a catalyst loading of 21 mg. The flow rates of the gases were 34.8 cc/min CH₄, 4.7 cc/min O₂, and 0.5 cc/min N₂. The methane feed in this Example 8 contained 20 ppm CH₃Cl. The results of this Example 8 are shown in Table 6.

Table 6 displays the experimental results for a new portion of Sr—La—Yb—Nd—O Catalyst 1 and repeat of experiments with replacement of CH₄ feed with reactant mixture feed comprising CH₄ and 20 ppm CH₃Cl, with gas flow rates of 34.7 cc/min of CH₄, 4.7 cc/min of O₂ (for a volumetric ratio of CH₄ to O₂ of 7.4), and 0.5 cc/min of N₂, with a 2.3 mm tube reactor containing 21 mg of the Sr—La—Yb—Nd—O Catalyst 1.

TABLE 6
Results from Example 8

| | Standard Expt.- | | After replacement of CH₄ with | | | Return Back to |
|---|---|---|---|---|---|---|
| | No CH₃Cl | | CH₄ + CH₃Cl Feed; 880° C. | | | 750° C. |
| | 750° C. | 880° C. | 15 min | 25 min | 35 min | 15 min |
| C₂ | 73.2 | 69.3 | 71.1 | 71.1 | 71.3 | 76.4 |
| Selectivity | | | | | | |
| CH₄ | 18 8 | 17.6 | 17.7 | 17.8 | 17.8 | 19.7 |
| Conversion | | | | | | |
| O₂ | 100 | 100 | 100 | 100 | 100 | 100 |
| Conversion | | | | | | |
| CO | 3.4 | 4.8 | 4.1 | 4.0 | 4.1 | 3.9 |
| Selectivity | | | | | | |
| CO₂ | 23.3 | 25.8 | 24.8 | 24.8 | 24.6 | 19.7 |
| Selectivity | | | | | | |

### Example 9

Example 9 describes experiments with a new portion of Sr—La—Yb—Nd—O Catalyst 1 in a quartz reactor having an inner diameter (ID) of 2.3 mm containing a catalyst loading of 21 mg, where the temperature is decreased to 650° C. after attaining a temperature of 850° C. The flow rates of the gases in this Example 9 were 34.8 cc/min of CH₄, 4.7 cc/min of O₂, and 0.5 cc/min of N₂. The methane feed contained 20 ppm CH₃Cl. The results of this Example 9 are shown in Table 7.

Table 7 displays the performance of the Sr—La—Yb—Nd—O Catalyst 1 with replacement of the CH₄ feed with a feed containing CH₄ plus 20 ppm CH₃Cl and reducing the temperature to 650° C.; 34.7 cc/min CH₄, 4.7 cc/min O₂ (for a volumetric ratio of CH₄ to O₂ of 7.4), and 0.5 cc/min N₂, in a reactor comprising a 2.3 mm tube and 21 mg Sr—La—Yb—Nd—O Catalyst 1.

TABLE 7
Results from Example 9

| | Standard Expt. 650° C. | CH₄ + CH₃Cl Feed; |
|---|---|---|
| | First Day -No CH₃Cl | After 850° C. Back to 650° C. |
| C₂ Selectivity | 70.9 | 70.5 |
| CH₄ | 18.4 | 17.5 |
| Conversion | | |
| O₂ Conversion | 99.9 | 90.3 |
| CO Selectivity | 5.6 | 10.3 |
| CO₂ Selectivity | 23.49 | 19.3 |

As the results from Examples 5-9 show, the addition of chlorine salts to the Sr—La—Yb—Nd—O catalyst increases the selectivity of the OCM process. It is important to note that the improvement of selectivity takes place in the high temperature area (e.g., at temperatures greater than 600° C., 650° C., 700° C., 750° C., or 800° C.), which is important from a process point of view; while at adiabatic reactor conditions at reaction temperatures about 800° C. the presence of chlorine in the reactant mixture as described herein can be utilized to eliminate the drop of selectivity conventionally observed.

### Example 10

Example 10 describes experiments with a new portion of Sr—La—Yb—Nd—O Catalyst 1 in a quartz reactor with an ID of 2.3 mm with a catalyst loading of 21 mg. The flow rates of the gases were 34.8 cc/min of CH₄, 4.7 cc/min of O₂, and 0.5 cc/min of N₂. The methane feed in this Example 10 contained 20 ppm CH₃Cl. The results of Example 10 are displayed in FIGS. 6-8.

FIG. 6 displays the effect of 20 ppm amount of CH₃Cl in the feed on the CH₄ conversion; wherein the CH₄ conversion is increased in the presence of 20 ppm CH₃Cl in the feed, over the temperature range investigated.

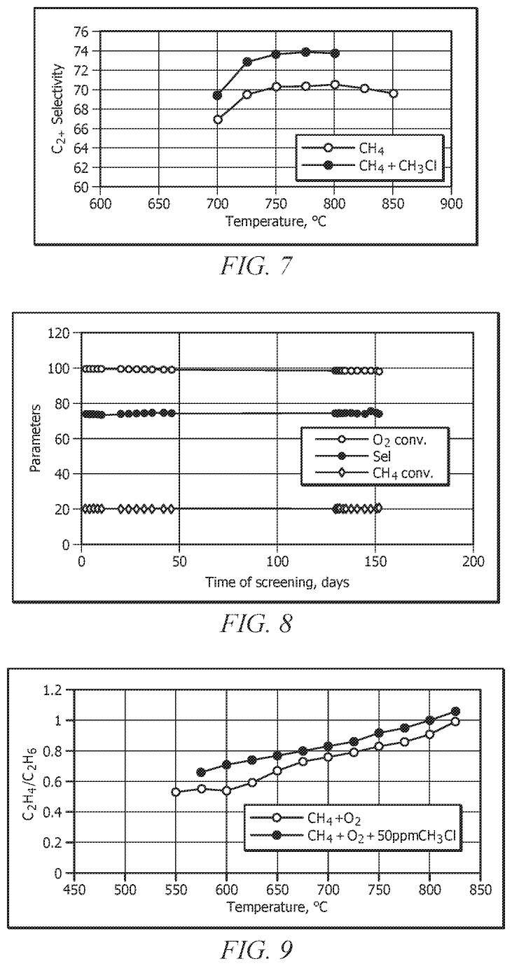

FIG. 7 displays the effect of 20 ppm amount of CH₃Cl in the feed on the C₂₊ selectivity; wherein the C₂₊ selectivity is increased in the presence of 20 ppm CH₃Cl in the feed, over the temperature range investigated.

FIG. 8 displays a long term stability test of Sr—La—Yb—Nd—O Catalyst 1 in the presence of 20 ppm amount of CH3Cl in the feed; wherein the CH₄ conversion, the O₂ conversion, and the C₂₊ selectivity are substantially stable for a time period of greater than 150 hours.

### Example 11

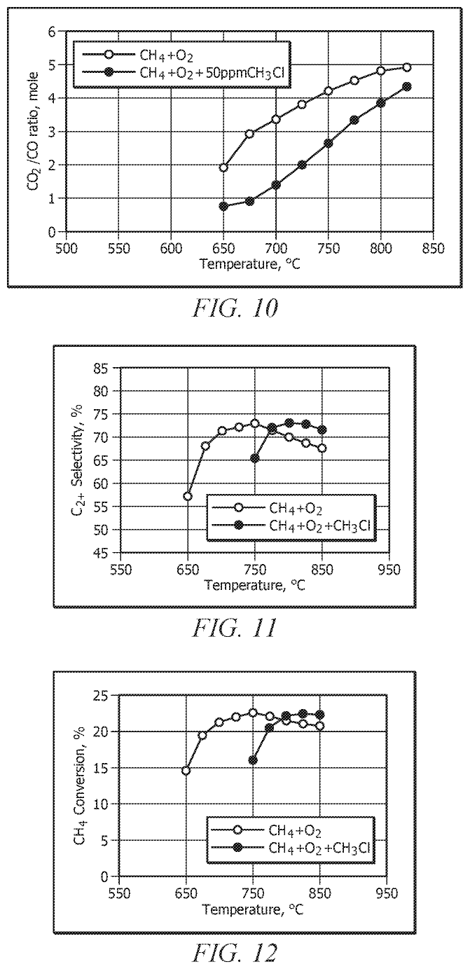

Example 11 describes investigating the Sr—La—Yb—Nd—O Catalyst 2 (unsupported Sr—La—Yb—Nd—O Catalyst 2; unsupported Sr_{0.1}La_{0.9}Nd_{0.7}Yb_{0.1}Oₓ₁), in a quartz reactor with an ID of 2.3 mm with a catalyst loading of 21 mg. The flow rates of the gases were 37.5 cc/min of CH₄, 4.7 cc/min of O₂, and 0.5 cc/min of N₂. The methane feed in this Example 11 contained 50 ppm CH₃Cl. Sr—La—Yb—Nd—O Catalyst 2 was prepared by precipitation from nitrate salts of these elements (Sr, La, Yb, Nd) with using NH₄OH. The precipitated gel was dried at 90° C. (for 12 hours) and calcined at 900° C. (for 6 hours). The results of Example 11 are displayed Tables 8 and 9, as well as in FIGS. 9 and 10.

TABLE 8

| | C₂ Selectivity, % mole | | |
|---|---|---|---|
| | | CH₄ + O₂ + 50 | CH₄ + O₂ + 50 |
| T, ° C. | CH₄ + O₂ | ppmCH₃Cl | ppmCH₃Cl (repeat) |
| 575 | 64.5 | 69.0 | 68.0 |
| 600 | 64.2 | 69.4 | 69.2 |
| 625 | 64.1 | 70.4 | 70.5 |
| 650 | 66.7 | 72.4 | 71.8 |
| 675 | 68.6 | 72.2 | 71.5 |
| 700 | 69.4 | 72.4 | 72.0 |
| 725 | 69.6 | 72.1 | 72.1 |
| 750 | 69.3 | 72.8 | 72.2 |
| 775 | 68.9 | 71.7 | 71.7 |
| 800 | 67.5 | 70.1 | 70.5 |
| 825 | 66.3 | 68.5 | 69.0 |

TABLE 9

CH₄ Conversion, % mol

| T, ° C. | CH₄ + O₂ | CH₄ + O₂ + 50 ppmCH₃Cl |
|---|---|---|
| 550 | 19.4 | 19.3 |
| 575 | 19.5 | 20.4 |
| 600 | 20 | 21.3 |
| 625 | 19.4 | 20.9 |
| 650 | 20.1 | 21.1 |
| 675 | 20.7 | 20.9 |
| 700 | 20.9 | 21.1 |
| 725 | 20.9 | 21.7 |
| 750 | 20.7 | 21.6 |
| 775 | 20.6 | 20.6 |
| 800 | 20.1 | 20.7 |
| 825 | 19.6 | 20.5 |

Table 1 shows that addition of methyl chloride in amount of 50 ppm to a mixture of CH₄ and O₂ leads to the increase of C₂ selectivity of about 4-6%. Table 2 shows that CH₄ conversion in the presence of methyl chloride and in the absence of methyl chloride in the reactant mixture. Table 2 indicates that in the presence of methyl chloride in the reactant mixture, CH₄ conversion also increases, along with the increase of C₂ selectivity.

FIG. 9 displays a comparison of C₂H₄/C₂H₆ molar ratio for reactions mixtures without chloride (CH₄+O₂) and with chloride (CH₄+O₂+50 ppm CH₃Cl). FIG. 10 displays a comparison of CO₂/CO molar ratio for reactions mixtures without chloride (CH₄+O₂) and with chloride (CH₄+O₂+50 ppm CH₃Cl). As can be seen from FIG. 10, addition of methyl chloride reduces the CO₂/CO molar ratio in the products, which is a positive effect for controlling the reaction heat, by reducing the heat that would be produced in the highly exothermic methane oxidation to CO₂. FIG. 9 displays the additional positive effect of increasing the C₂H₄/C₂H₆ molar ratio in the products.

### Example 12

Example 12 describes investigating the supported Sr—La—Yb—Nd—O Catalyst 2 (silica-alumina supported Sr_{0.1}La_{0.9}Nd_{0.7}Yb_{0.1}Oₓ₁), in a quartz reactor with an ID of 2.3 mm. The flow rates of the gases were 37.5 cc/min of CH₄, 4.7 cc/min of O₂, and 0.5 cc/min of N₂. The methane feed in this Example 12 contained 50 ppm CH₃Cl. Sr—La—Yb—Nd—O Catalyst 2 was prepared by precipitation from nitrate salts of these elements (Sr, La, Yb, Nd) with using NH₄OH, in the presence of a silica-alumina support. The precipitated material was dried at 90° C. (for 12 hours) and calcined at 900° C. (for 6 hours). The results of Example 12 are displayed in Tables 10-15.

TABLE 10

C₂ Selectivity, % mol

| Temperature, ° C. | CH₄ + O₂ | CH₄ + O₂ + CH₃Cl |
|---|---|---|
| 650 | 50.1 | |
| 675 | 58.7 | |
| 700 | 64.4 | 56.4 |
| 725 | 67.7 | 67.5 |
| 750 | 71.0 | 74.1 |
| 775 | 72.3 | 74.7 |
| 800 | 72.8 | 74.2 |

TABLE 11

Methane conversion, % mol

| Temperature, ° C. | CH₄ + O₂ | CH₄ + O₂ + CH₃Cl |
|---|---|---|
| 650 | 12.7 | |
| 675 | 15.2 | |
| 700 | 17.3 | 13.6 |
| 725 | 18.3 | 16.8 |
| 750 | 19.2 | 19.2 |
| 775 | 19.7 | 19.7 |
| 800 | 19.7 | 19.7 |

TABLE 12

O₂ conversion, % mol

| Temperature, ° C. | CH₄ + O₂ | CH₄ + O₂ + CH₃Cl |
|---|---|---|
| 650 | 75.8 | |
| 675 | 85.0 | |
| 700 | 89.8 | 66.5 |
| 725 | 93.0 | 78.9 |
| 750 | 96.4 | 91.3 |
| 775 | 96.3 | 94.1 |
| 800 | 96.9 | 96.9 |

The data in Tables 10-12 was acquired for a catalyst loading of 20 mg, wherein the active catalytic material was 35%, for an active catalyst loading of 7 mg. Owing to a lower number of active sites in the catalyst, a lower selectivity, as well as a lower methane conversion and O₂ conversion was obtained under otherwise similar conditions at lower temperatures.

TABLE 13

C₂ Selectivity, % mol

| Temperature, ° C. | CH₄ + O₂ | CH₄ + O₂ + CH₃Cl |
|---|---|---|
| 600 | 34.0 | |
| 625 | 53.8 | |
| 650 | 64.5 | 31.1 |
| 675 | 68.8 | 41.3 |
| 700 | 70.5 | 53.9 |
| 725 | 72.3 | 65.7 |
| 750 | 72.8 | 73.1 |
| 775 | 72.7 | 74.6 |
| 800 | 72.4 | 75.0 |
| 825 | 71.3 | 74.4 |

TABLE 14

Methane conversion, % mol

| Temperature, ° C. | CH₄ + O₂ | CH₄ + O₂ + CH₃Cl |
|---|---|---|
| 600 | 11.2 | |
| 625 | 16.5 | |
| 650 | 20.0 | 6.9 |
| 675 | 21.4 | 9.1 |
| 700 | 21.9 | 12.7 |
| 725 | 22.4 | 18.0 |
| 750 | 22.2 | 22.3 |
| 775 | 21.9 | 22.9 |
| 800 | 21.9 | 22.8 |
| 825 | 21.2 | |

TABLE 15

O₂ conversion, % mol

| Temperature, ° C. | CH₄ + O₂ | CH₄ + O₂ + CH₃Cl |
|---|---|---|
| 600 | 75.5 | |
| 625 | 88.7 | |
| 650 | 96.2 | 50.4 |
| 675 | 98.7 | 56.1 |
| 700 | 99.5 | 65.5 |
| 725 | 99.8 | 79.2 |
| 750 | 99.8 | 91.5 |
| 775 | 99.9 | 97.9 |
| 800 | 99.9 | 99.4 |
| 825 | 99.9 | |

The data in Tables 13-15 was acquired for a catalyst loading of 60 mg, wherein the active catalytic material was 35%, for an active catalyst loading of 21 mg.

As can be seen by comparing the data in Tables 8 and 9 with the data in Tables 10 and 11: in the presence of bulk catalyst Sr—La—Yb—Nd—O (unsupported Sr—La—Yb—Nd—O Catalyst 2, Tables 8 and 9) the positive effect of CH₃Cl takes place at all investigated temperatures, while in the presence of a catalyst supported on an alumino-silicate (supported Sr—La—Yb—Nd—O Catalyst 2, Tables 10 and 11) good performance in the presence of CH₃Cl in the reactant mixture was observed only at relatively high temperature, which could be due to the low content of the active material. However, experimental results with loading more catalyst (60 mg, data in Tables 13 and 14) indicated that the positive effect was also observed only at relatively high temperatures. Thus, the data in Examples 11 and 12 indicates that unsupported catalysts displays positive effect of CH₃Cl at all investigated temperatures, while supported catalysts displays positive effect of CH₃Cl at relatively high temperature ranges (e.g., greater than about 750° C.).

The data in Examples 11 and 12 indicates that the positive effect of methyl chloride on the C₂ selectivity of for the unsupported (bulk) Sr—La—Yb—Nd—O Catalyst 2 can be observed in all investigated ranges of reaction temperatures, such as 575° C.-825° C., even as low as 550° C.

### Example 13

Example 13 describes investigating the supported Sr—La—Yb—Nd—O Catalyst 3 (alpha-alumina supported Sr—La—Yb—Nd—O), in a quartz reactor with an ID of 2.3 mm with a catalyst loading of 21 mg. The flow rates of the gases were 37.5 cc/min of CH₄, 4.7 cc/min of O₂, and 0.5 cc/min of N₂. The methane feed in this Example 13 contained 50 ppm CH₃Cl. Supported Sr—La—Yb—Nd—O Catalyst 3 was prepared by precipitation from nitrate salts of these elements (Sr, La, Yb, Nd) with using NH₄OH, in the presence of an alpha-alumina support. The precipitated material was dried at 90° C. (for 12 hours) and calcined at 900° C. (for 6 hours). The results of Example 13 are displayed in FIGS. 11-13.

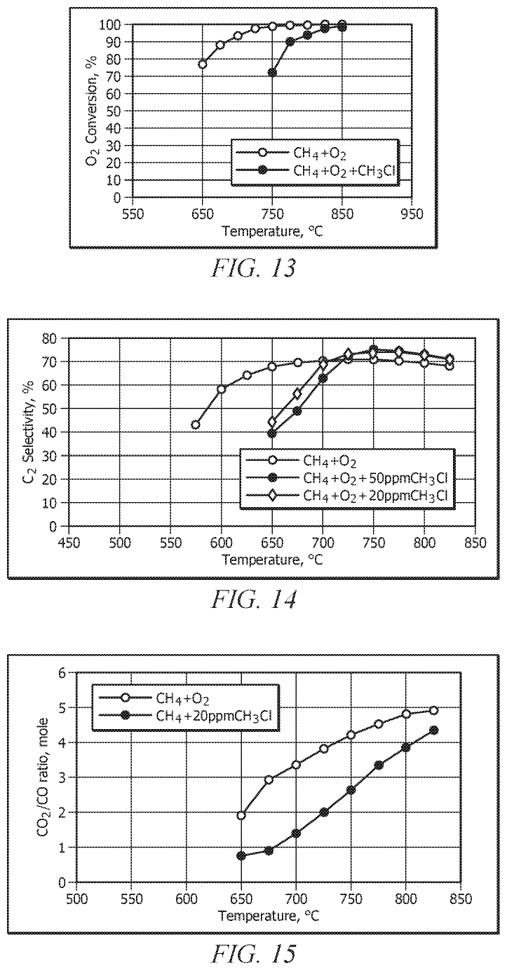

FIG. 13 displays the effect of CH₃Cl on C₂₊ selectivity. At low temperatures, CH₃Cl has a negative effect, but at temperatures above 775° C., C₂₊ selectivity increases. Further, at 850° C. selectivity increases from 67.7% (in the absence of the CH₃Cl) to 71.7% (in the presence of the CH₃Cl). Without wishing to be limited by theory, in highly exothermic methane oxidative coupling reaction, it is known that at methane conversions more than 15%, the reaction temperature increases to the level about 850° C.-900° C.; and runaway of the temperature to this range leads to the drop of C₂₊ selectivity. Consequently, an increase in selectivity at relatively high temperatures by addition of methyl chloride is a positive feature of CH₃Cl addition to the OCM feed as disclosed herein.

FIG. 14 displays the effect of CH₃Cl on the CH₄ conversion, wherein CH₃Cl addition to the OCM feed has a similar effect to the effect of CH₃Cl addition on the C₂₊ selectivity. At low temperatures, CH₃Cl has a negative effect, but at temperatures above 775° C., CH₄ conversion increases. Further, FIG. 15 displays the effect of CH₃Cl on the O₂ conversion, wherein CH₃Cl addition to the OCM feed has a similar effect to the effect of CH₃Cl addition on the C₂₊ selectivity. At low temperatures, CH₃Cl has a negative effect, but at temperatures above 775° C., O₂ conversion increases.

### Srₐ₁La_{b1}Oₓ₁/MgO Catalysts

### Example 14

Example 14 describes investigating the supported Sr—La—MgO Catalyst 4 (MgO supported Sr—La—O or Srₐ₁La_{b1}Oₓ₁/MgO) having the general formula Sr_{1.0}La_{0.18}Mg_{0.13}Oₓ₁, in a quartz reactor with an ID of 2.3 mm with a catalyst loading of 21 mg. The flow rates of the gases were 37.5 cc/min of CH₄, 4.7 cc/min of O₂, and 0.5 cc/min of N₂. The methane feed in this Example 14 contained 20 ppm CH₃Cl or 50 ppm CH₃Cl. Supported Sr—La—MgO Catalyst 4 was prepared by slow evaporation of an aqueous solution containing nitrate salts of these elements (Sr, La) at a temperature of 70° C., in the presence of a MgO support. The precipitated material was dried at 90° C. (for 24 hours) and calcined at 900° C. (for 4 hours). The supported Sr—La—MgO Catalyst 4 was sieved for 20-40 mesh for testing. The results of Example 14 are displayed in Table 16, as well as FIGS. 14 and 15.

TABLE 16

| | C₂ selectivity, % | | |
|---|---|---|---|
| | | CH₄ + O₂ + 20 | CH₄ + O₂ + 50 |
| T, ° C. | CH₄ + O₂ | ppmCH₃Cl | ppmCH₃Cl |
| 700 | 70.5 | 69.3 | 63.2 |
| 725 | 71.2 | 73.3 | 72.7 |
| 750 | 71.1 | 74.2 | 75.1 |
| 775 | 70.5 | 74.2 | 74.5 |
| 800 | 69.6 | 73.1 | 73.1 |
| 825 | 68.4 | 71.1 | 71.4 |

The data in Table 16 indicate that addition of CH₃Cl to the OCM feed leads to the improvement of selectivity, specifically in the relatively high temperature range (e.g., over about 725° C.). The improvement of C₂ selectivity in the relatively high temperature range (e.g., over about 725° C.) is significant from a practical process perspective, given that it provides a solution for reactor operation under adiabatic conditions at a reaction temperature of about 800° C. in the presence of CH₃Cl in the reactant mixture. The data in Table 16 indicate that under conventional conditions (in the absence of CH₃Cl in the reactant mixture), the C₂ selectivity drops with increasing the temperature, while the C₂ selectivity increases with increasing the temperature in the presence of CH₃Cl in the reactant mixture, wherein the presence of CH₃Cl in the reactant mixture eliminates the drop in selectivity with increasing the temperature.

FIG. 14 displays the effect of different levels of CH₃Cl in the OCM feed on the C₂ selectivity. As can be seen, the positive effect of CH₃Cl takes place only at relatively high temperatures above 700° C. and at these relatively high temperatures addition of different amounts of CH₃Cl to the CH₄+O₂ feed leads to similar results displaying an increase in C₂ selectivity with increasing the temperature. However, at temperatures less than 725° C. the addition of methyl chloride leads to a decrease in selectivity to C₂ hydrocarbons. Without wishing to be limited by theory, the result in FIG. 14 may be explained by the blockage of surface by Cl with formation of chloride of basic elements, such as LaCl₃, LaOCl, MgCl₂, and the like, or combinations thereof. Further, and without wishing to be limited by theory, at relatively high temperatures (e.g., over about 725° C.) chlorine is removed into the gas phase in the form of Cl radicals (Cl.), wherein Cl. can participate in gas phase reactions, thereby propagating the OCM reaction and leading to the formation of C₂ hydrocarbons, as previously described herein.

FIG. 15 displays the variation of the CO₂/CO molar ratio with the temperature in the presence (50 ppm) and in the absence of CH₃Cl in the OCM feed. As it ca be seen from FIG. 15, the presence of CH₃Cl in the OCM feed significantly reduces the CO₂/CO molar ratio in the products, which a very important aspect of heat management when conducting an OCM reaction, as previously described herein.

### Example 15

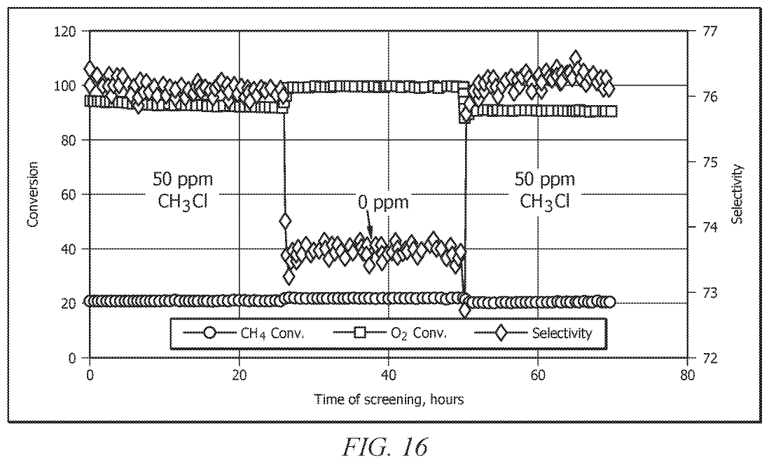

Example 15 describes investigating OCM reactions with a new portion of the supported Sr—La—MgO Catalyst 4 (MgO supported Sr—La—O or Srₐ₁La_{b1}Oₓ₁/MgO) having the general formula Sr_{1.0}La_{0.18}Mg_{0.13}Oₓ₁, in a quartz reactor with an ID of 2.3 mm with a catalyst loading of 20 mg. The flow rates of the gases were 34.8 cc/min of CH₄, 4.7 cc/min of O₂, and 0.5 cc/min of N₂. The methane feed in this Example 15 contained 0 ppm CH₃Cl or 50 ppm CH₃Cl. The experiments in Example 15 were conducted under near iso-thermal conditions at a reaction temperature (e.g., furnace temperature) of 775° C. The results of Example 15 are displayed in FIG. 16.

FIG. 16 indicates that under near iso-thermal conditions at a reaction temperature of 775° C., the C₂₊ selectivity increases more than twice in the presence 50 ppm CH₃Cl, wherein the selectivity is about 100%.

### Example 16

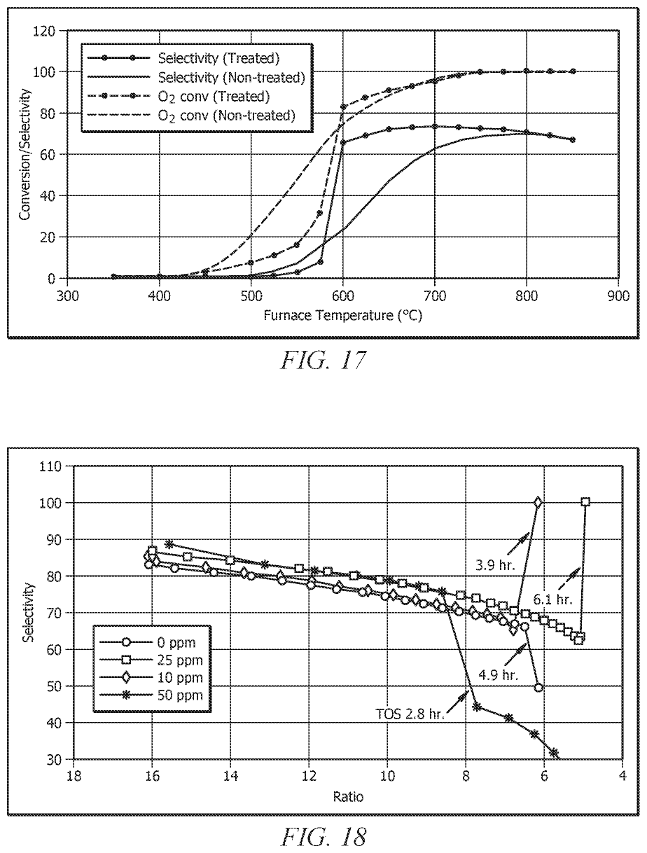

Example 16 describes investigating OCM reactions with a new portion of the supported Sr—La—MgO Catalyst 4 and with a pre-treated supported Sr—La—MgO Catalyst 5 having the general formula Sr_{1.0}La_{0.18}Mg_{0.13}Oₓ₁, that has been prepared by pre-treating the supported Sr—La—MgO Catalyst 4 at room temperature for 8 h with 50 ppm CH₃Cl, in a quartz reactor with an ID of 2.3 mm with a catalyst loading of 20 mg. The flow rates of the gases were 34.8 cc/min of CH₄, 4.7 cc/min of O₂, and 0.5 cc/min of N₂. The methane feed in this Example 16 contained 0 ppm CH₃Cl or 50 ppm CH₃Cl. The results of Example 16 are displayed in FIG. 17.

FIG. 17 indicates that pre-treating the supported Sr—La—MgO Catalyst 4 at room temperature for 8 h with 50 ppm CH₃Cl results in pre-treated supported Sr—La—MgO Catalyst 5 having superior properties over a temperature range of from about 600° C. to about 800° C., both in terms of C₂₊ selectivity and O₂ conversion.

### Example 17

Example 17 describes investigating OCM reactions with a new portion of the supported Sr—La—MgO Catalyst 4 under autothermal reaction conditions, in a quartz reactor with an ID of 10.5 mm with a catalyst loading of 400 mg, and a catalyst bed length of 5 mm. The autothermal OCM reaction in this Example 17 was carried out by transferring heat of reaction to incoming feed in situ without application of external heat exchange devices. The autothermal operation of the OCM reactor was possible by heating the catalyst first, such that the catalyst could be ignited at a certain temperature, for example at 450° C.-550° C., when feed mixture of methane and oxygen was passed over it. After ignition, the external furnace heating was turned off gradually in a programmed fashion. The CH₄+O₂ feed was allowed to decrease as the furnace temperature was ramped down, such that the catalyst bed temperature was maintained in a narrow range to keep the catalyst bed ignited. The procedure of autothermal (ATR) experiments was as follows: (1) the catalyst in the reactor was heated under N₂ and CH₄ to the desired temperature (i.e., 650° C.) by using the reactor furnace; (2) the reactor feed was rapidly switched to CH₄ and O₂ (CH₄/O₂=16) to ignite the catalyst bed; (3) the catalyst reached a steady state temperature between 750° C. and 950° C. (typically 860° C.) in about 1 hour; and (4) autothermal operation protocol was started with the selected CH₃Cl concentration in the feed. In autothermal operation mode, the power delivered to the furnace was linearly decreased to zero within 5 hours. During the cooling process, a target constant bed temperature of 860° C. was maintained by adjusting the CH₄/O₂ molar ratio in the feed starting from an initial CH₄/O₂ ratio of 16. Experiments were repeated with 0 ppm, 10 ppm, 25 ppm and 50 ppm CH₃Cl added to the feed. The results of Example 17 are displayed in FIG. 18.

The data in FIG. 18 indicate that generally, the C₂₊ selectivity is highest at the highest CH₄/O₂ molar ratio, wherein the C₂₊ selectivity has the most elevated values in the presence of CH₃Cl in the feed.

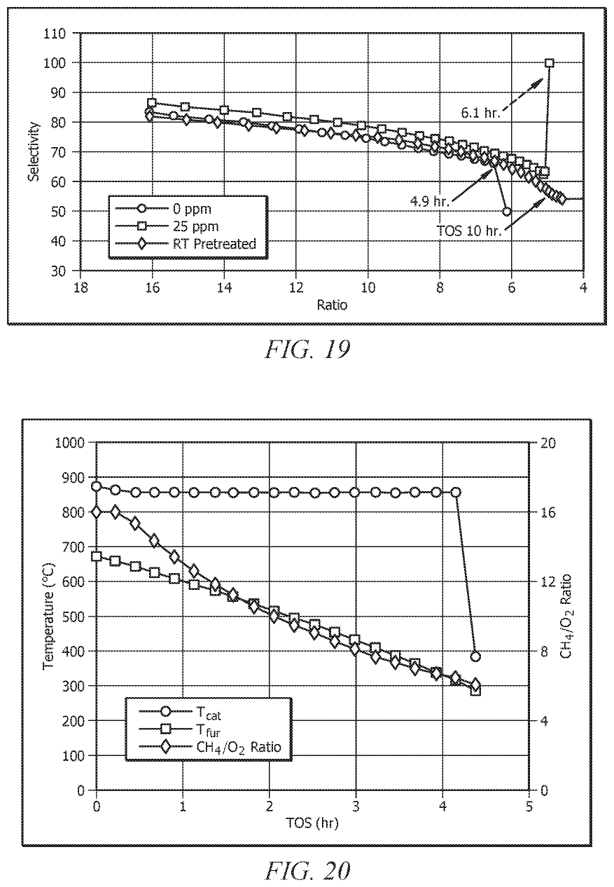

The performance of the supported Sr—La—MgO Catalyst 4 was compared with the performance of the pre-treated supported Sr—La—MgO Catalyst 5 under autothermal conditions as described in this Example 17, and the data is displayed in FIG. 19. The supported Sr—La—MgO Catalyst 4 was investigated under autothermal conditions with (25 ppm) and without (0 ppm) addition of CH₃Cl to the feed during autothermal operation. The pre-treated supported Sr—La—MgO Catalyst 5 was investigated under autothermal conditions without addition of CH₃Cl to the feed during autothermal operation. The room temperature pre-treatment (50 ppm CH₃Cl for 8 h) does not change the catalyst selectivity, but increases activity or productivity (time on stream (TOS) 10 h vs 4.9 h). It must be noted that the reactor extinguishes in the autothermal mode of operation because of excessive loss of heat at laboratory scale. As such, the time for which a catalyst survives autothermal mode of operation is an indirect measure of the activity of the catalyst, i.e., the more active catalyst results in a higher rate of reaction that in turn results in higher rate of heat generation. For a given heat loss in autothermal mode, a catalyst that has higher rate of heat generation will last longer (i.e., it will take longer to extinguish).

The procedure for conducting autothermal (ATR) OCM processes was as follows. The catalyst was heated under N₂ and CH₄ to the desired temperature (e.g., 650° C.), followed by rapidly switching to a feed comprising O₂ and CH₄ with a CH₄/O₂ molar ratio of 16 to ignite the catalyst bed. When the catalyst bed reached a controlled temperature of about 860° C. (within about 1 hour), autothermal operation with various CH₃Cl concentrations in the feed was started. Under the autothermal operation mode, the power delivered to the furnace was linearly decreased to zero within 5 hours. During a cooling process, a constant bed temperature of 860° C. was maintained by adjusting (e.g., decreasing) a fuel ratio (CH₄/O₂ molar ratio) from an initial ratio 16. Experiments were repeated with 0, 10, 25 and 50 ppm CH₃Cl added to the feed.

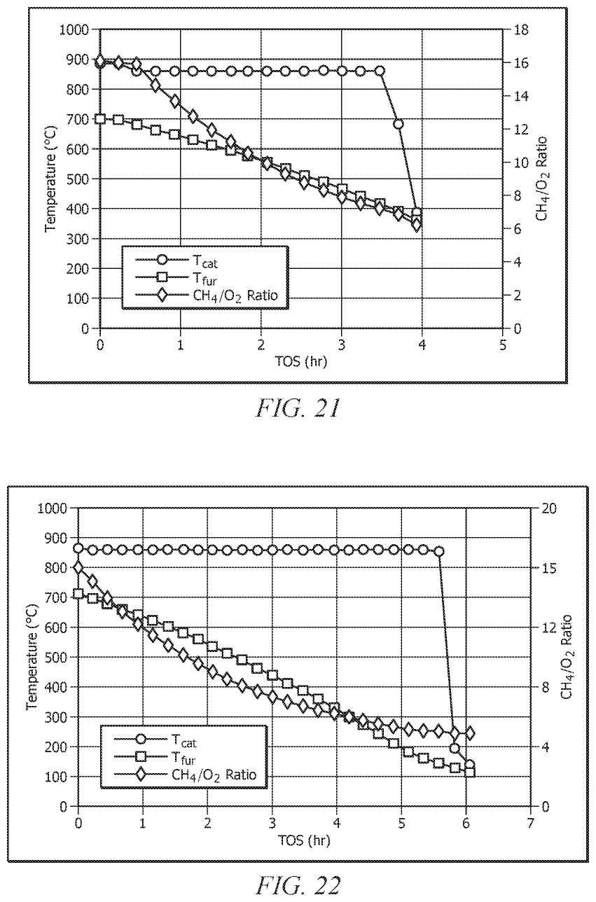

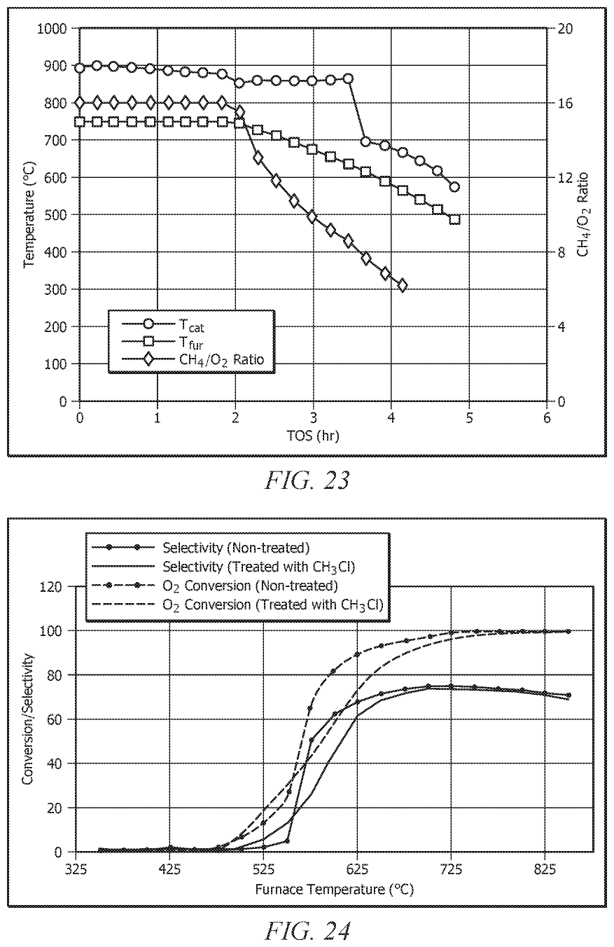

The data in FIGS. 20-23 display the temperature profile of the autothermal reactor in the absence of CH₃Cl in the feed (FIG. 20), as well as in the presence of different amounts of CH₃Cl in the feed (FIGS. 21-23); wherein T_{cat} is the temperature profile in the catalyst bed, and T_{fur} is the temperature of the furnace set point. FIG. 20 displays the temperature profile of the autothermal reactor without addition of CH₃Cl to the reactor. FIG. 21 displays the temperature profile of the autothermal reactor with addition of 10 ppm CH₃Cl to the reactor. FIG. 22 displays the temperature profile of the autothermal reactor with addition of 25 ppm CH₃Cl to the reactor. FIG. 23 displays the temperature profile of the autothermal reactor with addition of 50 ppm CH₃Cl to the reactor.

A comparison of the data in FIGS. 20-23 shows that at a concentration of CH₃Cl in the feed within the range of 10-25 ppm it is possible still keep the catalyst at ignition condition, while at 50 ppm CH₃Cl in the feed, the duration of stable performance of the autothermal reactor is reduced by comparison with lower concentrations of CH₃Cl in the feed.

Typically, the autothermal reactor operation comprised feeding the gaseous reactant mixture at a temperature of about 25° C., which was then heated in a furnace to a temperature in the range of about 550-650° C. for achieving the ignition of the catalyst. After catalyst ignition, the furnace temperature was reduced by turning off the power supplied to the furnace (which is shown in FIGS. 20-23 as the furnace temperature (T_{fur}). In the case when 25 ppm CH₃Cl was used in the feed, the furnace temperature (T_{fur}) decreased to 100° C. without dropping in catalyst bed temperature (T_{cat}).

### Example 18

Example 18 describes investigating OCM reactions with a supported Sr—La—W—MgO Catalyst 6 and with a pre-treated supported Sr—La—W—MgO Catalyst 7 (that has been prepared by pre-treating the supported Sr—La—W—MgO Catalyst 6 at room temperature for 8 h with 50 ppm CH₃Cl), in a quartz reactor with an ID of 2.3 mm with a catalyst loading of 20 mg. The supported Sr—La—W—MgO Catalyst 6 was prepared by mixing of nitrates salts of the component metals other than W, with addition of W₂O₅ oxide to the mixture of nitrates of catalyst components, followed by slow evaporation of the solution under stirring at 60-70° C. The flow rates of the gases were 34.8 cc/min of CH₄, 4.7 cc/min of O₂, and 0.5 cc/min of N₂. The methane feed in this Example 18 contained 0 ppm CH₃Cl or 50 ppm CH₃Cl. The results of Example 18 are displayed in FIG. 24.

FIG. 17 indicates that pre-treating the supported Sr—La—W—MgO Catalyst 6 at room temperature for 8 h with 50 ppm CH₃Cl results in pre-treated supported Sr—La—W—MgO Catalyst 7 having inferior properties over a temperature range of from about 550° C. to about 800° C., both in terms of C₂₊ selectivity and O₂ conversion. Given that pre-treating with 50 ppm CH₃Cl at room temperature leads to the decrease of O₂ conversion and C₂₊ selectivity, and with accounting for the data of Example 16, the positive effect of room temperature catalyst pre-treatment with CH₃Cl is dependent from on the catalyst composition.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

While various embodiments have been shown and described, modifications thereof can be made by one skilled in the art without departing from the spirit and teachings of the disclosure. The embodiments described herein are exemplary only, and are not intended to be limiting. Many variations and modifications of the subject matter disclosed herein are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R_{L} and an upper limit, R_{U} is disclosed, any number falling within the range is specifically disclosed. In particular, the following numbers within the range are specifically disclosed: R=R_{L}+k*(R_{U}−R_{L}), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the embodiments of the present disclosure. The discussion of a reference is not an admission that it is prior art to the present disclosure, especially any reference that may have a publication date after the priority date of this application. The disclosures of all patents, patent applications, and publications cited herein are hereby incorporated by reference, to the extent that they provide exemplary, procedural, or other details supplementary to those set forth herein.

### ADDITIONAL DISCLOSURE

The particular embodiments disclosed above are illustrative only, as the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present disclosure. Alternative embodiments that result from combining, integrating, and/or omitting features of the embodiment(s) are also within the scope of the disclosure. While compositions and methods are described in broader terms of “having”, “comprising,” “containing,” or “including” various components or steps, the compositions and methods can also “consist essentially of” or “consist of” the various components and steps. Use of the term “optionally” with respect to any element of a claim means that the element is required, or alternatively, the element is not required, both alternatives being within the scope of the claim.

Numbers and ranges disclosed above may vary by some amount. Whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent or other documents, the definitions that are consistent with this specification should be adopted.

Embodiments disclosed herein include:

A1: A process for producing ethylene, the process comprising: (a) introducing a reactant mixture to a reactor comprising an oxidative coupling of methane (OCM) catalyst composition, wherein the reactant mixture comprises methane (CH₄), oxygen (O₂), and a chlorine intermediate precursor, wherein the chlorine intermediate precursor is introduced into the reactor in an amount of from about 1 part per million (ppm) to about 500 ppm, from about 5 ppm to about 500 ppm, or from about 1 ppm to about 100 ppm, based on the total volume of the reactant mixture, and wherein the OCM catalyst composition is characterized by the general formula SrₐLa_{b}D_{c}E_{d}Oₓ; wherein D is a rare earth element; wherein E is another rare earth element; wherein D and E are not lanthanum and are not the same; wherein a is 1.0; wherein b is from about 0.3 to about 10.0; wherein c is from about 0.01 to about 10.0; wherein d is from about 0.01 to about 10.0; and wherein x balances the oxidation states; (b) allowing at least a portion of the reactant mixture to contact at least a portion of the OCM catalyst composition and react via an OCM reaction at an OCM reaction temperature to form a product mixture comprising olefins; and (c) recovering at least a portion of the olefins from the product mixture.

B1: A process for producing ethylene comprising: (a) continuously feeding a reactant mixture to a reactor to yield a product mixture, wherein the reactor comprises an oxidative coupling of methane (OCM) catalyst, wherein the reactant mixture comprises methane, oxygen, and a chlorine intermediate precursor, wherein the chlorine intermediate precursor is introduced into the reactor in an amount of from about 1 part per million (ppm) to about 500 ppm, from about 5 ppm to about 500 ppm, or from about 1 ppm to about 100 ppm, based on the total volume of the reactant mixture, wherein the product mixture comprises ethylene, ethane, and unreacted methane, wherein the OCM catalyst comprises strontium, a first rare earth element consisting of lanthanum, and second and third rare earth elements selected from the group consisting of scandium (Sc), cerium (Ce), praseodymium (Pr), neodymium (Nd), promethium (Pm), samarium (Sm), europium (Eu), gadolinium (Gd), yttrium (Y), terbium (Tb), dysprosium (Dy), holmium (Ho), erbium (Er), thulium (Tm), ytterbium (Yb), lutetium (Lu), and combinations thereof, wherein the second and the third rare earth elements are different; and (b) recovering at least a portion of the ethylene from the product mixture.

C1: A process for producing ethylene, the process comprising: (a) continuously feeding a reactant mixture to a reactor comprising an oxidative coupling of methane (OCM) catalyst composition until the reactor reaches an activation temperature, defined as the temperature at which the oxygen conversion reaches about 100%, and optionally holding at the activation temperature for an activation time to activate the OCM catalyst, wherein the reactant mixture comprises methane and oxygen, wherein the OCM catalyst composition comprises strontium, a first rare earth element consisting of lanthanum, and second and third rare earth elements selected from the group consisting of scandium (Sc), cerium (Ce), praseodymium (Pr), neodymium (Nd), promethium (Pm), samarium (Sm), europium (Eu), gadolinium (Gd), yttrium (Y), terbium (Tb), dysprosium (Dy), holmium (Ho), erbium (Er), thulium (Tm), ytterbium (Yb), lutetium (Lu), and combinations thereof, wherein the second and the third rare earth elements are different; (b) continuously feeding a reactant mixture to the reactor for a reaction time, whereby at least a portion of the reactant mixture contacts at least a portion of the OCM catalyst composition and reacts via an OCM reaction at an OCM reaction temperature to yield a product mixture comprising ethylene, wherein the reactant mixture comprises methane, oxygen, and a chlorine intermediate precursor, wherein the chlorine intermediate precursor is introduced into the reactor in an amount of from about 1 part per million (ppm) to about 500 ppm, from about 5 ppm to about 500 ppm, or from about 1 ppm to about 100 ppm, based on the total volume of the reactant mixture; and (c) recovering at least a portion of the ethylene from the product mixture.

Each of embodiments A1, B1, and C1 may have one or more of the following additional elements: Element i1: wherein the OCM reaction is characterized by an OCM catalyst ignition temperature, defined as the temperature at which the oxygen conversion is 50%, and wherein the process further comprises, prior to (a), introducing a reactant mixture comprising CH₄ and O₂ and excluding the chlorine intermediate precursor into the reactor until the reactor attains an activation temperature, defined as the temperature at which the oxygen conversion is about 100%, and maintaining the reactor at the activation temperature for an activation time period, wherein the activation temperature is above the OCM catalyst ignition temperature. Element i2: wherein (a) further comprises introducing the reactant mixture comprising the chlorine intermediate precursor to the reactor for a reaction time period, wherein the activation time period is from about 10 minutes to about 6 hours, and wherein the reaction time period is from about 1 day to about 2 days. Element i3: wherein the activation temperature is equal to or greater than the OCM reaction temperature. Element i4: wherein a difference between the activation temperature and the OCM reaction temperature is equal to or greater than about 25° C. Element i5: wherein the activation temperature is equal to or greater than about 800° C., and wherein the OCM reaction temperature is less than about 800° C. Element i6: wherein the OCM reaction temperature is above the OCM catalyst ignition temperature and below the activation temperature, and wherein the process further comprises reducing the temperature of the OCM reactor to the OCM reaction temperature prior to (a). Element i7: wherein the chlorine intermediate precursor is selected from the group consisting of hydrogen chloride, methyl chloride, methylene chloride, chloroform, carbon tetrachloride, ethyl chloride, 1,2-dichloroethane, trichloroethylene, and combinations thereof. Element i8: wherein the rare earth element D and the another rare earth element E are selected from the group consisting of scandium (Sc), cerium (Ce), praseodymium (Pr), neodymium (Nd), promethium (Pm), samarium (Sm), europium (Eu), gadolinium (Gd), yttrium (Y), terbium (Tb), dysprosium (Dy), holmium (Ho), erbium (Er), thulium (Tm), ytterbium (Yb), lutetium (Lu), and combinations thereof. Element i9: wherein the OCM catalyst composition further comprises a redox agent selected from the group consisting of manganese (Mn), tungsten (W), bismuth (Bi), antimony (Sb), tin (Sn), cerium (Ce), praseodymium (Pr), and combinations thereof. Element i10: wherein the redox agent is present in the OCM catalyst composition in an amount of from about 1 wt. % to about 25 wt. %, based on the total weight of the OCM catalyst composition; and wherein the redox agent is selected from the group consisting of manganese (Mn), tin (Sn), bismuth (Bi), cerium (Ce), and combinations thereof. Element i11: wherein the OCM catalyst composition comprises one or more oxides. Element i12; wherein the one or more oxides include one or more oxides of strontium; one or more oxides of lanthanum; one or more oxides of D; one or more oxides of E; or a combination thereof. Element i13: wherein the OCM catalyst composition has the general formula SrₐLa_{b}Yb_{c}E_{d}Oₓ, wherein a is 1.0; wherein b is from about 0.3 to about 10.0; wherein c is from about 0.01 to about 10.0; wherein d is from about 0.01 to about 10.0; and wherein x balances the oxidation states. Element i14: wherein the OCM catalyst composition has the general formula SrₐLa_{b}Yb_{c}Nd_{d}Oₓ; wherein a is 1.0; wherein b is from about 0.3 to about 10.0; wherein c is from about 0.01 to about 10.0; wherein d is from about 0.01 to about 10.0; and wherein x balances the oxidation states. Element i15: wherein b is 9, c is 1, and d is 7. Element i16: wherein the chlorine intermediate precursor is introduced continuously to the OCM reactor. Element i17: wherein the chlorine intermediate precursor is introduced discontinuously to the OCM reactor. Element i18: wherein the process is characterized by a selectivity to C₂ hydrocarbons that is increased when compared to a selectivity to C₂ hydrocarbons of an otherwise similar process conducted with a reactant mixture comprising methane and oxygen and excluding the chlorine intermediate precursor. Element i19: wherein the process is characterized by a methane conversion that is greater than or about equal to a methane conversion of an otherwise similar process conducted with a reactant mixture comprising methane and oxygen and excluding the chlorine intermediate precursor. Element i20: wherein the chlorine intermediate precursor is selected from the group consisting of hydrogen chloride, methyl chloride, methylene chloride, chloroform, carbon tetrachloride, ethyl chloride, 1,2-dichloroethane, trichloroethylene, and combinations thereof; and wherein the OCM catalyst is an unsupported catalyst having the general formula SrₐLa_{b}Yb_{c}Nd_{d}Oₓ; wherein a is 1.0; wherein b is from about 0.3 to about 10.0; wherein c is from about 0.01 to about 10.0; wherein d is from about 0.01 to about 10.0; and wherein x balances the oxidation states. Element i21: wherein the chlorine intermediate precursor is selected from the group consisting of hydrogen chloride, methyl chloride, methylene chloride, chloroform, carbon tetrachloride, ethyl chloride, 1,2-dichloroethane, trichloroethylene, and combinations thereof, wherein the reactant mixture in (a) excludes the chlorine intermediate precursor, and wherein the OCM catalyst is an unsupported catalyst having the general formula SrₐLa_{b}Yb_{c}Nd_{d}Oₓ; wherein a is 1.0; wherein b is from about 0.3 to about 10.0; wherein c is from about 0.01 to about 10.0; wherein d is from about 0.01 to about 10.0; and wherein x balances the oxidation states. Element i22: wherein the activation temperature is greater than the OCM reaction temperature, and wherein the process further comprises reducing the temperature to the OCM reaction temperature after the activation time.

A2: A process for producing ethylene, the process comprising: (a) introducing a reaction mixture to a reactor comprising an oxidative coupling of methane (OCM) catalyst composition, wherein the reaction mixture comprises methane (CH₄), oxygen (O₂), and a chlorine intermediate precursor, wherein the chlorine intermediate precursor is introduced into the reactor in an amount of from about 1 part per million (ppm) to about 500 ppm, from about 5 ppm to about 500 ppm, or from about 1 ppm to about 100 ppm, based on the total volume of the reaction mixture, and wherein the OCM catalyst composition is characterized by the general formula Laₐ—Ce_{b}—Oₓ; wherein a is 1.0; wherein b is from about 0.05 to about 1.0; and wherein x balances the oxidation states; (b) allowing at least a portion of the reaction mixture to contact at least a portion of the OCM catalyst composition and react via an OCM reaction at an OCM reaction temperature to form a product mixture comprising olefins; and (c) recovering at least a portion of the olefins from the product mixture.

B2: A process for producing ethylene comprising: (a) continuously feeding a reaction mixture to a reactor to yield a product mixture, wherein the reactor comprises an oxidative coupling of methane (OCM) catalyst, wherein the reaction mixture comprises methane, oxygen, and a chlorine intermediate precursor, wherein the chlorine intermediate precursor is introduced into the reactor in an amount of from about 1 part per million (ppm) to about 500 ppm, from about 5 to about 500 ppm, from about 5 ppm to about 50 ppm, or less than or equal to about 50, 40, 35, 30, 25, 20, or 15 ppm, based on the total volume of the reaction mixture, wherein the product mixture comprises ethylene, ethane, and unreacted methane, wherein the OCM catalyst is characterized by the general formula Laₐ—Ce_{b}—Oₓ; wherein a is 1.0; wherein b is from about 0.05 to about 1.0; and wherein x balances the oxidation states; and (b) recovering at least a portion of the ethylene from the product mixture.

C2: A process for producing ethylene, the process comprising: (a) continuously feeding a reaction mixture to a reactor comprising an oxidative coupling of methane (OCM) catalyst composition until the reactor reaches an activation temperature, defined as the temperature at which the oxygen conversion reaches about 100%, and optionally holding at the activation temperature for an activation time to activate the OCM catalyst, wherein the reaction mixture comprises methane and oxygen, wherein the OCM catalyst composition is characterized by the general formula Laₐ—Ce_{b}—Oₓ; wherein a is 1.0; wherein b is from about 0.05 to about 1.0; and wherein x balances the oxidation states; (b) continuously feeding a reaction mixture to the reactor for a reaction time, whereby at least a portion of the reaction mixture contacts at least a portion of the OCM catalyst composition and reacts via an OCM reaction at an OCM reaction temperature to yield a product mixture comprising ethylene, wherein the reaction mixture comprises methane, oxygen, and a chlorine intermediate precursor, wherein the chlorine intermediate precursor is introduced into the reactor in an amount of from about 1 part per million (ppm) to about 500 ppm, from about 5 to about 500 ppm, from about 5 ppm to about 50 ppm, or less than or equal to about 50, 40, 35, 30, 25, 20, or 15 ppm, 1 part per million (ppm) to about 500 ppm, from about 5 ppm to about 500 ppm, or from about 1 ppm to about 100 ppm, based on the total volume of the reaction mixture; and (c) recovering at least a portion of the ethylene from the product mixture.

Each of embodiments A2, B2, and C2 may have one or more of the following additional elements: Element ii1: wherein the OCM reaction is characterized by an OCM catalyst ignition temperature, defined as the temperature at which the oxygen conversion is 50%, and wherein the process further comprises, prior to (a), introducing a reaction mixture comprising CH₄ and O₂ and excluding the chlorine intermediate precursor into the reactor until the reactor attains an activation temperature, defined as the temperature at which the oxygen conversion is about 100%, and maintaining the reactor at the activation temperature for an activation time period, wherein the activation temperature is above the OCM catalyst ignition temperature. Element ii2: wherein (a) further comprises introducing the reaction mixture comprising the chlorine intermediate precursor to the reactor for a reaction time period, wherein the activation time period is from about 10 minutes to about 6 hours, and wherein the reaction time period is from about 1 day to about 2 days. Element ii3: wherein the activation temperature is equal to or greater than the OCM reaction temperature. Element ii4: wherein a difference between the activation temperature and the OCM reaction temperature is equal to or greater than about 25° C. Element ii5: wherein the activation temperature is equal to or greater than about 800° C., and wherein the OCM reaction temperature is less than about 800° C. Element ii6: wherein the OCM reaction temperature is above the OCM catalyst ignition temperature and below the activation temperature, and wherein the process further comprises reducing the temperature of the OCM reactor to the OCM reaction temperature prior to (a). Element ii7: wherein the chlorine intermediate precursor comprises hydrogen chloride, methyl chloride, methylene chloride, chloroform, carbon tetrachloride, ethyl chloride, 1,2-dichloroethane, trichloroethylene, or a combination thereof. Element ii8: wherein b is from about 0.0625 to about 1. Element ii9: wherein the OCM catalyst composition comprises one or more oxides. Element ii10: wherein the one or more oxides include one or more oxides of lanthanum; one or more oxides of cerium; or a combination thereof. Element ii11: wherein the chlorine intermediate precursor is introduced continuously to the OCM reactor. Element ii12: wherein the chlorine intermediate precursor is introduced discontinuously to the OCM reactor. Element ii13: wherein the process is characterized by a selectivity to C₂ hydrocarbons that is increased when compared to a selectivity to C₂ hydrocarbons of an otherwise similar process conducted with a reaction mixture comprising methane and oxygen and excluding the chlorine intermediate precursor. Element ii14: wherein (i) the process is characterized by a methane conversion that is greater than or about equal to a methane conversion of an otherwise similar process conducted with a reaction mixture comprising methane and oxygen and excluding the chlorine intermediate precursor; (ii) the process is characterized by a selectivity to one or more deep oxidation products that is greater than or about equal to a selectivity to the one or more deep oxidation products of an otherwise similar process conducted with a reaction mixture comprising methane and oxygen and excluding the chlorine intermediate precursor; or (iii) both (i) and (ii). Element ii15: wherein the chlorine intermediate precursor comprises hydrogen chloride, methyl chloride, methylene chloride, chloroform, carbon tetrachloride, ethyl chloride, 1,2-dichloroethane, trichloroethylene, or a combination thereof. Element ii16: wherein the reaction mixture in (a) excludes the chlorine intermediate precursor. Element ii 17: wherein the activation temperature is greater than the OCM reaction temperature, and wherein the process further comprises reducing the temperature to the OCM reaction temperature after the activation time.

A first aspect, which is a method for producing C₂₊ hydrocarbons comprising (a) introducing a reactant mixture to an oxidative coupling of methane (OCM) reactor comprising a supported OCM catalyst composition; wherein the reactant mixture comprises methane (CH₄), oxygen (O₂), and a chlorine intermediate precursor; wherein the chlorine intermediate precursor is present in the reactant mixture in an amount of from about 1 part per million (ppm) to about 50 ppm; wherein the supported OCM catalyst composition is characterized by the general formula Mn—Na₂WO₄/SiO₂; wherein the supported OCM catalyst composition optionally comprises a metal oxide characterized by the general formula MOₓ; wherein M is a metal with redox properties; and wherein x balances the oxidation states; (b) allowing at least a portion of the reactant mixture to contact at least a portion of the supported OCM catalyst composition and react via an OCM reaction to form a product mixture comprising unreacted methane and C₂₊ hydrocarbons; (c) recovering at least a portion of the product mixture from the OCM reactor; and (d) recovering at least a portion of the C₂₊ hydrocarbons from the product mixture.

A second aspect, which is the method of the first aspect, wherein the chlorine intermediate precursor is selected from the group consisting of hydrogen chloride, methyl chloride, methylene chloride, chloroform, carbon tetrachloride, chloroethane (ethyl chloride), 1,1-dichloroethane, 1,2-dichloroethane, vinyl chloride, dichloroethene, 1,1-dichloroethylene (vinylidene chloride), cis-1,2-dichloroethylene, 1,2-trans-dichloro ethylene, trichloroethylene (TCE), 1,1,1-trichloroethane, 1,1,2-trichloroethane (vinyl trichloride), 1,1,1-trichloroethene, 1,1,1,2-tetrachloroethane, 1,1,2,2-tetrachloroethane (acetylene tetrachloride), tetrachloroethylene (perchloroethylene; PCE), pentachloroethane, hexachloroethane, chloropropane, 1,2-dichloropropane (propylene dichloride), 1-chloro-2-propene, 1,3-cis-dichloro-1-propene, 1,3-trans-dichloropropene, trichloropropane, 1,2,3-trichloropropane, chloroprene, 2-butylene dichloride, hexachlorobutadiene, hexachlorocyclopentadiene, monochlorocyclohexane, monochlorobenzene, and combinations thereof.

A third aspect, which is the method of any one of the first and the second aspects, wherein a weight ratio of MOₓ to Mn—Na₂WO₄/SiO₂ is from about 0.01:1 to about 0.2:1.

A fourth aspect, which is the method of any one of the first through the third aspects, wherein the supported OCM catalyst composition comprises the metal M in an amount of from about 0.1 wt. % to about 10 wt. %, based on the total weight of the supported OCM catalyst composition.

A fifth aspect, which is the method of any one of the first through the fourth aspects, wherein the supported OCM catalyst composition comprises manganese (Mn) in an amount of from about 0.1 wt. % to about 10 wt. %, based on the total weight of the supported OCM catalyst composition.

A sixth aspect, which is the method of any one of the first through the fifth aspects, wherein the supported OCM catalyst composition comprises Na₂WO₄ in an amount of from about 0.1 wt. % to about 15 wt. %, based on the total weight of the supported OCM catalyst composition.

A seventh aspect, which is the method of any one of the first through the sixth aspects, wherein the metal M is selected from the group consisting of tin (Sn), antimony (Sb), bismuth (Bi), iron (Fe), chromium (Cr), molybdenum (Mo), tungsten (W), vanadium (V), tantalum (Ta), niobium (Nb), gallium (Ga), rhenium (Re), lead (Pb), cobalt (Co), nickel (Ni), copper (Cu), and combinations thereof.

An eighth aspect, which is the method of any one of the first through the seventh aspects, wherein MOₓ comprise a single metal oxide, mixtures of single metal oxides, a mixed metal oxide, mixtures of mixed metal oxides, mixtures of single metal oxides and mixed metal oxides, or combinations thereof.

A ninth aspect, which is the method of any one of the first through the eighth aspects, wherein MOₓ comprise nanostructures, wherein a nanostructure is defined as a three-dimensional object characterized by at least one external dimension of less than about 1,000 nm.

A tenth aspect, which is the method of any one of the first through the ninth aspects, wherein the supported OCM catalyst composition is characterized by the general formula (SbOₓ)—Mn—Na₂WO₄/SiO₂.

An eleventh aspect, which is the method of any one of the first through the tenth aspects, wherein the OCM reactor is characterized by a reactor effluent temperature of from about 700° C. to about 925° C.

A twelfth aspect, which is the method of any one of the first through the eleventh aspects, wherein the reactant mixture is characterized by a CH₄/O₂ molar ratio of from about 4:1 to about 10:1.

A thirteenth aspect, which is the method of any one of the first through the twelfth aspects, wherein the OCM reactor is characterized by a residence time of from about 10 milliseconds (ms) to about 2 seconds (s).

A fourteenth aspect, which is the method of any one of the first through the thirteenth aspects further comprising, prior to the step (a), calcining the supported OCM catalyst composition at a temperature of from about 700° C. to about 1,000° C.

A fifteenth aspect, which is the method of any one of the first through the fourteenth aspects, wherein the supported OCM catalyst composition in the presence of the chlorine intermediate precursor is characterized by an O₂ conversion that is increased by equal to or greater than about 10% when compared to an O₂ conversion of an otherwise similar supported OCM catalyst composition in the absence of the chlorine intermediate precursor.

A sixteenth aspect, which is the method of any one of the first through the fifteenth aspects, wherein the supported OCM catalyst composition in the presence of the chlorine intermediate precursor is characterized by a catalyst activity variation within about ±10% of a target catalyst activity over a time period of equal to or greater than about 50 hours (h), wherein the catalyst activity is defined as the O₂ conversion under a set of given OCM reactor operational parameters, and wherein the target catalyst activity is defined as a target O₂ conversion of equal to or greater than about 90% under the same set of given OCM reactor operational parameters.

A seventeenth aspect, which is a method for producing C₂₊ hydrocarbons comprising (a) introducing a reactant mixture to an oxidative coupling of methane (OCM) reactor comprising a supported OCM catalyst composition; wherein the reactant mixture comprises methane (CH₄), oxygen (O₂), and a chlorine intermediate precursor; wherein the chlorine intermediate precursor is present in the reactant mixture in an amount of from about 4 parts per million (ppm) to about 25 ppm; wherein the supported OCM catalyst composition is characterized by the general formula (MOₓ)—Mn—Na₂WO₄/SiO₂; wherein M is a metal with redox properties; wherein x balances the oxidation states; wherein the OCM reactor is characterized by a reactor effluent temperature of from about 800° C. to about 875° C.; wherein the OCM reactor is characterized by a residence time of from about 500 milliseconds (ms) to about 1.5 seconds (s); and wherein the reactant mixture is characterized by a CH₄/O₂ molar ratio of from about 5:1 to about 8:1; (b) allowing at least a portion of the reactant mixture to contact at least a portion of the supported OCM catalyst composition and react via an OCM reaction to form a product mixture comprising unreacted methane and C₂₊ hydrocarbons; (c) recovering at least a portion of the product mixture from the OCM reactor; and (d) recovering at least a portion of the C₂₊ hydrocarbons from the product mixture.

An eighteenth aspect, which is the method of the seventeenth aspect, wherein the metal M is selected from the group consisting of tin (Sn), antimony (Sb), lead (Pb), and combinations thereof.

A nineteenth aspect, which is the method of any one of the seventeenth and the eighteenth aspects, wherein the supported OCM catalyst composition comprises the metal M in an amount of from about 1 wt. % to about 5 wt. %, based on the total weight of the supported OCM catalyst composition; wherein the supported OCM catalyst composition comprises manganese (Mn) in an amount of from about 3 wt. % to about 8 wt. %, based on the total weight of the supported OCM catalyst composition; and wherein the supported OCM catalyst composition comprises Na₂WO₄ in an amount of from about 1 wt. % to about 10 wt. %, based on the total weight of the supported OCM catalyst composition.

A twentieth aspect, which is the method of any one of the seventeenth through the nineteenth aspects, wherein the supported OCM catalyst composition in the presence of the chlorine intermediate precursor is characterized by an O₂ conversion that is increased by equal to or greater than about 15% when compared to an O₂ conversion of an otherwise similar supported OCM catalyst composition in the absence of the chlorine intermediate precursor; and wherein the supported OCM catalyst composition in the presence of the chlorine intermediate precursor is characterized by a catalyst activity variation within about ±5% of a target catalyst activity over a time period of equal to or greater than about 50 hours (h), wherein the catalyst activity is defined as the O₂ conversion under a set of given OCM reactor operational parameters, and wherein the target catalyst activity is defined as a target O₂ conversion of equal to or greater than about 90% under the same set of given OCM reactor operational parameters.

A twenty-first aspect, which is a process comprising oxidative conversion of methane (OCM) in the presence of mixed oxide catalysts in an auto-thermal reactor with a very short contact time of about 1-100 milliseconds and with using a methane-oxygen mixture with the addition small quantities of chlorine containing ingredients to the feed; and/or pretreatment of the catalysts with the relatively low levels (0.1-100 ppm) of a chlorine intermediate precursor, leading to the efficient management of the reaction heat and improvement of the selectivity to C₂₊ hydrocarbons.

A twenty-second aspect, which is the process of the twenty-first aspect, wherein the applied reactor works in an auto-thermal mode and the reaction is carried out with the heat produced from the reaction used to preheat the incoming feed in situ without the use of an external heat exchange device.

A twenty-third aspect, which is the process of any one of the twenty-first and the twenty-second aspects, wherein the reactor is operated under autothermal conditions, and wherein a catalyst bed is originally heated by the high flow of methane or an inert gas such as nitrogen to the catalyst ignition temperature.

A twenty-fourth aspect, which is the process of any one of the twenty-first through the twenty-third aspects, wherein the reactor is operated under autothermal conditions, and wherein an ignition temperature of the catalyst varies between about 450° C. to about 650° C.

A twenty-fifth aspect, which is the process of any one of the twenty-first through the twenty-fourth aspects, wherein the reactor is operated under autothermal conditions, wherein the catalyst at ignition temperature is ignited with the CH₄+O₂ feed and after ignition of the catalyst, and wherein the catalyst bed temperature is maintained between about 750° C. to about 950° C. by allowing the CH₄/O₂ molar ratio in the feed to ramp down along with the furnace heat output.

A twenty-sixth aspect, which is the process of any one of the twenty-first through the twenty-fifth aspects, wherein the catalyst bed temperature is controlled through variation of the CH₄/O₂ molar ratio in the presence or absence of an external source of heat.

A twenty-seventh aspect, which is the process of any one of the twenty-first through the twenty-sixth aspects, wherein the CH₄/O₂ molar ratio decreases from about 16 to about 4.

A twenty-eighth aspect, which is the process of any one of the twenty-first through the twenty-seventh aspects, wherein the catalyst bed temperature is maintained between about 800° C. to about 900° C. to provide for an increase in C₂₊ selectivity.

A twenty-ninth aspect, which is the process of any one of the twenty-first through the twenty-eighth aspects, wherein the catalyst used in auto-thermal reactor is a mixture of oxides of one or more alkali earth metals and one or more rare earth elements.

A thirtieth aspect, which is the process of any one of the twenty-first through the twenty-ninth aspects, wherein the catalyst used in auto-thermal reactor is a mixture of one or more oxides of Sr, one or more oxides of Mg, and one or more oxides of La.

A thirty-first aspect, which is the process of any one of the twenty-first through the thirtieth aspects, wherein the catalyst used in auto-thermal reactor is characterized by the general formula Sr_{1.0}La_{0.18}Mg_{0.13}Oₓ, wherein x balances the oxidation states.

A thirty-second aspect, which is the process of any one of the twenty-first through the thirty-first aspects, wherein the OCM reaction is carried out under isothermal conditions with a variation of reaction temperature between about 650° C. to about 900° C.

A thirty-third aspect, which is the process of any one of the twenty-first through the thirty-second aspects, wherein the OCM reaction is carried out under isothermal conditions with a CH₄/O₂ molar ratio in the feed composition of about 7.4.

A thirty-fourth aspect, which is the process of any one of the twenty-first through the thirty-third aspects, wherein the OCM reaction is carried out under isothermal conditions with a CH₄/O₂ molar ratio in the feed composition of about 7.4 and with the addition to the feed of a chlorine intermediate precursor in an amount of from about 1 ppm to about 100 ppm, based on the total volume of the feed.

A thirty-fifth aspect, which is the process of any one of the twenty-first through the thirty-third aspects, wherein the OCM reaction is carried out under isothermal conditions with a CH₄/O₂ molar ratio in the feed composition of about 7.4 and with the addition to the feed of a chlorine intermediate precursor in an amount of from about 10 ppm to about 50 ppm, based on the total volume of the feed.

A thirty-sixth aspect, which is the process of any one of the twenty-first through the thirty-fifth aspects, wherein the chlorine intermediate precursor is selected from the group consisting of CH₃Cl, C₂H₅Cl, C₂H₄Cl₂, HCl, Cl₂, and combinations thereof.

A thirty-seventh aspect, which is the process of any one of the twenty-first through the thirty-sixth aspects, wherein the chlorine intermediate precursor is CH₃Cl.

A thirty-eighth aspect, which is the process of any one of the twenty-first through the thirty-seventh aspects, wherein the catalyst is pretreated with a mixture containing methane and 50 ppm CH₃Cl, based on the total volume of the mixture.

A thirty-ninth aspect, which is the process of any one of the twenty-first through the thirty-eighth aspects, wherein the OCM reaction is carried out under isothermal conditions, and wherein the catalyst is pretreated with a mixture containing methane and 50 ppm CH₃Cl, based on the total volume of the mixture.

A fortieth aspect, which is the process of any one of the twenty-first through the thirty-ninth aspects, wherein the OCM reaction is carried out under autothermal conditions, and wherein the catalyst is pretreated with a mixture containing methane and 50 ppm CH₃Cl, based on the total volume of the mixture.

A forty-first aspect, which is the process of any one of the twenty-first through the fortieth aspects, wherein the catalyst has a pre-contact and/or pre-treatment with the chlorine intermediate precursor C₂₊ selectivity of equal to or greater than about 60%.

A forty-second aspect, which is the process of any one of the twenty-first through the forty-first aspects, wherein the catalyst has a pre-contact and/or pre-treatment with the chlorine intermediate precursor C₂₊ selectivity of equal to or greater than about 70%.

A forty-third aspect, which is the process of any one of the twenty-first through the forty-second aspects, wherein the catalyst has a pre-contact and/or pre-treatment with the chlorine intermediate precursor C₂₊ selectivity of from about 60% to about 80%.

A forty-fourth aspect, which is the process of any one of the twenty-first through the forty-third aspects, wherein addition of from about 20 ppm to about 50 ppm chlorine intermediate precursor to the CH₄+O₂ feed, based on the total volume of the feed, under isothermal conditions leads to an increase C₂₊ selectivity of from about 4% to about 6%.

A forty-fifth aspect, which is the process of any one of the twenty-first through the forty-fourth aspects, wherein addition of from about 20 ppm to about 50 ppm chlorine intermediate precursor to the CH₄+O₂ feed, based on the total volume of the feed, under autothermal conditions leads to an increase C₂₊ selectivity of from about 4% to about 6%.

A forty-sixth aspect, which is a process for producing C₂₊ hydrocarbons comprising (a) introducing a reactant mixture to an oxidative coupling of methane (OCM) reactor comprising an OCM catalyst composition; wherein the reactant mixture comprises methane (CH₄), oxygen (O₂), and a chlorine intermediate precursor; wherein the chlorine intermediate precursor is present in the reactant mixture in an amount of from about 1 part per million (ppm) to about 100 ppm, based on the total volume of the reactant mixture; (b) allowing at least a portion of the reactant mixture to contact at least a portion of the OCM catalyst composition and react via an OCM reaction to form a product mixture comprising unreacted methane and C₂₊ hydrocarbons; (c) recovering at least a portion of the product mixture from the OCM reactor; and (d) recovering at least a portion of the C₂₊ hydrocarbons from the product mixture.

A forty-seventh aspect, which is the process of the forty-sixth aspect, wherein the process for producing C₂₊ hydrocarbons is characterized by improved performance when compared to an otherwise similar process that employs a reactant mixture comprising CH₄ and O₂, without the chlorine intermediate precursor; wherein the improved performance of the process is defined by the process having at least one modification selected from the group consisting of increased C₂₊ selectivity, decreased selectivity to carbon dioxide (CO₂), increased catalyst activity, increased methane conversion, increased catalyst stability, increased catalyst life-time, decreased reaction temperature effective for achieving an O₂ conversion of equal to or greater than about 90%, and combinations thereof; wherein the catalyst activity is defined as the O₂ conversion under a set of given OCM reactor operational parameters; wherein the catalyst stability is defined as a catalyst activity variation within about ±10% of a target catalyst activity over a time period of equal to or greater than about 50 hours (h); and wherein the target catalyst activity is defined as a target O₂ conversion of equal to or greater than about 90% under the same set of given OCM reactor operational parameters.

A forty-eighth aspect, which is the process of any one of the forty-sixth and the forty-seventh aspects, wherein the chlorine intermediate precursor is selected from the group consisting of hydrogen chloride, methyl chloride, methylene chloride, chloroform, carbon tetrachloride, chloroethane (ethyl chloride), 1,1-dichloroethane, 1,2-dichloroethane, vinyl chloride, dichloroethene, 1,1-dichloroethylene (vinylidene chloride), cis-1,2-dichloroethylene, 1,2-trans-dichloroethylene, trichloroethylene (TCE), 1,1,1-trichloroethane, 1,1,2-trichloroethane (vinyl trichloride), 1,1,1-trichloroethene, 1,1,1,2-tetrachloroethane, 1,1,2,2-tetrachloroethane (acetylene tetrachloride), tetrachloroethylene (perchloroethylene; PCE), pentachloroethane, hexachloroethane, chloropropane, 1,2-dichloropropane (propylene dichloride), 1-chloro-2-propene, 1,3-cis-dichloro-1-propene, 1,3-trans-dichloropropene, trichloropropane, 1,2,3-trichloropropane, chloroprene, 2-butylene dichloride, hexachlorobutadiene, hexachlorocyclopentadiene, monochlorocyclohexane, monochlorobenzene, and combinations thereof.

A forty-ninth aspect, which is the process of any one of the forty-sixth through the forty-eighth aspects, wherein the OCM catalyst composition comprises a single metal oxide, mixtures of single metal oxides, a mixed metal oxide, mixtures of mixed metal oxides, or combinations thereof; and wherein the OCM catalyst composition is a supported OCM catalyst composition and/or a unsupported OCM catalyst composition.

A fiftieth aspect, which is the process of the forty-ninth aspect, wherein the OCM catalyst composition is a supported OCM catalyst composition comprising a support, wherein at least a portion of the OCM catalyst composition contacts, coats, is embedded in, is supported by, and/or is distributed throughout at least a portion of the support; and wherein the support comprises MgO, Al₂O₃, SiO₂, ZrO₂, TiO₂, or combinations thereof.

A fifty-first aspect, which is the process of any one of the forty-ninth and the fiftieth aspects, wherein the OCM catalyst composition is characterized by the general formula Aₐ₁Z_{b1}E_{c1}D_{d1}Oₓ₁; wherein A is an alkaline earth metal; wherein Z is a first rare earth element; wherein E is a second rare earth element; wherein D is a redox agent or a third rare earth element; wherein the first rare earth element, the second rare earth element, and the third rare earth element, when present, are not the same; wherein a1 is 1.0; wherein b1 is from about 0.1 to about 10.0; wherein c1 is from about 0 to about 10.0; and wherein d1 is from about 0 to about 10.0; and wherein x1 balances the oxidation states.

A fifty-second aspect, which is the process of the fifty-first aspect, wherein the alkaline earth metal is selected from the group consisting of magnesium (Mg), calcium (Ca), strontium (Sr), barium (Ba), and combinations thereof; wherein the first rare earth element, the second rare earth element, and the third rare earth element can each independently be selected from the group consisting of lanthanum (La), scandium (Sc), cerium (Ce), praseodymium (Pr), neodymium (Nd), promethium (Pm), samarium (Sm), europium (Eu), gadolinium (Gd), yttrium (Y), terbium (Tb), dysprosium (Dy), holmium (Ho), erbium (Er), thulium (Tm), ytterbium (Yb), lutetium (Lu), and combinations thereof; and wherein the redox agent is selected from the group consisting of manganese (Mn), tungsten (W), bismuth (Bi), antimony (Sb), tin (Sn), cerium (Ce), praseodymium (Pr), and combinations thereof.

A fifty-third aspect, which is the process of any one of the fifty-first and the fifty-second aspects, wherein the OCM catalyst composition has the general formula Srₐ₁La_{b1}E_{c1}D_{d1}Oₓ₁; wherein E is a second rare earth element other than lanthanum (La); wherein D is a third rare earth element other than lanthanum (La); wherein the second rare earth element and the third rare earth element are different; wherein a1 is 1.0; wherein b1 is from about 0.1 to about 10.0; wherein c1 is from about 0.01 to about 10.0; wherein d1 is from about 0.01 to about 10.0; and wherein x1 balances the oxidation states.

A fifty-fourth aspect, which is the process of the fifty-third aspect, wherein the OCM catalyst composition has the general formula Srₐ₁La_{b1}Yb_{c1}Nd_{d1}Oₓ₁; wherein a1 is 1.0; wherein b1 is from about 0.1 to about 10.0; wherein c1 is from about 0.01 to about 10.0; wherein d1 is from about 0.01 to about 10.0; and wherein x1 balances the oxidation states.

A fifty-fifth aspect, which is the process of the fifty-fourth aspect, wherein the OCM catalyst composition comprises a unsupported OCM catalyst composition and/or a supported OCM catalyst composition; and wherein, when the OCM catalyst composition comprises a supported OCM catalyst composition, the supported OCM catalyst composition comprises an alumina support.

A fifty-sixth aspect, which is the process of any one of the fifty-first and the fifty-second aspects, wherein the OCM catalyst composition is a supported OCM catalyst composition comprising a MgO support; wherein the OCM catalyst composition has the general formula Srₐ₁La_{b1}Oₓ₁/MgO; wherein a1 is 1.0; wherein b1 is from about 0.1 to about 10.0; and wherein x1 balances the oxidation states.

A fifty-seventh aspect, which is the process of any one of the forty-sixth through the forty-ninth aspects, wherein the OCM catalyst composition is characterized by the general formula Laₐ₂Ce_{b2}Oₓ₂; wherein a2 is 1.0; wherein b2 is from about 0.3 to about 10.0; and wherein x2 balances the oxidation states.

A fifty-eighth aspect, which is the process of any one of the forty-sixth through the forty-ninth aspects, wherein the OCM catalyst composition is a supported OCM catalyst composition comprising a silica support; wherein the OCM catalyst composition is characterized by the general formula Mn—Na₂WO₄/SiO₂; wherein the supported OCM catalyst composition optionally comprises a metal oxide characterized by the general formula MOₓ₃; wherein M is a metal with redox properties; and wherein x3 balances the oxidation states.

A fifty-ninth aspect, which is the process of the fifty-eighth aspect, wherein the metal M is selected from the group consisting of tin (Sn), antimony (Sb), bismuth (Bi), iron (Fe), chromium (Cr), molybdenum (Mo), tungsten (W), vanadium (V), tantalum (Ta), niobium (Nb), gallium (Ga), rhenium (Re), lead (Pb), cobalt (Co), nickel (Ni), copper (Cu), and combinations thereof; and wherein the supported OCM catalyst composition comprises the metal M in an amount of from about 0.1 wt. % to about 10 wt. %, based on the total weight of the supported OCM catalyst composition.

A sixtieth aspect, which is the process of the fifty-eighth aspect, wherein the OCM catalyst composition has the general formula (SbOₓ₃)—Mn—Na₂WO₄/SiO₂.

A sixty-first aspect, which is the process of any one of the forty-sixth through the fifty-ninth aspects, wherein the OCM reactor is operated under isothermal conditions.

A sixty-second aspect, which is the process of any one of the forty-sixth through the fifty-ninth aspects, wherein the OCM reactor is operated under autothermal conditions.

A sixty-third aspect, which is the process of the sixty-second aspect, wherein the OCM reactor is characterized by a feed temperature of from about 25° C. to about 400° C., thereby providing for igniting the OCM catalyst composition at an ignition temperature of from about 450° C. to about 650° C.

A sixty-fourth aspect, which is the process of any one of the sixty-second and the sixty-third aspects, wherein the autothermal conditions are maintained by decreasing a CH₄/O₂ molar ratio in the reactant mixture while and/or subsequent to removing an external heat supply to the OCM catalyst composition.

A sixty-fifth aspect, which is the process of the sixty-fourth aspect, wherein the CH₄/O₂ molar ratio in the reactant mixture is decreased from about 16 to about 4.

A sixty-sixth aspect, which is the process of any one of the sixty-second through the sixty-fifth aspects, wherein the OCM reactor is characterized by a catalyst bed temperature maintained between about 700° C. to about 1,000° C. while and/or subsequent to removing an external heat supply to the OCM catalyst composition.

A sixty-seventh aspect, which is a process for producing ethylene comprising (a) introducing a reactant mixture to an oxidative coupling of methane (OCM) reactor comprising an OCM catalyst composition; wherein the reactant mixture comprises methane (CH₄), oxygen (O₂), and methyl chloride (CH₃Cl); wherein CH₃Cl is present in the reactant mixture in an amount of from about 1 part per million (ppm) to about 50 ppm, based on the total volume of the reactant mixture; (b) allowing at least a portion of the reactant mixture to contact at least a portion of the OCM catalyst composition and react via an OCM reaction to form a product mixture comprising unreacted methane and C₂₊ olefins; (c) recovering at least a portion of the product mixture from the OCM reactor; and (d) recovering at least a portion of ethylene from the product mixture.

A sixty-eighth aspect, which is the process of the sixty-seventh aspect, wherein the process for producing ethylene is characterized by improved performance when compared to an otherwise similar process that employs a reactant mixture comprising CH₄ and O₂, without the CH₃Cl; wherein the improved performance of the process is defined by the process having a C₂₊ selectivity that is increased by equal to or greater than about 2% when compared to a C₂₊ selectivity of an otherwise similar process that employs a reactant mixture comprising CH₄ and O₂, without the CH₃Cl.

A sixty-ninth aspect, which is the process of any one of the sixty-seventh and the sixty-eighth aspects, wherein the process for producing ethylene is characterized by improved performance when compared to an otherwise similar process that employs a reactant mixture comprising CH₄ and O₂, without the CH₃Cl; wherein the improved performance of the process is defined by the process having (i) an O₂ conversion under a set of given OCM reactor operational parameters that is increased by equal to or greater than about 5% when compared to an O₂ conversion under the same set of given OCM reactor operational parameters of an otherwise similar process that employs a reactant mixture comprising CH₄ and O₂, without the CH₃Cl; and/or (ii) a catalyst activity variation within about ±5% of a target catalyst activity over a time period of equal to or greater than about 50 hours (h), wherein the catalyst activity is defined as the O₂ conversion under a set of given OCM reactor operational parameters, and wherein the target catalyst activity is defined as a target O₂ conversion of equal to or greater than about 90% under the same set of given OCM reactor operational parameters.

A seventieth aspect, which is the process of any one of the sixty-seventh through the sixty-ninth aspects, wherein the OCM catalyst composition is characterized by the general formula Srₐ₁La_{b1}Yb_{c1}Nd_{d1}Oₓ₁; wherein a1 is 1.0; wherein b1 is from about 0.3 to about 10.0; wherein c1 is from about 0.05 to about 10.0; wherein d1 is from about 0.05 to about 10.0; and wherein x1 balances the oxidation states.

A seventy-first aspect, which is the process of any one of the sixty-seventh through the seventieth aspects, wherein the CH₃Cl is present in the reactant mixture in an amount of from about 20 ppm to about 50 ppm, based on the total volume of the reactant mixture.

A seventy-second aspect, which is the process of any one of the sixty-seventh through the seventy-first aspects, wherein the OCM reactor is operated under autothermal conditions; and wherein, prior to step (b) of allowing at least a portion of the reactant mixture to contact at least a portion of the OCM catalyst composition and react via an OCM reaction, the OCM catalyst composition is pre-treated by contacting at least a portion of the OCM catalyst composition with a pre-treating mixture comprising CH₄ and from about 25 ppm to about 75 ppm CH₃Cl for a time period of from about 2 hours to about 12 hours.

While preferred embodiments of the invention have been shown and described, modifications thereof can be made by one skilled in the art without departing from the teachings of this disclosure. The embodiments described herein are exemplary only, and are not intended to be limiting. Many variations and modifications of the invention disclosed herein are possible and are within the scope of the invention.

Numerous other modifications, equivalents, and alternatives, will become apparent to those skilled in the art once the above disclosure is fully appreciated. It is intended that the following claims be interpreted to embrace all such modifications, equivalents, and alternatives where applicable. Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

What is claimed is:

1. A process for producing C₂₊ hydrocarbons comprising:
(a) introducing a reactant mixture to an oxidative coupling of methane (OCM) reactor comprising an OCM catalyst composition; wherein the reactant mixture comprises methane (CH₄), oxygen (O₂), and a chlorine intermediate precursor; wherein the chlorine intermediate precursor is present in the reactant mixture in an amount of from about 1 part per million (ppm) to about 100 ppm, based on the total volume of the reactant mixture;
(b) allowing at least a portion of the reactant mixture to contact at least a portion of the OCM catalyst composition and react via an OCM reaction to form a product mixture comprising unreacted methane and C₂₊ hydrocarbons;
(c) recovering at least a portion of the product mixture from the OCM reactor; and
(d) recovering at least a portion of the C₂₊ hydrocarbons from the product mixture.

2. The process of claim 1, wherein the chlorine intermediate precursor is selected from the group consisting of hydrogen chloride, methyl chloride, methylene chloride, chloroform, carbon tetrachloride, chloroethane (ethyl chloride), 1,1-dichloroethane, 1,2-dichloroethane, vinyl chloride, dichloroethene, 1,1-dichloroethylene (vinylidene chloride), cis-1,2-dichloroethylene, 1,2-trans-dichloroethylene, trichloroethylene (TCE), 1,1,1-trichloroethane, 1,1,2-trichloroethane (vinyl trichloride), 1,1,1-trichloroethene, 1,1,1,2-tetrachloroethane, 1,1,2,2-tetrachloroethane (acetylene tetrachloride), tetrachloroethylene (perchloroethylene; PCE), pentachloroethane, hexachloroethane, chloropropane, 1,2-dichloropropane (propylene dichloride), 1-chloro-2-propene, 1,3-cis-dichloro-1-propene, 1,3-trans-dichloropropene, trichloropropane, 1,2,3-trichloropropane, chloroprene, 2-butylene dichloride, hexachlorobutadiene, hexachlorocyclopentadiene, monochlorocyclohexane, monochlorobenzene, and combinations thereof.

3. The process of claim 1, wherein the OCM catalyst composition comprises a single metal oxide, mixtures of single metal oxides, a mixed metal oxide, mixtures of mixed metal oxides, or combinations thereof; and wherein the OCM catalyst composition is a supported OCM catalyst composition and/or a unsupported OCM catalyst composition.

4. The process of claim 3, wherein the OCM catalyst composition is a supported OCM catalyst composition comprising a support, wherein at least a portion of the OCM catalyst composition contacts, coats, is embedded in, is supported by, and/or is distributed throughout at least a portion of the support; and wherein the support comprises MgO, Al₂O₃, SiO₂, ZrO₂, TiO₂, or combinations thereof.

5. The process of claim 3, wherein the OCM catalyst composition is characterized by the general formula Aₐ₁Z_{b1}E_{c1}D_{d1}Oₓ₁; wherein A is an alkaline earth metal; wherein Z is a first rare earth element; wherein E is a second rare earth element; wherein D is a redox agent or a third rare earth element; wherein the first rare earth element, the second rare earth element, and the third rare earth element, when present, are not the same; wherein a1 is 1.0; wherein b 1 is from about 0.1 to about 10.0; wherein c 1 is from about 0 to about 10.0; and wherein d1 is from about 0 to about 10.0; and wherein x1 balances the oxidation states.

6. The process of claim 5, wherein the alkaline earth metal is selected from the group consisting of magnesium (Mg), calcium (Ca), strontium (Sr), barium (Ba), and combinations thereof; wherein the first rare earth element, the second rare earth element, and the third rare earth element can each independently be selected from the group consisting of lanthanum (La), scandium (Sc), cerium (Ce), praseodymium (Pr), neodymium (Nd), promethium (Pm), samarium (Sm), europium (Eu), gadolinium (Gd), yttrium (Y), terbium (Tb), dysprosium (Dy), holmium (Ho), erbium (Er), thulium (Tm), ytterbium (Yb), lutetium (Lu), and combinations thereof; and wherein the redox agent is selected from the group consisting of manganese (Mn), tungsten (W), bismuth (Bi), antimony (Sb), tin (Sn), cerium (Ce), praseodymium (Pr), and combinations thereof.

7. The process of claim 5, wherein the OCM catalyst composition has the general formula Srₐ₁La_{b1}E_{c1}D_{d1}Oₓ₁; wherein E is a second rare earth element other than lanthanum (La); wherein D is a third rare earth element other than lanthanum (La); wherein the second rare earth element and the third rare earth element are different; wherein a1 is 1.0; wherein b1 is from about 0.1 to about 10.0; wherein c1 is from about 0.01 to about 10.0; wherein d1 is from about 0.01 to about 10.0; and wherein x1 balances the oxidation states.

8. The process of claim 7, wherein the OCM catalyst composition has the general formula Srₐ₁La_{b1}Yb_{c1}Nd_{d1}Oₓ₁; wherein a1 is 1.0; wherein b1 is from about 0.1 to about 10.0; wherein c1 is from about 0.01 to about 10.0; wherein d1 is from about 0.01 to about 10.0; and wherein x1 balances the oxidation states.

9. The process of claim 8, wherein the OCM catalyst composition comprises a unsupported OCM catalyst composition and/or a supported OCM catalyst composition; and wherein, when the OCM catalyst composition comprises a supported OCM catalyst composition, the supported OCM catalyst composition comprises an alumina support.

10. The process of claim 5, wherein the OCM catalyst composition is a supported OCM catalyst composition comprising a MgO support; wherein the OCM catalyst composition has the general formula Srₐ₁La_{b1}Oₓ₁/MgO; wherein a1 is 1.0; wherein b1 is from about 0.1 to about 10.0; and wherein x1 balances the oxidation states.

11. The process of claim 3, wherein the OCM catalyst composition is characterized by the general formula Laₐ₂Ce_{b2}Oₓ₂; wherein a2 is 1.0; wherein b2 is from about 0.3 to about 10.0; and wherein x2 balances the oxidation states.

12. The process of claim 3, wherein the OCM catalyst composition is a supported OCM catalyst composition comprising a silica support; wherein the OCM catalyst composition is characterized by the general formula Mn—Na₂WO₄/SiO₂; wherein the supported OCM catalyst composition optionally comprises a metal oxide characterized by the general formula MOₓ₃; wherein M is a metal with redox properties; and wherein x3 balances the oxidation states.

13. The process of claim 12, wherein the metal M is selected from the group consisting of tin (Sn), antimony (Sb), bismuth (Bi), iron (Fe), chromium (Cr), molybdenum (Mo), tungsten (W), vanadium (V), tantalum (Ta), niobium (Nb), gallium (Ga), rhenium (Re), lead (Pb), cobalt (Co), nickel (Ni), copper (Cu), and combinations thereof; and wherein the supported OCM catalyst composition comprises the metal M in an amount of from about 0.1 wt. % to about 10 wt. %, based on the total weight of the supported OCM catalyst composition.

14. The process of claim 12, wherein the OCM catalyst composition has the general formula (SbOₓ₃)—Mn—Na₂WO₄/SiO₂.

15. The process of claim 1, wherein the OCM reactor is operated under autothermal conditions or isothermal conditions.

16. The process of claim 15; wherein, when the OCM reactor is operated under autothermal conditions, the OCM reactor is characterized by a feed temperature of from about 25° C. to about 400° C., thereby providing for igniting the OCM catalyst composition at an ignition temperature of from about 450° C. to about 650° C.

17. The process of claim 15; wherein, when the OCM reactor is operated under autothermal conditions, the autothermal conditions are maintained by decreasing a CH₄/O₂ molar ratio in the reactant mixture while and/or subsequent to removing an external heat supply to the OCM catalyst composition.

18. The process of claim 17; wherein, when the OCM reactor is operated under autothermal conditions, the OCM reactor is characterized by a catalyst bed temperature maintained between about 700° C. to about 1,000° C. while and/or subsequent to removing an external heat supply to the OCM catalyst composition.

19. A process for producing ethylene comprising:
(a) introducing a reactant mixture to an oxidative coupling of methane (OCM) reactor comprising an OCM catalyst composition; wherein the reactant mixture comprises methane (CH₄), oxygen (O₂), and methyl chloride (CH₃Cl); wherein CH₃Cl is present in the reactant mixture in an amount of from about 1 part per million (ppm) to about 50 ppm, based on the total volume of the reactant mixture;
(b) allowing at least a portion of the reactant mixture to contact at least a portion of the OCM catalyst composition and react via an OCM reaction to form a product mixture comprising unreacted methane and C₂₊ olefins; wherein the C₂₊ olefins comprise ethylene;
(c) recovering at least a portion of the product mixture from the OCM reactor; and
(d) recovering at least a portion of ethylene from the product mixture.

20. The process of claim 19, wherein the process for producing ethylene is characterized by improved performance when compared to an otherwise similar process that employs a reactant mixture comprising CH₄ and O₂, without the CH₃Cl; wherein the improved performance of the process is defined by the process having a C₂₊ selectivity that is increased by equal to or greater than about 2% when compared to a C₂₊ selectivity of an otherwise similar process that employs a reactant mixture comprising CH₄ and O₂, without the CH₃Cl.

21. The process of claim 19, wherein the OCM catalyst composition is characterized by the general formula Srₐ₁La_{b1}Yb_{c1}Nd_{d1}Oₓ₁; wherein a1 is 1.0; wherein b1 is from about 0.3 to about 10.0; wherein c1 is from about 0.05 to about 10.0; wherein d1 is from about 0.05 to about 10.0; and wherein x1 balances the oxidation states.

22. The process of claim 19, wherein the CH₃Cl is present in the reactant mixture in an amount of from about 20 ppm to about 50 ppm, based on the total volume of the reactant mixture.

23. The process of claim 19, wherein the OCM reactor is operated under autothermal conditions; and wherein, prior to step (b) of allowing at least a portion of the reactant mixture to contact at least a portion of the OCM catalyst composition and react via an OCM reaction, the OCM catalyst composition is pre-treated by contacting at least a portion of the OCM catalyst composition with a pre-treating mixture comprising CH₄ and from about 25 ppm to about 75 ppm CH₃Cl for a time period of from about 2 hours to about 12 hours.
